(19) United States
(12) Patent Application Publication
(10) Pub. No.: US 2010/0029674 A1
(43) Pub. Date: Feb. 4, 2010

(54) Methods of Using Phosphoantigen for the Treatment of Cancer

(75) Inventors: Jérôme Tiollier, Marseille (FR); Hélenè Scard, Marseille (FR); Cécile Bonnafous, Marseille (FR)

Correspondence Address: SALIWANCHIK LLOYD & SALIWANCHIK;A PROFESSIONAL ASSOCIATION, PO Box 142950, GAINESVILLE, FL, 32614, US

(73) Assignee: INNATE PHARMA, S.A., Marseille (FR)

(21) Appl. No.: 12/438,998

(22) PCT Filed: Nov. 16, 2007
(86) PCT No.: PCT/EP2007/062456

§ 371 Date: Feb. 26, 2009

Related U.S. Application Data
(60) Provisional application No. 60/938,020, filed on May 15, 2007.

(51) Int. Cl. A61K 31/506; C12N 5/02

(52) U.S. Cl. 514/252.18; 435/375

(57) ABSTRACT

The present invention relates to compositions and methods useful for treating a cancer in mammals, including humans. The methods and compositions typically comprise use of a chemotherapeutic agent and a γδ T cell activator such that the composition is effective for treating a cancer. Preferably the composition enhances the effect of the γδ T cell activator and/or prevents or delays the escape of a tumor from control chemotherapy, particularly an anti-angiogenic chemotherapeutic agent.

This application is a continuation-in-part of PCT/EP2006/068610, filed Nov. 17, 2006, which claims the benefit U.S. Provisional Patent Application 60/737,588, filed Nov. 17, 2005. This application also claims the benefit of U.S. Provisional Patent Application 60/938,020, filed May 15, 2007.

### FIELD OF THE INVENTION

The present invention relates to compositions and methods useful for treating a cancer in mammals, including humans. The methods and compositions typically comprise use of a chemotherapeutic agent and a γδ T cell activator, such that the composition is effective for treating a cancer. Preferably the composition enhances the effect of the γδ T cell activator and/or prevents or delays the escape of a tumor from control chemotherapy, particularly an anti-angiogenic chemotherapeutic agent.

### BACKGROUND OF THE INVENTION

Chemotherapeutic agents are widely used in the treatment of cancer, and include a wide range of biological mechanisms, including notably cytotoxic compounds and non-cytotoxics such as compounds having anti-angiogenic properties (e.g. tyrosine kinase inhibitors). Chemotherapeutic agents are in most cases the first line of treatment. Nevertheless, chemotherapeutic treatments are not effective for all patients and depending on the situation, a large percentage of patients is unresponsive or refractory. Moreover, once patients are treated with chemotherapeutic agents their tumors may “escape” and become yet more resistant to other therapies. There has therefore been an active search for drug combinations in order to improve treatment.

Angiogenesis inhibitors such as molecules targeting the VEGF/VEGF-R are in development in phase II or III trials, including BAY 43-9006 (Nexavar® (sorafenib tosylate)), leading to inhibition of tumor proliferation, was tested in phase II trial and has done encouraging results in RCC, leading to the phase III study (Ratain et al., Proc. Am. Soc. Clin. Oncol., 23:381, 2004). SU-011248 (Sunitinib, Sutent™), tested on RCC patients in whom standard therapies had failed, has shown partial responses with a progression-free of disease and was conducted in phase III trial (Motzer et al., Proc. Am. Soc. Clin. Oncol., 23:381, 2004). However, these anti-angiogenic therapies do not completely eradicate the tumor, and while they manage to control the growth of a tumor for a period of time the tumor eventually escapes control and is then resistant to the anti-angiogenic and/or other therapies. A means to prevent the escape of the tumor would be advantageous.

The therapy combinations that have generally proved effective are combinations of cytotoxic chemotherapeutic agents, for example the combination of cisplatin and vinorelbine, paclitaxel, docetaxel or gemcitabine, and the combination of carboplatin and paclitaxel in the treatment of lung cancer. One limitation of combination cytotoxic chemotherapy is that anticancer agents generally have severe side effects, even when administered individually. For example, the well known anti-cancer agent taxol causes neutropenia, neuropathy, mucositis, anemia, thrombocytopenia, bradycardia, diarrhea and nausea. Unfortunately, the toxicity of anti-cancer agents is generally additive when the drugs are administered in combination. As result, certain types of anti-cancer drugs are generally not combined. The combined toxic side-effects of those anti-cancer drugs that are administered simultaneously can place severe limitations on the quantities that can be used in combination such that it is often impossible to use enough of the combination therapy to achieve the desired synergistic effects. Therefore, there is an urgent need for agents which can be used conjointly.

The combination of immunotherapeutic agents and chemotherapeutic agents, however, has been studied only minimally. Moreover, the combination of chemotherapy and immunotherapy has generally been avoided where possible. Firstly, many cytotoxic chemotherapeutic drugs kill cells by an apoptosis, which mechanism has generally been thought to be antagonistic with immunotherapy since it is known to induce immune tolerance, and state where T cells can no longer respond to an antigen presented in the context of an immunotherapy treatment. Secondly, chemotherapeutics generally induce lymphopaenia which is thought to be detrimental to an immune response; patients previously treated with chemotherapeutics have been observed to have reduced numbers of T cells and/or greatly reduced ability to respond to a presented antigen. Moreover, it is assumed that patients treated with chemotherapeutic agents will be unable to mount an expansion of T cells when treated with an immunostimulatory compound. Tyrosine kinase inhibitors, while not necessarily directly cytotoxic to cancer cells, have also been reported to have effects on T cells, of which one example is imitanib mesylate (STI571, Glivec™, Gleevec™, Novartis, Basel, Switzerland). Imitanib mesylate is a reversible tyrosine kinase inhibitor effective in the treatment of chronic myeloid leukemia (CML), gastrointestinal stromal tumors and other tumors and binds preferentially to ATP binding sites of the c-kit protooncogene product, PDGF-R and c-Abelson kinase. Imitanib mesylate has been reported to inhibit T cell proliferation and activation, to inhibit delayed-type hypersensitivity in vivo, and to inhibit cytokine synthesis by CD4 T cells (Seggewiss et al. (2005) Blood 105 (6): 2473-2479; Dietz et al. (2004) Blood 104:1094-1099; and Gao et al. (2005) Leukemia 19:1905-1911).

The present invention provides new therapeutic modalities that can be used to enhance the effect of chemotherapies and/or prevent escape of tumors from control by chemotherapeutic and especially anti-angiogenic agents.

### SUMMARY

The present invention is based on observations during a human clinical trial using bromohydrin pyrophosphate (BrHPP, also referred to as Phosphostim), where it was observed that this compound leads to γδ T cell activation, including a strong cytokine secretion and vigorous γδ T cell expansion when was administered in chemotherapy-treated patients. Patients having been treated with one or even multiple chemotherapy regimens retained the ability to host a γδ T cell expansion.

Thus, in one aspect the invention provides a method for activating and/or inducing the proliferation of a γδ T cell in a mammal, the method comprising conjointly administering to the mammal a γδ T cell activator and a chemotherapeutic agent. In another aspect the invention encompasses a method for killing or inhibiting a proliferating cell, preferably a tumor cell in a mammal, or for enhancing the efficacy in a mammal of a chemotherapeutic agent, the method comprising conjointly administering to the mammal a γδ T cell activator and a chemotherapeutic agent. In one embodiment, the chemotherapeutic agent is effective to kill or inhibit a tumor cell. Also provided is the use of a γδ T cell activator for the manufacture of a pharmaceutical composition or medicament, wherein said pharmaceutical composition or medicament is used or administered in combination with a chemotherapeutic agent. Also encompassed are related pharmaceutical compositions and kits comprising such compositions. In one embodiment, the chemotherapeutic agent is an agent that is cytotoxic towards tumor cells. In another embodiment, the chemotherapeutic agent is a tyrosine kinase inhibitor. In another embodiment, the chemotherapeutic agent has anti-angiogenic properties but is not cytotoxic towards tumor cells. In another embodiment, the tyrosine kinase inhibitor has anti-angiogenic properties.

In another embodiment, the invention provides a method for activating and/or inducing the proliferation of a mammalian γδ T cell, the method comprising bringing a γδ T cell into contact with a γδ T cell activator, wherein the γδ T cell has previously been treated with a chemotherapeutic agent. In another embodiment, the invention provides a method for activating and/or inducing the proliferation of a γδ T cell in a mammal, or for killing or inhibiting a proliferating cell, preferably a tumor cell in a mammal, the method comprising administering to a mammal a γδ T cell activator, wherein the mammal has previously been treated with a chemotherapeutic agent. In another embodiment, the invention provides a method for activating and/or inducing the proliferation of a γδ T cell in a mammal, or for killing or inhibiting a proliferating cell, preferably a tumor cell in a mammal, the method comprising administering to the mammal a γδ T cell activator after a treatment with a chemotherapeutic agent, preferably wherein the γδ T cell activator is administered within 6 months, 3 months, 2 months or 1 month following treatment with a chemotherapeutic agent. In yet another embodiment, the γδ T cell activator is administered at least 6 months, 3 months, 2 months or 1 month following the last dose of the chemotherapeutic agent. In another embodiment, the γδ T cell activator is administered at least 6 months, 3 months, 2 months or 1 month following the beginning of the treatment with a chemotherapeutic agent, the treatment with a chemotherapeutic agent being continued. In one aspect, the chemotherapeutic agent is a compound which is cytotoxic to tumor cells. In another embodiment, the chemotherapeutic agent has anti-angiogenic properties but is not cytotoxic towards tumor cells.

In one specific aspect, the chemotherapeutic agents used in conjunction with the γδ T cell activator are of the kind that may lead to the upregulation of ligands recognized by activatory receptor on γδ T cells. While not wishing to be bound by theory, a possibility is that administration of a γδ T cell activator in combination with certain treatments such as selected chemotherapeutic agents or ionizing radiation that upregulate expression of NKG2D ligands on the surface of tumor cells can be used advantageously in treatment. Chemotherapeutic agents capable of upregulating the expression of NKG2D ligands are further described herein.

In another aspect, the chemotherapeutic agents used in conjunction with γδ T cell activator are of the tyrosine kinase inhibitor type. Such tyrosine kinase inhibitors are further described herein, including examples of tyrosine kinase inhibitors that have anti-angiogenic properties. Preferred examples oftyrosine kinase inhibitors that can be used in accordance with the invention include agents that inhibit one or more kinases selected from the group consisting of VEGFR1, VEGFR-2,3, PDGFR-beta, Flt-3, and c-Kit. In one embodiment, said tyrosine kinase inhibitors are used to treat a mammal having a tumor characterized by abnormal VEGFR1, VEGFR-2,3, PDGFR-beta, Flt-3, and c-Kit signaling. More preferably, said mammal has a tumor characterized by a mutation, preferably a gain-of-function mutation, in a VEGFR1, VEGFR-2,3, PDGFR-beta, Flt-3, and c-Kit gene or protein, for example a mutation resulting in an overactive or constitutively activated tyrosine kinase.

In one aspect the invention provides a method for activating and/or inducing the proliferation of a γδ T cell, or a method for treating a tumor, or killing or inhibiting the growth of a proliferating cell, preferably a tumor cell, in a mammal, the method comprising conjointly administering to the mammal a γδ T cell activator and an agent that inhibits one or more kinases selected from the group consisting of VEGFR1, VEGFR-2,3, PDGFR-beta, Flt-3, and c-Kit. In one aspect, the mammal has a tumor characterized by aberrant or increased tyrosine kinase signaling activity. In another aspect the mammal has a tumor characterized by a mutation in a tyrosine kinase, preferably a tyrosine kinase selected from the group consisting of VEGFR1, VEGFR-2, VEGFR-3, PDGFR-beta, Flt-3, and c-Kit. Optionally, the method comprises the additional step of determining whether a mammal has a tumor characterized by aberrant or increased tyrosine kinase signaling activity, or preferably by a mutation in a tyrosine kinase, and if said mammal has such a tumor, treating said mammal with according to the method of the invention. Optionally, the method comprises treating said mammal so as, or for so long as necessary, to bring numbers of cell expressing a mutated kinase to a predetermined level, or to undetectable levels. In one example, the γδ T cell activator is administered at least twice, wherein successive administrations of γδ T cell activator are separated by at least 7, 10, 14, 20 days, and the tyrosine kinase inhibitor is administered daily or weekly during treatment with the period of treatment with γδ T cell activator. In one example, the γδ T cell activator is administered at least twice, and on the first day of a 2-weekly, 3-weekly, 4-weekly, 5-weekly, 6 weekly, 7 weekly, 8-weekly or greater cycle, and the tyrosine kinase inhibitor is administered daily or at least one weekly during treatment with the period of treatment with γδ T cell activator.

Other exemplary tyrosine kinase inhibitors that can be used in accordance with the invention include agents that inhibit one or more kinases selected from the group consisting of abl and receptors of the same family including but not limited to bcr/abl, c-Kit and PDGFR. In one embodiment, said tyrosine kinase inhibitors are used to treat a mammal having a tumor characterized by a c-ABL, BCR-ABL, c-KIT or PDGFR gene or protein mutation, preferably a gain-of-function mutation, for example a mutation resulting in an overactive or constitutively activated tyrosine kinase, a reciprocal translocation such as the Philadelphia chromosome (bcr/abl), or an KITD816V, an imatinib-resistant activating mutation in KIT.

In one aspect the invention provides a method for activating and/or inducing the proliferation of a γδ T cell, or a method for treating a tumor, or killing or inhibiting the growth of a proliferating cell, preferably a tumor cell, in a mammal, the method comprising conjointly administering to the mammal a γδ T cell activator and an agent that inhibits one or more kinases selected from the group consisting of abl and receptors of the same family including but not limited to bcr/abl, c-Kit and PDGFR. In one aspect, the mammal has a tumor characterized by aberrant or increased tyrosine kinase signaling activity. In another aspect the mammal has a tumor characterized by a mutation in a tyrosine kinase, preferably a tyrosine kinase selected from the group consisting of abl and receptors of the same family including but not limited to bcr/abl, c-Kit and PDGFR. Optionally, the method comprises the additional step of determining whether a mammal has a tumor characterized by aberrant or increased tyrosine kinase signaling activity, or preferably by a mutation in a tyrosine kinase, and if said mammal has such a tumor, treating said mammal with according to the method of the invention. Optionally, the method comprises treating said mammal so as, or for so long as necessary, to bring numbers of cell expressing a mutated kinase to a predetermined level, or to undetectable levels. In one example, the γδ T cell activator is administered at least twice, wherein successive administrations of γδ T cell activator are separated by at least 7, 10, 14, 20 days, and the tyrosine kinase inhibitor is administered daily or weekly during treatment with the period of treatment with γδ T cell activator. In one example, the γδ T cell activator is administered at least twice, and on the first day of a 2-weekly, 3-weekly, 4-weekly, 5-weekly, 6 weekly, 7 weekly, 8-weekly or greater cycle, and the tyrosine kinase inhibitor is administered daily or at least once weekly during treatment with the period of treatment with γδ T cell activator.

The present invention also discloses particular compositions and methods that can be used to efficiently treat a tumor in a subject by a mechanism of modulation of both the host immune system and the tumor microenvironment. The invention in particular provides novel treatment regimens by which immunomodulatory compounds can be used to treat tumors in conjunction with anti-angiogenic therapies. Anti-angiogenic therapies have a cytostatic effect rather than cytotoxic and are therefore expected to control rather than eradicate tumors such that after a certain time tumors will escape control and become resistant to the anti-angiogenic therapy. γδ T cells, in contrast to anti-angiogenic therapy, have the potential to eradicate tumor cells completely in animals, whereby the tumor cells do not reappear when treatment is stopped. It is believed that this will hold true particularly when solid tumors are of limited size, as they may be when kept under control by anti-angiogenic therapies. Activation of cytotoxic lymphocytes thus provides a means to kill tumor cells during the period in which the tumor is under anti-angiogenic control, without the often additive toxicity observed with traditional cytotoxic chemotherapeutic agents. The therapeutic regimens and compositions thus provide a means to enhance the effect of immunotherapies as well as prevent the “escape” of tumor from anti-angiogenic therapy treatment. Based on experiences with γδ T cell activators, preferred regimens that provide effective immunotherapy-anti-angiogenic therapy combinations are disclosed.

The effects of many immunotherapeutic compounds are at least in part counteracted by angiogenic effects of a tumor. In particular, it is thought that angiogenesis contributes to the tumor's intrinsic resistance to infiltration by the activated effectors cells. This applies particularly to immunotherapeutic therapies that depend on extravasation of activated or proliferating immune cells, e.g. into a tumor environment.

The inventors provide that chemotherapeutic agents such as inhibitors of receptor tyrosine kinases or VEGF/VEGFR signaling pathway having effect on wide range of proliferating cells can be used in conjunction with immunomodulatory compounds that require immune cells which retain an ability to be activated and/or to proliferate.

Renal cell carcinoma represents one example of a therapeutic setting where anti-angiogenic therapies and immunotherapies can be used to effectively modulate both the host immune system and the tumor microenvironment. The most promising results in renal cell carcinoma have been obtained with molecules targeting the network of tumor vasculature by targeting angiogenesis. At the same time, γδ T cells activated by BrHPP have been show to directly lyse fresh renal cell carcinoma cells but not non-tumor cells from the same patient and in a first clinical trial have been shown to activate γδ T cell and have potential for therapeutic benefit. It has also been shown that Vδ2 T effectors are present at the renal tumor site (Viey, E., et al., Phosphostim-activated gamma delta T cells kill autologous metastatic renal cell carcinoa. J Immunol, 2005. 174(3): p. 1338-47) suggesting that these cells are able to migrate towards the inflamed and tumor tissues. The present invention therefore provides that, by using molecules such as tyrosine kinase inhibitors (raf kinase inhibitors, VEGFR1, VEGFR2, c-KIT, etc.), thalidomide or its analogue CC-5013, lenalidomide (Revlimib™, potent in vivo angiogenesis inhibitor), we could create a permissive tumor environment, overcoming the barriers created by the tumor vasculature, favoring lymphocyte extravasation and lytic function at the tumor site(s). In the long run, patients treated by anti-angiogenic agents, such as sorafenib, could develop a resistance to the biological activity of these molecules. The combination with BrHPP molecule, boosting the Vγ9Vδ2 immune population, could complete the relevant but short-term effects of novel pharmacological agents, creating a long-term anti-tumor responses. The aim of this approach would be to reduce the tumor mass acting on the blood vessels necessary to its growth and then to induce an immune response to tumor by (i) stimulation with of the Vγ9Vδ2 T lymphocytes in vivo or (ii) infusion of ex vivo autologous expanded and/or activated Vγ9Vδ2 T cells to patients.

Example of anti-angiogenic therapies include molecules targeting the VEGF/VEGF-R which are in development in phase II or III trials, and for example BAY 43-9006, all of which lead to inhibition of tumor proliferation. Bay 43-9006 was tested in phase II trial yielding encouraging results in RCC, leading to a phase III study (Escudier et al., ASCO, 23:4510, 2005). SU-011248, tested on RCC patients in whom standard therapies had failed, has shown partial responses with a progression-free of disease and is now being tested in a phase III clinical trial (Motzer et al., Proc Am Soc Clin Oncol, 23:381, 2004).

Another example of an anti-angiogenic therapy and tyrosine kinase inhibitor is imitanib mesylate (ST1571, Glivec™, Gleevec™, Novartis, Basel, Switzerland). Imitanib mesylate is a reversible tyrosine kinase inhibitor effective in the treatment of CML, gastrointestinal stromal tumors and other tumors and is a potent selective inhibitor of the tyrosine kinases ABL, ARG, PDGFR-alpha and PDGFR-beta, and c-KIT.

The inventors and/or their colleagues have previously characterized a number of immunomodulatory compounds capable of modulating the activity and proliferation of γδ T cells. These compounds (also called “phosphoantigens”) generally share a common structure in that they are organophosphate compounds. The classes having greatest potency are more particularly phosphate esters and phospho-phosphoroamidate esters. The particular compounds used by the inventors and their colleagues in a lead clinical trial comprises a pyrophosphate moiety; however other related compounds are being investigated as well, including but not limited to bisphosphonate compounds. The lead molecule of this series of compounds developed by the inventors and their colleagues is called Phosphostim™, which is at present being evaluated in two Phase I clinical trials in oncology. A number of other compounds which activate γδ T cells are known as well, although most of these act indirectly (e.g. act on other immune cells which in turn activate γδ T cells) or act directly on γδ T cells but are less potent.

In one aspect, the invention provides a method for activating and/or inducing the proliferation of a γδ T cell in a mammal, or for enhancing the extravasation of an activated γδ T cell in a mammal, the method comprising conjointly administering to the mammal a γδ T cell activator and an inhibitor of angiogenesis. In another embodiment, the invention provides a method for activating and/or inducing the proliferation of a mammalian γδ T cell, the method comprising bringing a γδ T cell into contact with a γδ T cell activator, wherein the γδ T cell has previously been treated with an inhibitor of angiogenesis. In another embodiment, the invention provides a method for activating and/or inducing the proliferation of a γδ T cell in a mammal, or for killing or inhibiting a proliferating cell, preferably a tumor cell in a mammal, the method comprising administering to a mammal a γδ T cell activator, wherein the mammal has previously been treated with an inhibitor of angiogenesis. In another aspect, the invention encompasses a method for killing or inhibiting a proliferating cell, preferably a tumor cell in a mammal, or for enhancing the efficacy in a mammal of an inhibitor of angiogenesis, the method comprising conjointly administering to the mammal a γδ T cell activator and an inhibitor of angiogenesis. Also provided is the use of a γδ T cell activator for the manufacture of a pharmaceutical composition or medicament, wherein said pharmaceutical composition or medicament is used or administered in combination with an inhibitor of angiogenesis. Also encompassed are related pharmaceutical compositions and kits comprising such compositions.

In another embodiment, the invention provides a method for activating and/or inducing the proliferation of a γδ T cell in a mammal, or for killing or inhibiting a proliferating cell, preferably a tumor cell in a mammal, the method comprising administering to the mammal a γδ T cell activator after a treatment with an inhibitor of angiogenesis, preferably wherein the γδ T cell activator is administered within 6 months, 3 months, 2 months or 1 month following treatment with an inhibitor of angiogenesis. In yet another embodiment, the γδ T cell activator is administered at least 3 months, 2 months or 1 month following treatment with an inhibitor of angiogenesis. In a preferred aspect, the anti-angiogenic agent is a tyrosine kinase inhibitor. In one example, the tyrosine kinase inhibitor is imitanib mesylate, sunitinib or sorafenib. In another aspect, the invention provides a method for enhancing the killing of a target cell comprising: (a) activating a γδ T cell by bringing said γδ T cell into contact with a γδ T cell activator; and (b) bringing the activated γδ T cell into contact with a target cell which has been contacted with a chemotherapeutic agent.

In another aspect, the invention provides a method for enhancing the killing of a target cell in a mammal comprising: (a) contacting a target cell in the mammal with a chemotherapeutic agent; and (b) activating a γδ T cell in the mammal by bringing said γδ T cell into contact with a γδ T cell activator.

In another aspect, the invention provides a method for enhancing the killing of a target cell in a mammal comprising: (a) activating a γδ T cell by bringing said γδ T cell into contact with a γδ T cell activator in vitro; (b) contacting a target cell in the mammal with a chemotherapeutic agent; and (c) administering said activated γδ T cell to the mammal.

In one aspect, the invention provides a method for activating and/or inducing the proliferation of a γδ T cell in a mammal, or for enhancing the extravasation of an activated γδ T cell in a mammal, the method comprising conjointly administering to the mammal an activated γδ T cell and an inhibitor of angiogenesis. The preparation of activated γδ T cells in culture can be carried out according to PCT patent publication no. WO 03/070921 (Innate Pharma), the disclosure of which is incorporated herein by reference.

In another aspect, the invention encompasses a method for killing or inhibiting a proliferating (e.g. tumor) cell, for enhancing the anti-tumor effect of an anti-angiogenic therapy, for enhancing the anti-tumor effect of a γδ T cell activating therapy, for preventing the escape of a tumor from control by anti-angiogenic therapy, and/or for preventing resistance of a tumor to anti-angiogenic therapy, in a mammal, the method comprising: conjointly administering to the mammal a γδ T cell activator and an inhibitor of angiogenesis. Also provided is the use of a γδ T cell activator for the manufacture of a pharmaceutical composition or medicament, wherein said pharmaceutical composition or medicament is used or administered in combination with an inhibitor of angiogenesis. Also encompassed are related pharmaceutical compositions and kits comprising such compositions.

Particularly preferred γδ T cell activating therapies that are expected to have enhanced activity with anti-angiogenic therapies are those which activate or cause the proliferation of cytotoxic γδ T cells, and particularly those that activate or cause the proliferation of γδ T cells that extravasate into a tumor environment (e.g. for the treatment of solid tumors).

Preferably the γδ T cell activator is selected from the group of: a compound capable of selectively activating a γδ T cell, a compound capable of activating a γδ T cell in a substantially pure culture of γδ T cells and a compound of Formulas I to III. In the framework of the present invention, the expression “Formulas I to III”, designate all compounds derived from Formulas I to III: I, II, IIa, III, IIIa, IIIa1, IIIa2, IIIa3, A, B, IIIb, IIIb1, IIIb2, IIIb3, C, IIc, IIIdc1, IIIc2, IIIc3, D, E, F and G. Most preferably, the compounds are selected from the list consisting of BrHPP, IPP, HDMAPP, C-HDMAPP, N-HDMAPP and H-angelyIPP.

The present invention concerns the use of a γδ T cell activator for the manufacture of a medicament, wherein the γδ T cell activator is for conjoint administration with a chemotherapeutic agent. In a particular embodiment, the chemotherapeutic agent is a tyrosine kinase inhibitor. In another particular embodiment, the chemotherapeutic agent is a tyrosine kinase inhibitor capable of inhibiting bcr/abl. In a further particular embodiment, the chemotherapeutic agent is a tyrosine kinase inhibitor that is a competitive inhibitor at the ATP-binding site of Bcr/Abl. Optionally, the chemotherapeutic agent is a tyrosine kinase inhibitor selected from the group consisting of imatinib, SU4312, XL647, XL1999, PKC412, AEE788, OSI-930, OSI-817, DMPQ, MLN518, lestaurinib gefitinib, OSI-774 lapatinib. PD-166326, NSC 680410, tyrphostin AG 957, AP-23464, AP-234604, SKI-606, dasatinib, nilotinib, NS-187, and CGP16030. In a preferred embodiment, the chemotherapeutic agent is a tyrosine kinase inhibitor selected from the group consisting of imatinib, dasatinib, and nilotinib. Optionally, the tyrosine kinase inhibitor does not impair the proliferation of γδ T cells. In particular, treatment with said tyrosine kinase inhibitor together with a γδ T cell activator maintains the residual disease below the detection limit as established using PCR gene amplification technique. Preferably, said medicament is for the treatment of a tumor. More preferably, said medicament is for the treatment of CML, ALL or GIST. In particular, said medicament is for the treatment of a mammal determined to have a tumor characterized by aberrant or increased kinase activity signaling activity. In particular, said medicament is has a toxicity not higher than the toxicity of the tyrosine kinase inhibitor alone.

In a particular embodiment, the chemotherapeutic agent inhibits a receptor tyrosine kinase selected from the group consisting of VEGFR1, VEGFR-2,3 PDGFR-alpha, PDGFR-beta, CSF-1,RET, Flt-3, c-Kit, p38 alpha and FGFR-1. Optionally, the γδ T cell activator is administered at least twice, wherein successive administrations of γδ T cell activator are separated by at least 7, 10, 14 or 20 days, and the tyrosine kinase inhibitor is administered daily or weekly during treatment with the period of treatment of the γδ T cell activator. Preferably, the γδ T cell activator is administered within 3 months after a treatment with the chemotherapeutic agent. In particular, said γδ T cell activator and said chemotherapeutic agent are administered in an amount effective to induce proliferation of γδ T cells in said mammal. Preferably, said γδ T cell activator and said chemotherapeutic agent are administered in an amount effective to induce activation of γδ T cells in said mammal. Optionally, the mammal is a human. Optionally, the mammal has a tumor. Preferably, the tumor is characterized by aberrant or increased tyrosine kinase signaling activity. The tumor can also characterized by a mutation in a tyrosine kinase. Preferably, the mammal is treated for so long as necessary to bring numbers of cell expressing a mutated kinase to a predetermined level, or to undetectable levels. Optionally, at least two treatments of γδ T cell activator are administered to said mammal. Optionally, the chemotherapeutic agent is selected from the group consisting of the alkylating agents of the metal salts type. The chemotherapeutic agent can be oxaliplatin. Optionally, the chemotherapeutic agent is a topoisomerase inhibitor. The chemotherapeutic agent can be irinotecan. Optionally, the chemotherapeutic agent is selected from the group consisting of folate analogs, pyrimidine analogues and purine analogues. The chemotherapeutic agent can be fluorouracil (5-FU). In a particular embodiment, the mammal suffers from colon cancer.

The present invention also concerns a pharmaceutical composition comprising a γδ T cell activator compound and imatinib.

In addition, the present invention concerns a pharmaceutical composition comprising a tyrosine kinase inhibitor and a γδ T cell activator is selected from the group consisting of compounds of Formula IIIc. Preferably, said tyrosine kinase inhibitor is selected from the group consisting of imatinib, SU4312, XL647, XL999, PKC412, AEE788, OSI-930, OSI-817, DMPQ, MLN518, lestaurinib geefitinit) OSI-774, lapitiinib. PD-166326, NSC 680410, tyrphostin AG 957, AP-23464, AP-234604, SKI-606, dasatinib, nilotinib, NS-187, and CGP16030.

The present invention further concerns a pharmaceutical composition comprising a γδ T cell activator compound and a tyrosine kinase inhibitor, wherein the toxicity of the pharmaceutical composition is not higher than the toxicity of the tyrosine kinase inhibitor alone. Preferably, the tyrosine kinase inhibitor is capable of inhibiting a receptor tyrosine kinase selected in the group consisting of VEGFR-1, VEGFR-2, VEGFR-3, PDGFR-alpha, PDGFR-beta, CSF-1R, Flt-3, c-Kit and RET.

The present invention concerns a composition comprising a γδ T cell activator compound and a chemotherapeutic agent for the treatment of a tumor. In particular, the tumor can be a tumor characterized by a gene or protein mutation selected from the group consisting of c-ABL, BCR-ABL, c-KIT and PDGFR and the chemotherapeutic agent is a tyrosine kinase inhibitor. In a particular embodiment, said tyrosine kinase inhibitor is capable of inhibiting a receptor tyrosine kinase selected from the group consisting of VEGFR-1, VEGFR-2, VEGFR-3, PDGFR-alpha, PDGFR-beta, Flt-3, c-Kit, p38 alpha, RET, c-RAF, b-RAF, bcr/abl and FGFR-1. In another particular embodiment, said tyrosine kinase inhibitor can also be capable of inhibiting a receptor tyrosine kinase selected from the group consisting of abl, bcr/abl, c-Kit and PDGFR. In an additional particular embodiment, said tyrosine kinase inhibitor is selected from the group consisting of imatinib, SU4312, XL647, XL-999, PKC412, AEE788, OSI-930, OSI-817, DMPQ, MLN518, lestaurinib, gefitinib, OSI-774, lapatinib, PD-166326, NSC 680410, tyrphostin AG 957, AP-23464, AP-234604, SKI-606, dasatinib, nilotinib, NS-187, and CGP16030. Preferably, the chemotherapeutic agent is imatinib.

The present invention concerns the use of a γδ T cell activator for the manufacture of a medicament for the treatment of a tumor, wherein the γδ T cell activator is for conjoint administration with a chemotherapeutic agent. In particular, the tumor can be a tumor characterized by a gene or protein mutation selected from the group consisting of c-ABL, BCR-ABL, c-KIT or PDGFR. Preferably, the chemotherapeutic agent is a tyrosine kinase inhibitor. In a particular embodiment, said tyrosine kinase inhibitor is capable of inhibiting a receptor tyrosine kinase selected from the group consisting of VEGFR-1, VEGFR-2, VEGFR-3, PDGFR-alpha, PDGFR-beta, Flt-3, c-Kit, p38 alpha, RET, c-RAF, b-RAF, bcr/abl and FGFR-1. In another particular embodiment, said tyrosine kinase inhibitor is capable of inhibiting a receptor tyrosine kinase selected from the group consisting of abl, bcr/abl, c-Kit and PDGFR. In an additional particular embodiment, said tyrosine kinase inhibitor is a competitive inhibitor at the ATP-binding site of Bcr/Abl. Optionally, said tyrosine kinase inhibitor is selected from the group consisting of imatinib, PD-166326, NSC 680410, tyrphostin, AP-23464, AP-234604, SKI-606, dasatinib, nilotinib, NS-187, and CGP16030. Preferably, the chemotherapeutic agent is imatinib. Optionally, the γδ T cell activator is administered within 3 months after treatment with the chemotherapeutic agent. Said γδ T cell activator and said chemotherapeutic agent can be administered in an amount effective to induce proliferation of γδ T cells in said mammal. Optionally, the chemotherapeutic agent is selected from the group consisting of the alkylating agents of the metal salts type, preferably oxaliplatin. Optionally, the chemotherapeutic agent is a topoisomerase inhibitor, preferably irinotecan. In a particular embodiment, the tumor is a colon cancer.

The present invention concerns a method of treating a subject having a proliferative disease comprising administering to the subject a tyrosine kinase inhibitor and a γδ T cell activator, wherein the tyrosine kinase inhibitor does not impair the proliferation of γδ T cells. In a preferred embodiment, the tyrosine kinase inhibitor is capable of inhibiting a receptor tyrosine kinase selected from the group consisting of VEGFR-1, VEGFR-2, VEGFR-3, PDGFR-beta, Flt-3 and c-Kit. More preferably, the tyrosine kinase inhibitor is sorafenib. In another preferred embodiment, the tyrosine kinase inhibitor is capable of inhibiting a receptor tyrosine kinase selected from the group consisting of VEGFR-1, VEGFR-2, VEGFR-3, PDGFR-alpha, PDGFR-beta, CSF-1R, Flt-3, RET and c-Kit and a γδ T cell activator, wherein the tyrosine kinase inhibitor does not impair the proliferation of γδ T cells. Preferably, the tyrosine kinase inhibitor is sunitinib. In an additional preferred embodiment, the tyrosine kinase inhibitor is capable of inhibiting a receptor tyrosine kinase selected from the group consisting of VEGFR-1, PDGFR, c-Kit and bcr/abl and a γδ T cell activator, wherein the tyrosine kinase inhibitor does not impair the proliferation of γδ T cells. More preferably, the tyrosine kinase inhibitor is imatinib.

The present invention concerns a method of treatment of a subject having a proliferative disease, comprising administering to the subject a tyrosine kinase inhibitor and a γδ T cell activator, wherein treatment with an antiangiogenic agent or a tyrosine kinase inhibitor together with a γδ T cell activator maintains the residual disease below the detection limit as established using PCR gene amplification technique. Preferably, the tyrosine kinase inhibitor is capable of inhibiting a receptor tyrosine kinase selected from the group consisting of bcr/abl, c-Kit and PDGFR.

The present invention concerns a method of treating a subject having CML, ALL or GIST, comprising administering to the subject a tyrosine kinase inhibitor capable of inhibiting a receptor tyrosine kinase selected from the group consisting of bcr/abl receptors of the same family such as c-Kit and PDGFR in combination with a γδ T cell activator. Preferably, said tyrosine kinase inhibitor is imatinib.

The present invention concerns a method of treating a subject having mRCC, RCC or GIST, comprising administering to the subject sunitinib in combination with a γδ T cell activator. The present invention also concerns a method of treating a subject having RCC or mRCC comprising administering to the subject sorafenib in combination with a γδ T cell activator.

The present invention concerns the use of a γδ T cell activator and a tyrosine kinase inhibitor capable of inhibiting bcr/abl for the manufacture of a medicament preferably for the treatment of a patient having a tumor characterized by a gene or protein mutation selected from the group consisting of c-ABL, BCR-ABL, c-KIT and PDGFR.

The present invention also concerns the use of a γδ T cell activator and a tyrosine kinase inhibitor for the treatment of a lymphoma or leukemia, in particular CML. Preferably, the tyrosine kinase inhibitor is selected from the group consisting of imatinib, PD-166326, NSC 680410, tyrphostin, AP-23464, AP-234604, SKI-606, dasatinib, nilotinib, NS-187, and CGP16030.

The present invention concerns a method for treating a cancer comprising determining whether a mammal has a tumor characterized by aberrant or increased kinase activity signaling activity, or preferably by a mutation in a tyrosine kinase, and if said mammal has such a tumor, treating said mammal with according to the method of the invention.

The present invention also concerns a method for treating a cancer comprising administering to a subject in need thereof a γδ T cell activator and sunitinib wherein the γδ T cell activator is administered at least twice, the successive administrations of γδ T cell activator being separated by at least 7, 10, 14, 20 days and sunitinib is administered daily or weekly during treatment with the period of treatment with γδ T cell activator.

In addition, the present invention concerns a method for enhancing the killing of a target cell comprising:

activating a γδ T cell by bringing said γδ T cell into contact with a chemotherapeutic agent and

activating a γδ T cell in the mammal by bringing said γδ T cell into contact with a γδ T cell activator.

The present invention concerns a method for enhancing the killing or a target cell in a mammal comprising:

activating a γδ T cell in the mammal by bringing said γδ T cell into contact with a γδ T cell activator in vitro;

contacting a target cell in the mammal with a chemotherapeutic agent; and

administering said activated γδ T cell to the mammal.

The present invention concerns the use of a γδ T cell activator for the manufacture of a medicament for inducing the proliferation of γδ T cells in a mammal, wherein the γδ T cell activator is for conjoint administration with an inhibitor of angiogenesis. In particular, the γδ T cell activator can be administered within 1 month after a treatment with an inhibitor of angiogenesis, and the inhibitor of angiogenesis can be a tyrosine kinase inhibitor Optionally, said γδ T cell activator and said inhibitor of angiogenesis are administered in an amount effective to induce proliferation of γδ T cells in said mammal. Optionally, said γδ T cell activator and said inhibitor of angiogenesis agent are administered in an amount effective to induce activation of γδ T cells in said mammal. Optionally, the inhibitor of angiogenesis inhibits a receptor tyrosine kinase selected from the group consisting of VEGFR1, VEGFR-2, VEGFR-3, PDGFR-beta, Flt-3, c-Kit, p38 alpha and FGFR-1. The inhibitor of angiogenesis can be selected from the group consisting of integrin inhibitors, metalloproteinases (MPP), farnesylation inhibitors. The inhibitor of angiogenesis can be selected from the group consisting of cilengitide, marinastat, metastat, lonafarnib and tipifarnib. The inhibitor of angiogenesis can be sorafenib tosylate. Optionally, the mammal suffers from a renal cell tumor. The inhibitor of angiogenesis can be imitanib mesylate. Optionally, the mammal suffers from CML.

In an embodiment of the use of the invention, said γδ T cell activator is administered systemically. Said γδ T cell activator can activate or stimulate a Vγ9Vδ2 T cell. Said γδ T cell activator can be a selective γδ T cell activator. Said γδ T cell activator can be a compound selected from the group consisting of the compounds of Formula I to III. Said γδ T cell activator can also be a compound selected from the group consisting of the compounds of Formula IIIa to IIIc. In particular, said γδ T cell activator can be a compound selected from the group consisting of BrHPP (A) and EpoxPP (C). Alternatively, said γδ T cell activator can be a compound selected from the group consisting of HDMAPP (D), C-HDMAPP (E), N-HDMAPP (F) and C-angelyl (G).

The present invention concerns the use of a γδ T cell activator for the manufacture of a medicament for the treatment of a tumor, wherein the γδ T cell activator is for conjoint administration with an inhibitor of angiogenesis. Preferably, the γδ T cell activator is administered within 1 month after treatment with a chemotherapeutic agent, and wherein the antiangiogenic agent is a tyrosine kinase inhibitor. In particular, said γδ T cell activator and said inhibitor of angiogenesis are administered in an amount effective to induce proliferation of γδ T cells in said mammal. Optionally, the inhibitor of angiogenesis inhibits a receptor tyrosine kinase selected from the group consisting of VEGFR1, VEGFR-2, VEGFR-3, PDGFR-beta, Flt-3, c-Kit, p38 alpha and FGFR-1. Preferably, the inhibitor of angiogenesis is sorafenib tosylate. Optionally, the tumor is a renal cell tumor. Alternatively, the inhibitor of angiogenesis can be imitanib mesylate. Optionally, the tumor is CML.

For the use of the present invention, said γδ T cell activator is preferably a selective γδ T cell activator. Said γδ T cell activator can be a compound selected from the group consisting of the compounds of Formula I to III. Said γδ T cell activator is preferably a compound selected from the group consisting of the compounds of Formula IIIa to IIIc. Optionally, said γδ T cell activator is a compound selected from the group consisting of BrHPP (A) and EpoxPP (C). Alternatively, said γδ T cell activator is a compound selected from the group consisting of HDMAPP (D), C-HDMAPP (E), N-HDMAPP (F) and C-angelyl (G).

The present invention concerns a kit comprising a pharmaceutical composition comprising a γδ T cell activator and a chemotherapeutic agent, said compositions at effective doses to treat a tumor when used together in combination therapy. Preferably, said chemotherapeutic agent is an agent according to the present invention. Said chemotherapeutic agent and said γδ T cell activator can be administered simultaneously. Alternatively, said chemotherapeutic agent and said γδ T cell activator are administered separately.

Finally, the present invention concerns method of treating a subject comprising administering to the subject a tyrosine kinase inhibitor and a γδ T cell activator, wherein the tyrosine kinase inhibitor is administered in an effective amount such that the tyrosine kinase inhibitor does not significantly impair the patient's γδ T cell proliferative response to treatment with the γδ T cell activator.

### DESCRIPTION OF THE FIGURES

FIG. 1: Dose range activation of γδ T cells in non-human primates has been demonstrated following iv injection of BrHPP during GLP pharmacology studies in non-human primates despite variability of the initial response in individual monkeys (see FIG. 1). The effect of phosphostim (in mg/kg) plus IL-2 is detected starting from 2.4 mg/kg, a plateau seems to be reached at 97 mg/kg (EC50 in vivo about 60 mg/kg). γ9δ2 T cell levels in blood reach a peak between day 5 and 9 after injection and return to basal level at day 14 in most animals (except in some animals treated with a high dose of BrHPP) with no evidence of accumulation of γ9δ2 cells in lymphoid organs.

FIG. 2: γδ T cell proliferation (Tγδ amplification) in non-human primates treated with BrHPP alone or in combination with Gleevec, based on one group of 6 animals treated with BrHPP in combination with imatinib mesylate, and a control group (6 animals) with BrHPP alone as a reference. Animals treated with the combination are represented by white bars whereas anials treated with BrHPP alone are represented by the black bars. γδ T cell proliferation is not significantly different when BrHPP is administered alone or in combination with Gleevec, confirming that Gleevec does not significantly impair the proliferation of phosphoantigen stimulated γδ T cells.

FIG. 3: Assessment of the capacity of several tyrosine kinase inhibitors (Gleevec, Sorafenib and Sutent) to modify the amplificative properties of BrHPP on human Vγ2/Vγ9 T cells (Tγδ amplification) in NOD SCID mice. 4 groups of 5 mice each received BrHPP (50 mg/kg, i.p.) on day 0 and IL-2 (2M/m², s.c.) every day from day 0 to day 4. The treated group was treated with a TKI from day 0 for 3 to 5 days. Vδ2/Vγ9 T cell proliferation indicated that the injection of a TKI at the beginning of the treatment with BrHPP does not impair γδ T cell proliferation.

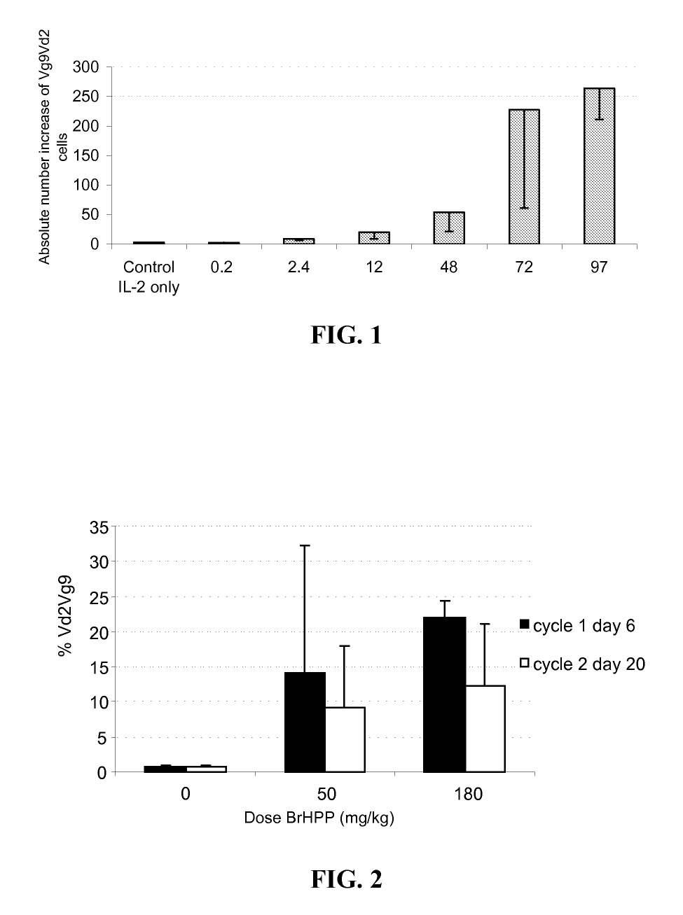

### DETAILED DESCRIPTION

### Definitions

Where “comprising” is used, this can preferably be replaced by “consisting essentially of”, more preferably by “consisting of”.

As used in the specification, “a” or “an” may mean one or more. As used in the claim(s), when used in conjunction with the word “comprising”, the words “a” or “an” may mean one or more than one. As used herein “another” may mean at least a second or more.

Where hereinbefore and hereinafter numerical terms are used, they are meant to include the numbers representing the upper and lower limits. For example, “between 1 and 3” stands for a range “from and including 1 up to and including 3”, and “in the range from 1 to 3” would stand for “from and including 1 up to and including 3”. The same is true where instead of numbers (e.g. 3) words denoting numbers are used (e.g. “three”).

“Weekly” stands for “about once a week” (meaning that more than one treatment is made with an interval of about one week between treatments), the about here preferably meaning ±1 day (that is, translating into “every 6 to 8 days”); most preferably, “weekly” stands for “once every 7 days”.

“3-weekly” or “three-weekly” stands for “about once every three weeks” (meaning that more than one treatment is made with an interval of about three weeks between treatments), the about here preferably meaning ±3 days (that is, translating into every 18 to 24 days); most preferably, “weekly” stands for “once every 21 days” (=every third week).

The term “about” or “approximately” usually means within 20%, more preferably within 10%, and most preferably still within 5% of a given value or range. Alternatively, especially in biological systems (e.g., when measuring an immune response), the term “about” means within about a log (i.e., an order of magnitude) preferably within a factor of two of a given value.

Within the context of the present invention, the expressions “stimulating the activity of γδ T cells”, “activating γδ T cells” and “regulating the activity of γδ T cells” designate causing or favoring an increase in the number and/or biological activity of such cells in a subject. Stimulating and regulating thus each include without limitation modulating (e.g., stimulating) expansion of such cells in a subject and/or, for instance, triggering of cytokine secretion (e.g., TNFα or IFNγ). γδ T cells normally represent between about 1-10% of total circulating lymphocytes in a healthy adult human subject. The present invention can be used to significantly increase the γδ T cells population in a subject, particularly to reach at least 30% of total circulating lymphocytes, typically 40%, more preferably at least 50% or 60%, or from 50% to 90%. Regulating also includes, in addition or in the alternative, modulating the biological activity of γδ T cells in a subject, particularly their cytolytic activity or their cytokine-secretion activity. The invention defines novel conditions and strategies for increasing the biological activity of γδ T cells towards target cells.

In accordance with the present invention there may be employed conventional molecular biology, microbiology, and recombinant DNA techniques within the skill of the art. Such techniques are well-known and are explained fully in the literature. See, e.g., Sambrook, Fritsch and Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and TI (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

As used herein, the terms “conjoint”, “in combination” or “combination therapy”, used interchangeably, refer to the situation where two or more therapeutic agents affect the treatment or prevention of the same disease. The use of the terms “conjoint”, “in combination” or “combination therapy” do not restrict the order in which therapies (e.g., prophylactic or therapeutic agents) are administered to a subject with the disease. A first therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject with a disease.

When one or more agents are used in combination in a therapeutic regimen, there is no requirement for the combined results to be additive of the effects observed when each treatment is conducted separately. Although at least additive effects are generally desirable, any increased effect, for example an anti-cancer effect, above one of the single therapies would be of benefit. Also, there is no particular requirement for the combined treatment to exhibit synergistic effects, although this is certainly possible and advantageous.

In one aspect, the inventors provide that a γδ T cell activator can be administered to a human in conjunction with an anti-angiogenic agent in order to induce activation and/or proliferation of γδ T cells, and disclose that said therapies can advantageously be used in combination to enhance the effects of the respective therapies and moreover to prevent a tumor from becoming resistant to anti-angiogenic therapy. Activation and/or expansion of γδ T cells in vivo is expected to be particularly useful in the treatment of a tumor, preferably a solid tumor, wherein a γδ T cell activator is administered to a warm-blooded animal, especially a human, preferably a human in need of such treatment, in a therapeutically effective amount. Where cytokines are used additionally (e.g. conjointly with the γδ T cell activator) for optimal γδ T cell proliferation, they may be but need not be administered on the same day as the γδ T cell activator, but are preferably administered within a few days of the γδ T cell activator (e.g. during the first 5 days, or during the first 10 days following administration of the γδ T cell activator when the activator is a direct γδ T cell activator).

A variety of cancers and other proliferative diseases including, but not limited to, the following can be treated using the methods and compositions of the invention:

carcinoma, including that of the bladder, breast, colon (e.g. colorectal cancer), kidney (e.g. renal cell cancer, mRCC), liver, lung (e.g. non-small cell lung cancer), ovary, pancreas, stomach, cervix, thyroid, urethra, fallopian tube, pelvis, prostate, testicules, peritoneal cavity, oesophage, gastrointestinal apparatus (e.g. GIST) and skin, including squamous cell carcinoma;

tumors of mesenchymal origin, including fibrosarcoma and rhabdomyoscarcoma;

other tumors, including melanoma, seminoma, teratocarcinoma, neuroblastoma and glioma, recurrent glioblastoma multiforme (GBM);

tumors of the central and peripheral nervous system, including astrocytoma, neuroblastoma, glioma, and schwannomas, Brain and Central Nervous System Tumors, Head and Neck Cancer, Cervical Cancer, Malignant Peripheral Nerve Sheath Tumors;

tumors of mesenchymal origin, including fibrosarcoma, rhabdomyoscaroma, and osteosarcoma;

other tumors, including melanoma, xeroderma pigmentosum, keratoacarcinoma, seminoma, thyroid follicular cancer and teratocarcinoma;

leukemias such as, but not limited to, acute leukemia, acute lymphocytic leukemia (ALL), acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukaemia (CML), chronic lymphocytic leukemia, hairy cell leukemia; polycythemia vera, chronic eosinophilic leukemia (CEL), Hypereosinophilic Syndrome;

lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma.

Most preferably, the cancers or other proliferative disease to be treated is selected from the group consisting of: CML (chronic myelocytic leukaemia), gastrointestinal stromal tumors (GIST), acute lymphocytic leukaemia (ALL), and CMML (Chronic Myelomonocytic Leukemia), renal cell carcinoma (RCC), and metastatic renal cell carcinoma (mRCC).

Where hereinbefore and subsequently a tumor, a tumor disease, a carcinoma or a cancer are mentioned, also metastasis in the original organ or tissue and/or in any other location are implied alternatively or in addition, whatever the location of the tumor and/or metastasis is.

The present invention provides improved means of preventing the escape of a tumor, particularly a solid tumor. The method of the invention therefore also provides methods of prolonging or enhanced survival in a human patient with a tumor. The method also provides a means for preventing the progression of a tumor treated with a chemotherapeutic agent. In another embodiment the invention provides a method of preventing a tumor or a tumor cell from becoming resistant to treatment with a chemotherapeutic agent.

In a preferred aspect, the γδ T cell activator may increase the biological activity of a γδ T cell, preferably increasing the activation of a γδ T cell, particularly increasing cytokine secretion from a γδ T cell or increasing the cytolytic activity of a cytotoxic γδ T cell, and/or stimulating the proliferation of a γδ T cell. Preferred γδ T cell activators include a composition comprising a compound of the formula I, especially a γδ T cell activator according to formulas I to III. In the framework of the present invention, the expression “Formulas I to III”, designate all compounds derived from Formulas I to III: I, II, IIa, III, IIa, IIIa1, IIIa2, IIIa3, A, B, IIIb, IIIb1, IIIb2, IIIb3, C, IIc, IIIc1, IIIc3, D, E, F and G. Most preferably, the γδ T cell activator is selected from the list consisting of BrHPP, IPP, HDMAPP, C-HDMAPP, N-HDMAPP and H-angelyIPP. The activator is administered in an amount and under conditions sufficient to increase the activity γδ T cells in a subject, preferably in an amount and under conditions sufficient to increase cytokine secretion by γδ T cells and/or to increase the cytolytic activity of γδ T cells. In typical embodiments, a γδ T cell activator allows the cytokine secretion by γδ T cells to be increased at least 2, 3, 4, 10, 50, 100-fold, as determined in vitro.

Method for detecting activation of a γδ T cell will be carried out according to standard methods. For example, cytokine secretion and cytolytic activity can be assessed using any appropriate in vitro assay, or those provided in the examples herein. For example, cytokine secretion can be determined according to the methods described in Espinosa et al. (J. Biol. Chem., 2001, Vol. 276, Issue 21, 18337-18344), describing measurement of TNF-α release in a bioassay using TNF-α sensitive cells. Briefly, 10⁴ γδ T cells/well were incubated with stimulus plus 25 units of IL2/well in 100 μl of culture medium during 24 h at 37° C. Then, 50 μl of supernatant were added to 50 μl of WEHI cells plated at 3×10⁴ cells/well in culture medium plus actinomycin D (2 μg/ml) and LiCl (40 mM) and incubated for 20 h at 37° C. Viability of the TNF-α-sensitive cells and measured with a 3-(4,5-dimethylthiazol-2-yl)-2,5-diphenyltetrazolium bromide assay. 50 μl of 3-(4,5-dimethylthiazol-2-yl)-2,5-diphenyktetrazoliumbromide (Sigma; 2.5 mg/ml in phosphate-buffered saline) per well were added, and after 4 h of incubation at 37° C., 50 μl of solubilization buffer (20% SDS, 66% dimethyl formamide, pH 4.7) were added, and absorbance (570 nm) was measured. Levels of TNF-α release were then calculated from a standard curve obtained using purified human rTNF-α (PeproTech, Inc., Rocky Hill, N.J.). Interferon-γ released by activated T cells was measured by a sandwich enzyme-linked immunosorbent assay. 5×10⁴ γδ T cells/well were incubated with stimulus plus 25 units of IL2/well in 100 μl of culture medium during 24 h at 37° C. Then, 50 μl of supernatant were harvested for enzyme-linked immunosorbent assay using mouse monoclonal antibodies (BIOSOURCE, Camarillo, Calif.).

A preferred assay for cytolytic activity is a ⁵¹Cr release assay. In exemplary assays, the cytolytic activity of γδ T cells is measured against autologous normal and tumor target cell lines, or control sensitive target cell lines such as Daudi and control resistant target cell line such as Raji in 4 h ⁵¹Cr release assay. In a specific example, target cells were used in amounts of 2×10³ cells/well and labeled with 100 μCi ⁵¹Cr for 60 minutes. Effector/Target (E/T) ratio ranged from 30:1 to 3.75:1. Specific lysis (expressed as percentage) is calculated using the standard formula

[(experimental-spontaneous release/total-spontaneous release)×100].

Dosage of the γδ T cell activator can be a single administration or in multiple administrations. If multiple administrations are provided, the administrations are generally separated by a period of time sufficient to prevent “exhaustion” of the γδ T cells. Exhaustion can be characterized by reduction in ability to produce cytokines or to proliferate in response to the γδ T cell activator, in comparison to that observed when the γδ T cells are treated with a first dose of the activator.

In preferred aspects, one first dose of a γδ T cell activator is administered, and one or more (preferably at least two) further doses of γδ T cell activator are administered, preferably more than one further treatments, especially with an interval between the treatments of more than 2, 4, 6, 8, 12, 24 or 36 weeks after the preceding treatment with theγδ T cell activator, or preferably after about three to about 24 weeks, most preferably about two to about eight weeks after the preceding treatment, respectively.

### Administration of γδ T Cell Activators of Formula I to III

Preferably, dosage (single administration) of a compound of formula I for treatment is between about 1 μg/kg and about 1.2 g/kg.

It will be appreciated that the above dosages related to a group of compounds, and that each particular compound may vary in optimal doses, as further described herein for exemplary compounds. Nevertheless, compounds are preferably administered in a dose sufficient to significantly increase the biological activity of γδ T cells or to significantly increase the γδ T cell population in a subject. Said dose is preferably administered to the human by intravenous (i.v.) administration during 2 to 180 min, preferably 2 to 120 min, more preferably during about 5 to about 60 min, or most preferably during about 30 min or during about 60 min.

In preferred exemplary compounds, a compound of formula II to III is administered in a dosage (single administration) between about 0.1 mg/kg and about 1.2 g/kg, preferably between about 10 mg/kg and about 1.2 g/kg, more preferably between about 5 mg/kg and about 100 mg/kg, even more preferably between about 5 μg/kg and 60 mg/kg. Most preferably, dosage (single administration) for three-weekly or four-weekly treatment (treatment every three weeks or every third week) is between about 0.1 mg/kg and about 1.2 g/kg, preferably between about 10 mg/kg and about 1.2 g/kg, more preferably between about 5 mg/kg and about 100 mg/kg, even more preferably between about 5 μg/kg and 60 mg/kg. In the framework of the present invention, the expression “Formula II to III”, designate all compounds derived from Formulas II to III: II, IIa, III, IIIa, IIIa1, IIIa2, IIIa3, A, B, IIIb, IIIb1, IIIb2, IIIb3, C, IIc, IIIc1, IIIc2, IIIc3, D, E, F and G. This dose is preferably administered to the human by intravenous (i.v.) administration during 2 to 180 min, preferably 2 to 120 min, more preferably during about 5 to about 60 min, or most preferably during about 30 min or during about 60 min.

In preferred exemplary compounds, a compound of formula IIIa, IIIb or IIIc, is administered in a dosage (single administration) between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg. Most preferably, dosage (single administration) for three-weekly or four-weekly treatment (treatment every three weeks or every third week) is between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg. This dose is preferably administered to the human by intravenous (i.v.) administration during 2 to 180 min, preferably 2 to 120 min, more preferably during about 5 to about 60 min, or most preferably during about 30 min or during about 60 min.

### Methods of Administration of Conjoint Therapy

As further detailed herein, the present invention makes it possible that the chemotherapeutic agent and said γδ T cell activator are both administered to said mammal within a relatively short period of time. The chemotherapeutic agent and said γδ T cell activator can be administered within 8 weeks, 6 weeks, 4 weeks, 3 weeks, 2 weeks, 1 week, 48 hours, 24 hours, or 6 hours of each other, or even simultaneously. In one example, the chemotherapeutic agent is administered prior to administration of the γδ T cell activator. Most preferably the γδ T cell activator is administered between about 2 weeks and about 8 weeks after administration of the chemotherapeutic agent. In certain preferred embodiments, the chemotherapeutic agent is administered at least weekly (e.g. once a week or more than once a week, for example daily), and the γδ T cell activator is administered within 1, 2, 3, 4, 5, 6 or 7 days of administration of the chemotherapeutic agent. Most preferably the chemotherapeutic agent is administered daily or at least twice per week and the γδ T cell activator is administered simultaneously with the chemotherapeutic agent, or within 6 hours, 12 hours, 24 hours or 48 hours of administration of the chemotherapeutic agent. As mentioned successive administrations of the γδ T cell activator are separated by a period of time sufficient for the γδ T cells to be activated again.

In certain preferred embodiments, the therapeutic regimen is such that an individual is treated with a γδ T cell activator during continued dosing of the chemotherapeutic agent. Such conjoint administration regimens can be used to prevent a tumor from becoming resistant to the chemotherapeutic agent. For example the therapeutic regimen can be designed so that an individual is treated with a course of chemotherapy (or radiotherapy) in one or in a plurality of doses, followed by administration of a γδ T cell activator (with or without conjoint administration of a cytokine), followed by a second course of chemotherapy in one or in a plurality of doses. The first and second course of chemotherapy may involve the same or a different treatment or agent. The γδ T cell activator is preferably administered shortly after (preferably between about 2 and 8 weeks after) the preceding dose of chemotherapy, and preferably at least about 2, 3, 4, 6 or 8 weeks before the next dose of the second chemotherapy course.

While it is possible to administer a pharmaceutical composition comprising said chemotherapeutic agent and said γδ T cell activator to an individual together, said a chemotherapeutic agent and said γδ T cell activator will generally be administered by separately and are administered by the same or different routes of administration. For example the chemotherapeutic agent is administered orally and the γδ T cell activator is administered systemically, preferably by intravenous (iv) route, or where both the chemotherapeutic agent and the γδ T cell activator are administered systemically, preferably by intravenous (iv) route.

### Treatment Cycles

Treatment cycles (where the γδ T cell activator is administered in more than one dose) can be carried out in a number of ways. The γδ T cell activator can be administered only once to the individual. In another aspect, the γδ T cell activator is administered in multiple doses, the administration of successive doses of the γδ T cell activator is separated by at least 2, 3 or 4 or more weeks. Generally, the γδ T cell rate (number of γδ T cells), is allowed to return to substantially basal rate prior to a second administration of the compound. At least about one week, but more preferably at least about two weeks, or up to eight weeks are required for a patient's γδ T cell rate to return to substantially basal rate.

When the γδ T cell activator is used with conjoint chemotherapeutic treatment, the precise regimen may vary depending on the particular chemotherapy or anti-angiogenic therapy. Generally, however, administration of the γδ T cell activator must be devised so as to be compatible with the regimen, particularly with respect to minimum number of weeks required between successive administrations of γδ T cell activator (e.g. generally at least 2, 3 or 4 or more weeks). For example, the γδ T cell activator can be administered only once to the individual, or preferably only once within (e.g. during or after) a particular course of chemotherapy treatment (a course of chemotherapy generally involving a plurality of dosage cycles), which is practice will usually mean that the γδ T cell activator is administered no more than once per year or once every 2, 3 or 6 months. In one embodiment the γδ T cell activator is administered as part of maintenance therapy conjointly with the chemotherapy or anti-angiogenic therapy. If the γδ T cell activator is administered more frequently and/or at multiple times within a course of chemotherapy treatment, the administration of successive doses of the γδ T cell activator is separated by at least 2, 3 or 4 or more weeks. Generally, the γδ T cell rate (number of γδ T cells), is allowed to return to substantially basal rate prior to a second administration of the γδ T cell activator.

When the γδ T cell activator is used with conjoint chemotherapeutic treatment, the γδ T cell activator can be administered at the end of a regimen comprising multiple doses of chemotherapeutic agent, for example within about two to eight weeks after the last dose of the chemotherapeutic agent. In one example, the γδ T cell activator can be used conjointly with a chemotherapeutic treatment such that the γδ T cell activator is be administered between two doses of a multiple dose chemotherapeutic regimen, for example within about two to eight weeks after the preceding dose of the chemotherapeutic agent, and at any time (but preferably at least about one or two weeks) prior to the following dose of the chemotherapeutic agent. It will be appreciated, however, that the γδ T cell activator need not be administered between each two successive doses of chemotherapeutic agent, and that successive administrations of the γδ T cell activator will be separated by at least 2, 3 or 4 or more weeks.

In another regimen, the γδ T cell activator is administered during the chemotherapeutic treatment. The γδ T cell activator can be administered for several cycles during the chemotherapeutic treatment. More preferably, the γδ T cell activator is administered for at least two cycles, or more preferably for at least three cycles.

In another regimen the γδ T cell activator and the chemotherapeutic agent are administered on the same day or simultaneously. This is the preferred regimen in particular for conjoint use with HDAC inhibitors and anti-angiogenic agents of the tyrosine kinase inhibitor class.

Thus, the course of a preferred cycle with a γδ T cell activator is an at least 1-weekly cycle, but more preferably at least a 2-weekly cycle (at least about 14 days), or more preferably at least 3-weekly or 4-weekly, though cycles anywhere between 2-weekly and 4-weekly are preferred. Also effective and contemplated are cycles of up to 8-weekly, for example 5-weekly, 6-weekly, 7-weekly or 8-weekly.

A subject will preferably be treated for at least two cycles, or more preferably for at least three cycles. In other aspect, treatment may continue for a greater number of cycles, for example at least 4, 5, 6 or more cycles can be envisioned. At the end of each cycle, the cycle of dosing may be repeated for as long as clinically tolerated and the tumor is under control or until tumor regression. Tumor “control” is a well recognized clinical parameter, as defined above. In a preferred embodiment, the cycle of dosing is repeated for up to about eight cycles

Various combinations may be employed, where γδ T cell activator is “A” and the chemotherapy or anti-angiogenic therapy is “B”, as exemplified below.

Combination Protocols

| | | | |
|---|---|---|---|
| A/B/A | B/B/B/A | B/A/B/A | A/A/B/A |
| B/A/B | B/B/A/B | B/A/A/B | A/B/B/B |
| B/B/A | A/A/B/B | B/B/B/A | B/A/B/B |
| A/A/B | A/B/A/B | A/A/A/B | B/B/A/B |
| B/A/A | A/B/B/A | B/A/A/A | |
| A/B/B | B/B/A/A | A/B/A/A | |

Other combinations are contemplated as well. Again, to achieve cell killing, ideally both agents are delivered to a cell in a combined amount effective to kill the cell.

Co-Treatment with Cytokine

In embodiments where the γδ T cell activator is used conjointly with a chemotherapeutic agent, the methods of the invention optionally comprise further administering a cytokine. While the compounds of the invention may be used with or without further administration, in a preferred aspect a cytokine can be administered, wherein said cytokine is capable of increasing the expansion of a γδ T cell population treated with a γδ T cell activator compound, preferably wherein the cytokine is capable of inducing an expansion of a γδ T cell population which is greater than the expansion resulting from administration of the γδ T cell activator compound in the absence of said cytokine. A preferred cytokine is an interleukin-2 polypeptide.

A cytokine having γδ T cell proliferation inducing activity, most preferably the interleukin-2 polypeptide, is administered at low doses, typically over a period of time comprised between 1 and 10 days. The γδ T cell activator is preferably administered in a single dose, and typically at the beginning of a cycle.

In preferred aspects, a cytokine, most preferably IL-2, is administered daily for up to about 10 days, preferably for a period of between about 3 and 10 days, or most preferably for about 7 days. Preferably, the administration of the cytokine begins on the same day (e.g. within 24 hours of) as administration of the γδ T cell activator. It will be appreciated that the cytokine can be administered in any suitable scheme within said regimen of between about 3 and 10 days. For example, in one aspect the cytokine is administered each day, while in other aspects the cytokine need not be administered on each day. When the cytokine is administered for about 7 to about 14 days, a 4-weekly treatment cycle is preferred. When the first component is administered for about 4 days, a 3-weekly day treatment cycle is preferred.

### Mode of Use

As disclosed herein, the γδ T cell activator is preferably administered as a single dose (“single shot”). The chemotherapeutic agent will be administered following manufacturer's instructions, or according to any other suitable protocol, for example standard protocols proven to have efficacy in the treatment of disease. When used in a treatment comprising more than one administration of the γδ T cell activator, the latter is preferably administered as a single shot at the beginning of a treatment cycle. As shown in the experimental section, such administration schedule provides a remarkable increase in the activity of γδ T cells in a subject. The active ingredients may be administered through different routes, typically by injection or oral administration. Injection may be carried out into various tissues, such as by intravenous, intra-peritoneal, intra-arterial, intra-muscular, intra-dermic, subcutaneous, etc.

Most preferably, the γδ T cell activator is administered by intravenous (i.v.) administration. Preferably said infusion is during 2 to 180 min, preferably 2 to 120 min, more preferably during about 5 to about 30 min, most preferably during about 10 to about 30 min, e.g. during about 30 min. As further described herein, the invention discloses that a brief stimulation of γδ T cell activity is sufficient to achieve the γδ T cell regulating effect. Thus, preferably where a γδ T cell activator has a short serum half-life, for example having a serum half-life of less than about 48 hours, less than about 24 hours, or less than about 12 hours, a rapid infusion is used. Said rapid infusion is preferably between about 10 minutes and 60 minutes, or more preferably about 30 minutes.

### Chemotherapeutic Agents

### Anti-Angiogenic Therapies

New blood vessel formation (angiogenesis) is a fundamental event in the process of tumor growth and metastatic dissemination. Angiogenesis is a common feature of all solid tumors: the use of anti-angiogenic compounds can thus be applied successfully for the treatment of a wide variety of solid tumors, such as but not limited to colorestal, colon, breast tumors.

The vascular endothelial growth factor (VEGF) pathway is well established as one of the key regulators of this process. The VEGF/VEGF-receptor axis is composed of multiple ligands and receptors with overlapping and distinct ligand-receptor binding specificities, cell-type expression, and function. Activation of the VEGF-receptor pathway triggers a network of signaling processes that promote endothelial cell growth, migration, and survival from pre-existing vasculature. In addition, VEGF mediates vessel permeability, and has been associated with malignant effusions. The VEGF-related gene family comprises six secreted glycoproteins referred to as VEGF-A, VEGF-B, VEGF-C, VEGF-D, VEGF-E, placenta growth factor (PlGF)-1 and -2. A number of exemplary anti-angiogenic agents acting of the VEGR pathway are known, any of which can be used in accordance with the invention, including neutralizing antibodies antisense strategies, RNA aptamers and ribozymes against VEGF or VEGF receptors (U.S. Pat. No. 6,524,583, the disclosure of which is incorporated herein by reference). Variants of VEGF with antagonistic properties may also be employed, as described in WO 98/16551, specifically incorporated herein by reference. Further exemplary anti-angiogenic agents that are useful in connection with combined therapy are listed in Table D of U.S. Pat. No. 6,524,583, the disclosure of which agents and indications are specifically incorporated herein by reference.

Compounds that have an action on VEGF expression can also be used.

Further anti-angiogenic agents can also be specific inhibitor of proteins that induce a modification of the expression of the VEGF, and thus activate angiogenesis, such as Imatinib that specifically inhibits Bcr/Abl protein production and/or activity, responsible for a modification of VEGF expression. Other receptors having a tyrosine kinase inhibitors activity can also be used to control the vascularisation of the solid tumor and obtain an anti-angiogenic effect, such as FGFR (fibroblast growth factor receptor, FGF-1,2), PDGFR (platelet derived growth factor receptor), angiopoyetins receptors (Ang-1,2), HGFR (hepatocytary growth factor receptor), ephrines receptor (Eph).

Particularly preferred anti-angiogenic agents inhibit signaling by a receptor tyrosine kinase including but not limited to VEGFR1, VEGFR-2,3 PDGFR-α, PDGFR-β, CSF-1R, RET, Flt-3, c-Kit, bcr/abl, p38 alpha and FGFR-1.

Further anti-angiogenic agents may include agents that inhibit one or more of the various regulators of VEGF expression and production, such as EGFR, flt-1, KDR HER-2, COX-2, or HIF-1α. Another preferred class of agents includes thalidomide or the analogue CC-5013 (lenalidomide Revlimib™). Another class of anti-angiogenic agent includes cilengitide (EMD 121974, integrin inhibitor), metalloproteinases (MPP) such as marinastat (BB-251), metastat (CMT-3, COL-3, Collagenex™). Another class of anti-angiogenic agents includes famesylation inhibitors such as lonafamib (Sarasar™), tipifarnib (Zamestra™). Other anti-angiogenic agents can also be suitable such as Bevacuzimab (mAb, inhibiting VEGF-A, Genentech); IMC-1121B (mAb, inhibiting VEGFR-2, ImClone Systems); CDP-791 (Pegylated DiFab, VEGFR-2, Celltech); 2C3 (mAb, VEGF-A, Peregrine Pharmaceuticals); VEGF-trap (Soluble hybrid receptor VEGF-A, PlGF (placenta growth factor) Aventis/Regeneron).

### Tyrosine Kinase Inhibitors as Anti-Angiogenic Agents

Selected tyrosine kinase inhibitor (TKI) class include PTK-787 (TKI, VEGFR-1,-2, Vatalanib, Novartis); AEE788 (TKI, VEGFR-2 and EGFR, Novartis); ZD6474 (TKI, VEGFR-1,-2,-3, EGFR, Zactima, AstraZeneca); AZD2171 (TLI, VEGFR-1,-2, AstraZeneca); SU11248 (TKI, VEGFR-1,-2, PDGFR, Sunitinib, Pfizer); AG13925 (TKI, VEGFR-1,-2, Pfizer); AG013736 (TKI, VEGFR-1,-2, Pfizer); CEP-7055 (TKI, VEGFR-1,-2,-3, Cephalon); CP-547,632 (TKI, VEGFR-1,-2, Pfizer); GW786024 (TKI, VEGFR-1,-2,-3, GlaxoSmithKline): GW786034 (TKI, VEGFR-1,-2,-3, GlaxoSmithKline); sorafenib (TKI, Bay 43-9006, VEGFR-1,-2, PDGFR Bayer/Onyx); SU4312 (° TKI, VEGFR, PDGFR, Pfizer), AMG706 (TKI, VEGFR-1,-2,-3, Amgen), XL647 (TKI, EGFR, HER2, VEGFR, ErbB4, Exelixis), XL999 (TKI, FGFR, VEGFR, PDGFR, Flt-3, Exelixis), PKC412 (TKI, KIT, PDGFR, PKC, FLT3, VEGFR-2, Novartis), AEE788 (TKI, EGFR, HER2, VEGFR, Novartis), OSI-930 (TKI, c-kit, VEGFR, OSI Pharmaceuticals), OSI-817 (TKI, c-kit, VEGFR, OSI Pharmaccuticals), DMPQ (TKI, ERGF, PDCFR, erbB2, p56, pkA, pkC), MLN518 (TKI, FLT3, PDGFR, c-KIT, CT53518, Millennium Pharmaceuticals), lestaurinib (TKI, FLT3, CEP701, Cephalon), ZD1839 (TKI, EGFR, gefitinib, Iressa, AstraZeneca), OSI-774 (TKI, EGFR, Erlotininb, Tarceva, OSI Pharmaceuticals), lapatinib (TKI, ErbB-2, EGFR, GD-2016, Tykerb, GlaxoSmithKline).

Most preferred are tyrosine kinase inhibitors that inhibit one or more receptor tyrosine kinases selected from the group consisting of VEGFR-1, VEGFR-2, VEGFR-3, PDGFR-α, β, Flt-3, c-Kit, p38 alpha, RET, c-RAF, b-RAF, bcr/abl and FGFR-1. Preferred examples include SU11248 (sunitinib, Sutent™, Pfizer), BAY 43-9006 (sorafenib, Nexavar™, Bayer) and STI-571 (Imatinib, Gleevec™). It will be appreciated that other TKI compounds inhibiting said tyrosine kinases can be used in accordance with the invention. TKI compound used in accordance with the invention may have anti-angiogenic activity but it will be appreciated that TKI compounds that do not have anti-angiogenic activity can also be used and are within the scope of the invention.

As is the case with inhibitors of Abl kinase and c-KIT, tyrosine kinase inhibitors can also be useful in the treatment of tumors characterized by aberrant, generally increased, tyrosine kinase signaling activity. Activating mutations in platelet-derived growth factor (PDGFR) family (type III) receptor tyrosine kinases are known for example, including FLT3 (e.g. the FLT3 length mutation (FLT3-LM) and FLT3D835) and c-KIT, which are common in AML patients. Other examples of mutations include fusions of the TEL (ETV6) gene to the PDGFR-β, gene, generating a fusion protein with constitutive tyrosine kinase activity and resulting in chronic myelomonocytic leukemia (CMML).

### Tyrosine Kinase Inhibitors as Abl Inhibitor Therapies

Bcr/Abl, a constitutively activated tyrosine kinase, is the oncogene that causes Philadelphia-chromosome-positive (Ph+) leukaemia. It is due to a reciprocal translocation, an exchange of genetic material, between chromosomes 9 and 22. Philadelphia chromosome or Philadelphia translocation is a specific chromosomal abnormality that is associated with chronic myelogenous leukemia (CML) and acute lymphoblastic leukemia (ALL).

Various TKI are competitive inhibitor at the ATP-binding site of Bcr/Abl and have been shown to have high activity in this type of leukaemia.

The first compound was STI-571 (Imatinib, Gleevec), identified by Novartis pharmaceuticals in high-throughput screens for tyrosine kinase inhibitors. Subsequent clinical trials demonstrated that STI-571 inhibits proliferation of Bcr/Abl-expressing hematopoietic cells. Although it did not eradicate CML cells, it did greatly limit the growth of the tumor clone and decreased the risk of the feared “blast crisis”. Other pharmacological inhibitors are being developed, which are more potent and/or are active against the emerging Gleevec/Glivec resistant Bcr/Abl clones in treated patients. The majority of these resistant clones are point-mutations in the kinase of Bcr/Abl.

Preferred tyrosine kinase inhibitors capable of inhibiting bcr/abl can be, but are not limited to: PD-166326 (TKI, bcr/abl, axitinib, Pfizer), NSC 680410 (TKI, bcr/abl, adaphostin, analog of NSC 654705), tyrphostin AG 957 (TKI, bcr/abl, also NSC 654705), AP-23464 (TKI, Bcr/Abl, Ariad), AP-234604 (TKI, Bcr/Abl, Ariad), SKI-606 (TKI, bcr/abl, Wyeth, Bosutinib), dasatinib (TKI, Src/Abl, Bristol-Myers Squibb), nilotinib (TKI, Bcr-Abl, Kit, PDGFR, AMN107, Tasigna™, Novartis), tyrphostin AG 957 (TKI, bcr/abl, also NSC 654705), NS-187 (bcr/abl, also referred to as CNS-9 and INNO-406, Nippon Shinyaku), CGP16030 (TKI, bcl/abl, Novartis).

Some of the preferred tyrosine kinase inhibitors are TKI that can be even administered once the patient has become resistant to the initial treatment (e.g. with imatinib), examples of such products include for example dasatinib (BMS-354825, Spryce™) which inhibits KITD816V, an imatinib-resistant activating mutation (Shah et al. Blood, Jul. 1, 2006; 108(1): 286 -291.), nilotinib (AMN107, Tasigna™) which possesses substantially increased binding affinity and selectivity for the Abl kinase compared with imatinib.

The aforementioned TKI have also been shown to inhibit KIT which can be mutated in GIST. In many cases GISTs are characterized by gain of function mutations in the c-kit proto-oncogene, which result in constitutive activation of the KIT tyrosine kinase transmembrane receptor (KIT).

Such tyrosine kinases inhibiting Bcr/Abl kinase treatments have been shown to be efficient, but in some cases, the treatment does not provide a complete remission and a residual disease persists. In the treatment of CML, classical TKI therapies (e.g. imatinib) can reduce the residual disease to 10⁴ gene modification but cannot maintain the residual disease to a level below the detection limit. (Deininger et al. 105 (7): 2640, Blood 2005). The detection limit of the residual disease dosed with PCR gene amplification, is the detection of a gene modification out of 10⁵ standard genes (Barbany et al. 46 (7): 913. Clin Chem 2000). In GIST for example, correlative studies may include immunochemical expression of CD117 (a marker for KIT), A further object of the invention is to provide a treatment enabling to diminish the residual disease to a level lower than the detection limits, leading to a cure or a stabilization of the disease.

Preferred examples include imitanib mesylate (ST1571, Glivec™, Gleevec™, Novartis), dasatinib (TKI, Src/Abl, Bristol-Myers Squibb) and nilotinib (TKI, Bcr-Abl, Kit, PDGFR, AMN107, Tasigna™, Novartis),

Encompassed is a method of treatment of diseases, preferably cancer, preferably CML or ALL, where the treatment with an antiangionenic agent or a TKI together with a γδ T cell activator maintains the residual disease below the detection limit as established using PCR gene amplification technique.

Encompassed is a method of treatment of diseases, preferably cancer, preferably CML or ALL, where the treatment with a TKI inhibiting signaling by receptor tyrosine kinases including but not limited to bcr/abl and receptors of the same family such as c-Kit and PDGFR together with a γδ T cell activator maintains the residual disease below the detection limit as established using PCR gene amplification technique.

Encompassed is a method of treatment of diseases, preferably cancer, preferably CML or ALL, where the treatment with imatinib together with a γδ T cell activator maintains the residual disease below the detection limit as established using PCR gene amplification technique.

More generally, TKI are also known to induce many undesirable side effects, such as rash/desquamation, hand-foot skin reaction, fatigue, diarrhea, nausea, stomatitis, erectile dysfunction, alopecia, headache, hypertension, asthenia, anorexia. TKI also induce other side effects such as lymphopenia, neutropenia, anemia, leucopenia, thrombocytopenia. This immunosuppressant effect, weakening the immune cells, is known to impair the efficacy of immunotherapies. Imatinib is particularly known to induce such effect. (Appel et al., Blood 2004, Dietz et al., Blood 2004, Seggewiss et al., Blood 2005). Sorafenib has also been reported to induce an immunosuppressant effect, as highlighted by the drug's prescribing information. Sunitinib has also been reported to induce an immunosuppressant effect.

The inventors have found out that the proliferative effect of γδ T cell activators according to the invention are not impaired by a TKI co-administration.

The invention thus provides a treatment regimen combining one or more TKIs and a γδ T cell activator, wherein the TKI is administered in an effective amount such that the TKI does not significantly impair the patient's γδ T cell proliferative response to treatment with the γδ T cell activator.

TKI treatments also raise toxicology issues and a combination treatment with TKI is usually avoided as long as such combination could lead to a combination or an enhancement of the toxicity of each drug taken separately, which could be dangerous or even fatal to the patient.

The inventors have found out that a combination of a γδ T cell activating agent with a TKI did not lead to an increase in toxicity compared to a treatment with TKI alone. A further object of the invention is thus to provide a treatment with a toxicity of an acceptable level, compared with existing treatments and monotherapies.

Encompassed is the conjoint use of a composition comprising a TKI which inhibits signaling by one or more receptor tyrosine kinase(s) selected from the group consisting of VEGFR1, VEGFR-2,3, PDGFR-beta, Flt-3, and c-Kit, and a γδ T cell activator with a toxicity not higher than the toxicity of the TKI alone. Encompassed is a composition comprising a TKI, in particular sorafenib and a γδ T cell activator with a toxicity not higher than the toxicity of the TKI alone.

Encompassed is the conjoint use of a composition comprising a TKI which inhibits signaling by one or more receptor tyrosine kinase(s) selected from the group consisting of VEGFR-1,2,3, CSF-1R, PDGFR-α,β, Flt-3, c-Kit, RET and a γδ T cell activator with a toxicity not higher than the toxicity of the TKI alone. Encompassed is a composition comprising a TKI, in particular sunitinib and a γδ T cell activator with a toxicity not higher than the toxicity of the TKI alone.

Encompassed is the conjoint use of a composition comprising a TKI which inhibits signaling by one or more receptor tyrosine kinase(s) selected from the group consisting of bcr/abl and receptors of the same family such as c-Kit and PDGFR, and a γδ T cell activator with a toxicity not higher than the toxicity of the TKI alone. Encompassed is a composition comprising a TKI, in particular imatinib and a γδ T cell activator with a toxicity not higher than the toxicity of the TKI alone.

Particularly preferred anti-angiogenic agents show a similar inhibitory pattern to sorafenib and inhibit signaling by receptor tyrosine kinases including but not limited to VEGFR-1, VEGFR-2, VEGFR-3, PDGFR-β, Flt-3, c-Kit.

Particularly preferred anti-angiogenic agents show a similar inhibitory pattern to sunitinib and inhibit signaling by a receptor tyrosine kinase including but not limited to VEGFR-1, VEGFR-2, VEGFR-3, CSF-1R, PDGFR-α,β, Flt-3, c-Kit, RET.

Particularly preferred anti-angiogenic agents show a similar inhibitory pattern to imatinib and inhibit signaling by a receptor tyrosine kinase including but not limited to bcr/abl and receptors of the same family such as c-Kit and PDGFR.

A variety of cancers and other proliferative diseases including, but not limited to, the following can be treated using the methods and compositions of the invention, preferably the cancer to be treated is renal cell carcinoma (RCC), metastatic renal cell carcinoma (mRCC), glioblastoma, gastrointestinal stromal tumors (GIST), chronic myeloid leukemia (CML), acute lymphoblastic leukemia (ALL).

Encompassed is a method to treat mRCC or RCC using a TKI inhibiting signaling by receptor tyrosine kinases including but not limited to VEGFR1, VEGFR-2,3, PDGFR-beta, Flt-3, c-Kit in combination with a γδ T cell activator.

Encompassed is a method to treat mRCC or RCC using sorafenib in combination with a γδ T cell activator.

Encompassed is a method to treat mRCC, RCC or GIST using a TKI inhibiting signaling by receptor tyrosine kinases including but not limited to VEGFR-1,2,3, CSF-1R, PDGFR-α,β, Flt-3, c-Kit, RET in combination with a γδ T cell activator.

Encompassed is a method to treat mRCC, RCC or GIST using sunitinib in combination with a γδ T cell activator.

Encompassed is a method to treat CML, ALL or GIST using a TKI inhibiting signaling by receptor tyrosine kinases including but not limited to bcr/abl and receptors of the same family such as c-Kit and PDGFR in combination with a γδ T cell activator.

Encompassed is a method to treat CML, ALL or GIST using imatinib in combination with a γδ T cell activator.

Applicants have shown in the examples that coadministration of such TKI, in particular sunitinib, sorafenib and imatinib with a γδ T cell activator does not impair the ability of γδ T cells to proliferate. It will be appreciated from the examples that other TKI showing the same inhibition pattern will have the same effect on coadministration.

Encompassed is a method to treat a proliferative disease or a cancer using a composition comprising a TKI inhibiting signaling by receptor tyrosine kinases including but not limited to VEGFR1, VEGFR-2,3, PDGFR-beta, Flt-3, c-Kit and a γδ T cell activator, wherein the administration of the TKI does not impair the proliferation of γδ T cells.

Encompassed is a method to treat a proliferative disease or a cancer using a composition comprising sorafenib and a γδ T cell activator, wherein the administration of sorafenib does not impair the proliferation of γδ T cells.

Encompassed is a method to treat a proliferative disease or a cancer using a composition comprising a TKI inhibiting signaling by receptor tyrosine kinases including but not limited to VEGFR-1,2,3, CSF-1R, PDGFR-α,β, Flt-3, c-Kit, RET and a γδ T cell activator, wherein the administration of the TKI does not impair the proliferation of γδ T cells.

Encompassed is a method to treat a proliferative disease or a cancer using a composition comprising sunitinib and a γδ T cell activator, wherein the administration of sunitinib does not impair the proliferation of γδ T cells.

Encompassed is a method to treat a proliferative disease or a cancer using a composition comprising a TKI inhibiting signaling by receptor tyrosine kinases including but not limited to bcr/abl and receptors of the same family such as c-Kit and PDGFR and a γδ T cell activator, wherein the administration of the TKI does not impair the proliferation of γδ T cells.

Encompassed is a method to treat a proliferative disease or a cancer using a composition comprising imatinib and a γδ T cell activator, wherein the administration of imatinib does not impair the proliferation of γδ T cells.

Particularly preferred anti-angiogenic agents show a similar inhibitory pattern to sorafenib and inhibit signaling by receptor tyrosine kinases including but not limited to VEGFR1, VEGFR-2,3, PDGFR-beta, Flt-3, c-Kit.

Particularly preferred anti-angiogenic agents show a similar inhibitory pattern to sunitinib and inhibit signaling by a receptor tyrosine kinase including but not limited to VEGFR-1,2,3, CSF-1R, PDGFR-α,β, Flt-3, c-Kit, RET.

Particularly preferred anti-angiogenic agents show a similar inhibitory pattern to imatinib and inhibit signaling by a receptor tyrosine kinase including but not limited to bcr/abl and receptors of the same family such as c-Kit and PDGFR.

In another aspect of the invention, the chemotherapeutic agent is not imatinib (STI571, Glivec™, Gleevec™, Novartis).

In another aspect of the invention, the chemotherapeutic agent is not SU11248 (sunitinib, Sutent™, Pfizer).

In another aspect of the invention, the chemotherapeutic agent is not Bay 43-9006 (sorafenib, Nexavar™, Bayer).

In the framework of the invention, imatinib refers to all acceptable salts and derivatives, including imatinib mesylate.

In the framework of the invention, sunitinib refers to all acceptable salts and derivatives, including sunitinib malate.

In the framework of the invention, sorafenib refers to all acceptable salts and derivatives, including sorafenib tosylate.

### Ras and raf Kinase Inhibitors

Several important signaling elements of the MAPK pathway, particularly Ras and Raf, are encoded by oncogenes, and as such, their structures and functions can be modified, rendering them constitutively active. Because the MAPK pathway is dysregulated in a notable proportion of human malignancies, many of its aberrant and critical components represent strategic targets for therapeutic development against cancer.

Raf, which is an essential serine/threonine kinase constituent of the MAPK pathway and a downstream effector of the central signal transduction mediator Ras, is activated in a wide range of human malignancies by aberrant signaling upstream of the protein (e.g., growth factor receptors and mutant Ras) and activating mutations of the protein itself, both of which confer a proliferative advantage.

Three isoforms of Raf have been identified, including A-Raf, B-Raf and C-Raf, reviewed in Beeram et al. (J. Clin. Oncol. (2005) 23:6771-90). The Ser/Thr kinase Raf-1 is a protooncogene product that is a central component in many signaling pathways involved in normal cell growth and oncogenic transformation. Upon activation, Raf-1 phosphorylates mitogen-activated protein kinase (MEK), which in turn activates mitogen-activated protein kinase/extracellular signal-regulated kinases (MAPK/ERKs), leading to the propagation of signals. Depending on specific stimuli and cellular environment, the Raf-1-MEK-ERK cascade regulates diverse cellular processes such as proliferation, differentiation, and apoptosis. Raf-1 also acts via a MEK-ERK-independent prosurvival mechanism; Raf-1 interacts with the proapoptotic, stress-activated protein kinase ASK1 (apoptosis signal-regulating kinase 1) in vitro and in vivo. C-Raf, in particular has been shown to have antiapoptotic effects mediated by a mitochondrial pool of C-Raf which upon stimulation interacts with proapoptotic proteins and abrogates their proapoptotic effect.

Chemotherapeutic agents targeting Raf, including small-molecule inhibitors and antisense oligodeoxyribonucleotides, are undergoing clinical evaluation. The outcomes of these investigations may have far-reaching implications in the management of many types of human cancer. Agents may inhibit one Raf enzyme (e.g. A-Raf, B-Raf or C-Raf) or a plurality of enzymes. One example is the small molecule compound sorafenib (BAY 43-9006, Bayer, Germany) which inhibits multiple Raf isoforms as well as other kinases, such that not only the MAPK pathway is inhibited but also VEGFR-2,3 PDGFR-beta, Flt-3, c-Kit, p38 alpha and FGFR-1. Other small molecule competitive inhibitors of Raf include L-779450 (Merck Pharmaceuticals, Nutley, N.J.), phenol substituted oxindole derivative SB203580 (GSK, Philadelphia, Pa.). Another example of a Raf inhibitor is Isis 5132 (CGP 69846A), an antisense oligonucleotide inhibitor of C-Raf, which depends on cellular uptake of the oligonucleotide and results in RNAase H mediated degradation of the Raf mRNA-oligonucleotide complex. Other examples include agents that indirectly inhibit Raf, including inhibitors of chaperon proteins such as geldanamycin analogs (e.g. 17-allylamino-17-demethoxygeldanamycin) which destabilize Raf (at least C-Raf interacts with chaperone proteins HSP70 and HSP90 which modulate its kinase activity). Other examples include agents such as HDAC inhibitors which reduce Raf expression (Yu et al. (2002) J. Natl. Cancer Inst. 94:504-513). For example depsipeptide FR901228 was reported to reduce Raf expression. Other example include tyrosine kinase inhibitors that inhibit multiple angiogenic factors in addition to ras and Raf, such as VEGF, PDGF, TGF-alpha induced signaling through receptor tyrosine kinases.

### Further Selected Chemotherapeutic Agents

Further preferred chemotherapeutic agents that can be used for conjoint therapy with γδ T cell activators include for example ionizing radiation or UV radiation and chemotherapeutic agents selected from the group consisting of inhibitors of DNA replication, chromatin modifying treatments, and inducers of apoptosis. Preferably, chemotherapeutic agents are administered at standard or even at low doses, for example as chronic low-dose or metronomic therapy; chemotherapeutic agent are preferably administered at less than high-dose chemotherapy (e.g. at MTD (maximum tolerated dose)) and in particular below doses requiring bone marrow rescue.

In one embodiment, the agent is a chemotherapeutic agents or radiation that upregulate expression of NKG2D ligands on the surface of tumor cells. These include well known chemotherapies including ionizing and UV radiation, inhibitors of DNA replication, inhibitors of DNA polymerase, chromatin modifying treatments, as well as apoptosis inducing agents such as HDAC inhibitors trichostatin A and valproic acid, and agents that activate an ATR or ATM protein kinase. NKG2D is an activating receptor that interacts with the MHC class I-related MICA and MICB glycoproteins, among other ligands. MICA and MICB (Bauer et al. (1999) Science 285:727-729, the disclosure of which is incorporated herein by reference) have no role in antigen presentation, are generally only found in intestinal epithelium, and can be stress-induced in permissive types of cells by viral and bacterial infections, malignant transformation, and proliferation. NKG2D is a C-type lectin-like activating receptor that signals through the associated DAP10 adaptor protein, which is similar to CD28. It is expressed on most natural killer (NK) cells, NKT cells, γδ T cells CD8 T cells, and T cells, but not, in general, on CD4 T cells. Ligand engagement of NKG2D activates NK cells and potently co-stimulates effector T cells, however certain NKG2D ligands also induce potent inhibition of proliferation (Kriegeskorte et al. (2005) PNAS 102(33): 11805-11810). Expression of NKG2D in NK cells is controlled by ligand-induced down-modulation, which is transient and rapidly reversed in the presence of IL-15. Other NKG2D ligands include ULBP proteins, e.g., ULBP-1,-2, and -3, originally identified as ligands for the human cytomegalovirus glycoprotein UL16 (Cosman et al, (2001) Immunity 14: 123-133, the disclosure of which is incorporated herein by reference). These proteins are distantly related to MHC class I proteins, but they possess only the a1 and a2 Ig-like domains, and they have no capacity to bind peptide or interact with b2-microglobulin. Further NKG2D ligands include RAE1TG, a member of the ULBP-like family ofproteins (Bacon et a1 (2004) J. Immunol. 173:1078-1084) and Letal (PCT patent publication no. WO 2004/022706, both of the foregoing disclosure incorporated herein by reference.

Thus, in one aspect, the invention provides a method for activating and/or inducing the proliferation of γδ T cells in a mammal, the method comprising conjointly administering to the mammal a γδ T cell activator and a chemotherapeutic agent capable of inducing the expression of an NKG2D ligand on the surface of a tumor cell. Preferably, the chemotherapeutic agent is ionizing radiation or UV radiation, or a chemotherapeutic agent selected from the group consisting of inhibitors of DNA replication, chromatin modifying treatments, and inducers of apoptosis, as well as agents that activate an ATR or ATM protein kinase or CHK1. In another aspect the invention encompasses a method for killing or inhibiting a proliferating cell, preferably a tumor cell in a mammal, the method comprising conjointly administering to the mammal a γδ T cell activator and a chemotherapeutic agent is capable of inducing the expression of an NKG2D ligand on the surface of a tumor cell. It will be appreciated that the method may also encompass a step of determining, prior to treatment with γδ T cell activator or prior to both chemotherapy and γδ T cell activation, whether the tumor of an individual expresses an NKG2D ligand, or whether the tumor can be induced (e.g. by a chemotherapeutic agent) to express an NKG2D ligand.

To treat a tumor or cancer, kill cells, inhibit cell growth, inhibit metastasis, or otherwise reverse or reduce the malignant phenotype of cancer cells, using the methods and compositions of the present invention, one would generally administer to a subject a γδ T cell activator in combination with a chemotherapeutic agent such as taxol, doxorubicin, radiotherapy or other agent. Preferred therapies are those that activate the DNA damage response pathway, more preferably those that activate the ATM (ataxia telangiectasia, mutated) or ATR (ATM- and Rad3-related) protein kinases, or CHK1, or yet further CHK2 or p53. Examples of the latter include ionizing radiation, inhibitors of DNA replication, DNA polymerase inhibitors and chromatic modifying agents or treatment including HDAC inhibitors. All of these compositions would be provided in a combined amount effective to kill or inhibit proliferation of the cell yet permit strong proliferation of γδ T cells. Compositions that upregulate NKG2D ligands are further described in Gasser et al (2005) Nature 436(7054):1186-90.

As further described, suitable chemotherapeutic compounds or methods for use in accordance with the invention include alkylating agents, cytotoxic antibiotics such as topoisomerase I inhibitors, topoisomerase II inhibitors, plant derivatives, RNA/DNA antimetabolites, and antimitotic agents. Preferred examples may include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, taxol, gemcitabine, navelbine, transplatinum, 5-fluorouracil, vincristin, vinblastin and methotrexate, or any analog or derivative variant of the foregoing.

Alkylating agents are substances that form compounds that are highly chemically reactive and rapidly form covalent bonds with suitable substances. One such target is DNA, not in its normal state but when the double helix has been unpaired by helicases. This exposes the ‘inside’ of the DNA, which is susceptible to alkylation. Most alkylating agents are bipolar, i.e., they contain two groups capable of reacting with DNA. They can thus form ‘bridges’ between two parts of a single strand of DNA or two separate strands; either way, this interferes with the actions of the enzymes involved with the replication process, which are unable to complete their effects. The cell then either dies because it is physically unable to divide or because the abnormal DNA stimulates apoptosis. Examples include nitrogen mustards (e.g. chlorambucil, cyclophosphamide), nitrosureas (e.g. carmustine, lomustine), metal salts (e.g. cisplatin, carboplatin, oxaliplatin), ethylenamine derivatives (e.g. thiotepa), alkyl sulphonates (e.g. busulphan) and triazenes (e.g. dacarbazine).

Antimetabolites are a group of chemicals that are similar in structure or function to naturally occurring metabolites required for the synthesis of nucleic acids. Antimetabolite molecules mimic these normal metabolites and either block the enzymes responsible for nucleic acid synthesis or become incorporated into DNA, which produces an incorrect genetic code and leads to apoptosis. There are three main classes of antimetabolites. Folate is a substance that is necessary for the synthesis of purine molecules. Folate analogues (e.g. methotrexate, raltritrexed) are similar to the folate molecule—substances such as methotrexate can be used to inhibit the enzyme dihydrofolate reductase, resulting in insufficient production of the purine thymine. Pyrimidine analogues (e.g. cytarabine, fluoroacil (5-FU), gemcitabine) resemble pyrimidine molecules and work by either inhibiting the synthesis of nucleic acids (e.g. fluorouracil) or by becoming incorporated into DNA (e.g. cytarabine). Purine analogues (e.g. mercaptopurine, thioguanine, cladribine, fludarabine) work in similar ways to pyrimidine analogues, but may have additional (and ill-characterized) mechanisms of action.

Cytotoxic antibiotics are so called because they are all derived from a natural source, the Streptomyces group of bacteria. They affect the function and synthesis of nucleic acids in different ways. The anthracycline group includes doxorubicin, daunorubicin and idarubicin. They intercalate with DNA and affect the topoisomerase TI enzyme. This DNA gyrase splits the DNA double helix and reconnects it once torsional forces have been relieved; the anthracyclines stabilize the DNA-topoisomerase TI complex and thus prevent reconnection of the strands. Dactinomycin and mitoxantrone have a similar mechanism of action. Bleomycin causes fragmentation of DNA chains. Mitomycin functions similar to the alkylating agents, causing DNA cross-linkage.

Plant Derivatives include the vinca alkaloids such as vincristine and vinblastine bind to precursors of microtubules, preventing their formation. This inhibits the process of mitosis. The taxanes (paclitaxel and docetaxel) also act on microtubules. They stabilize them in their polymerized state, which also causes the arrest of mitosis. Podophyllyum derivatives such as etoposide and teniposide are thought to inhibit topoisomerase II, while irinotecan and topotecan inhibit topoisomerase I.

Inhibitors of topoisomerase (Topo) I and Topo II. One preferred group of compounds are Topo inhibitors. The majority of Topo inhibitors interfere with the religation step in the normal action of the enzymes, which leads to a stabilization of cleavable Topo-DNA complexes. This produces single-strand DNA breaks in the case of Topo I or double-strand breaks in the case of Topo II. Single-strand breaks caused by Topo I inhibition are converted into double-strand breaks in the course of DNA replication. Examples of compounds include camptothecin (a Topo I poison) and etoposide, adriamycin and genistein (Topo II poisons). While the role of ATM in DNA damage responses to Topo inhibitors is not conclusive, activation of downstream mediators of the DNA damage response pathway p53 and CHK2 by Topo II inhibitors adriamycin, etoposide and genistein is observed (genisteam activation being ATM dependent, and adriamycin, etoposide activation ATM independent). Both topo I inhibitors topotecan and topo II inhibitor mitoxantrone have been reported to activate ATM (Kurose et al., Cytometry A. 2005).

Taxol/Paclitaxel. Another preferred group of compounds are taxanes. Paclitaxel, also known as taxol is a diterpene alkaloid thus it possesses a taxane skeleton in its structure. Paclitaxel is extracted from the bark of the Pacific yew (Taxus brevifolia) as a natural compound having anti-cancer activity. Paclitaxel works against cancer by interfering with mitosis. Paclitaxel is a taxoid drug, widely used as an effective treatment of primary and metastatic cancers.

Paclitaxel (Taxol) is widely used in the treatment of breast, ovarian, and other solid tumors. Randomized clinical trials have shown a survival advantage among patients with primary breast cancer who received paclitaxel in addition to anthracycline-containing adjuvant chemotherapy. Furthermore, paclitaxel is effective for both metastatic breast cancer and advanced ovarian cancer. The antitumor activity of paclitaxel is unique because it promotes microtubule assembly and stabilizes the microtubules, thus preventing mitosis. Paclitaxel does this by reversibly and specifically binding to the B subunit of tubulin, forming microtubule polymers thereby stabilizing them against depolymerization and thus leading to growth arrest in the G2/M phase of the cell cycle. This makes taxol unique in comparison to vincristine and vinblastine which cause microtubule disassembly. Additionally, recent evidence indicates that the microtubule system is essential to the release of various cytokines and modulation of cytokine release may play a major role in the drug's antitumor activity.

However, some patients are resistant to paclitaxel therapy, and the characteristics of patients who will benefit from the drug have not been well defined. Identification of molecular characteristics predictive of paclitaxel sensitivity or resistance could aid in selecting patients to receive this therapy. Thus, in particular embodiments, the present invention relates to paclitaxel sensitivity in a patient having cancer. Previous reports have demonstrated that paclitaxel resistance is due to a variety of mechanisms such as up-regulation of anti-apoptotic Bcl-2 family members, such as Bel-2 and BCl-XL; up-regulation of membrane transporters (e.g., mdr-1), resulting in an increased drug efflux; mutations in beta-tubulin resulting in abolishment of paclitaxel binding; and up-regulation of ErbB2 (HER2) through inhibition of cyclin-dependent kinase-1 (Cdk1), resulting in delayed mitosis.

Due to the antimitotic activity of paclitaxel it is a useful cytotoxic drug in treating several classic refractory tumors. Paclitaxel has primarily been use to treat breast cancer and ovarian cancer. It may also be used in treating head and neck cancer, Kaposi's sarcoma and lung cancer, small cell and non-small cell lung cancer. It may also slow the course of melanoma. Response rates to taxol treatment vary among cancers. Advanced drug refractory ovarian cancer is reported to respond at a 19-36% rate, previously treated metastatic breast cancer at 27-62%, and various lung cancers at 21-37%. Taxol has also been shown to produce complete tumor remission in some cases.

Paclitaxel is given intravenously since it irritates skin and mucous membranes on contact. It is typically administered intravenously by a 3 to 24 hour infusion three times per week.

Many analogs of taxol are known, including taxotere, the structure of which is shown in FIG. 4 of PCT patent publication no. WO 03/006430, the disclosure of which is incorporated by reference. Taxotere is also referred to as “Docetaxol”. The structure of taxol and other taxol analogs, as well as dosages are shown in FIGS. 5-25 of PCT patent publication no. WO 03/006430. These compounds have the basic taxane skeleton as a common structure feature and have also been shown to have the ability to arrest cells in the G2-M phases due to stabilized microtubules. Thus, it is apparent that a wide variety of substituents can decorate the taxane skeleton without adversely affecting biological activity. Such taxol analogs or taxane comprising compounds are thus within the scope of the invention.

Doxorubicin. Doxorubicin hydrochloride, 5,12-Naphthacenedione, (8s-ds)-10-[(3-amino-2,3,6-trideoxy-α-L-Iyxo-hexopyranosyl)oxy]-7,8,9,10-tetrahydro-6,8,11-trihydroxy-g-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis and mitosis, and promotes chromosomal aberrations.

Administered alone, it is the drug of first choice for the treatment of thyroid adenorna and primary hepatocellular carcinoma. It is a component of first-choice in combination with other agents for the treatment of ovarian tumors, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myelonia, diffuse histiocytic lymphoina, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Since doxorubicin is poorly absorbed it is administered intravenously. The pharmacokinetics of this chemotherapeutic agent are multicompartmental. Distribution phases have half-lives of 12 minutes and 3.3 hrs. The elimination half-life is about 30 hrs. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, for an adult, administered intravenously, are 60 to 75 mg/m² at 21-day intervals, or 25 to 30 mg/m² on each of 2 or 3 successive days repeated at 3- or 4-week intervals, or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirabin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days repeated every 4 weeks may be administered. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 nig/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the present invention.

Radiotherapy. Radiotherapy, also called radiation therapy, is another preferred chemotherapeutic and involves the use of ionizing radiation to treat cancers and other diseases. Ionizing radiation deposits energy that injures or destroys cells in the area being treated (the “target tissue”) by damaging their genetic material, and thereby inhibiting cell proliferation. Ionizing radiation induces the formation of hydroxyl radicals, placing the cells under oxidative stress. These radicals damage DNA, which causes cytotoxicity.

Radiotherapeutic agents that cause DNA damage are well known in the art and have been extensively used. Radiotherapeutic agents, through the production of oxygen-related free radicals and DNA damage, may lead to cell death or apoptosis. These agents may include, but are not limited to, 7-rays, X-rays, and/or the directed delivery of radioisotopes to tumor cells (known as internal radiotherapy). Internal radiotherapy may further include but is not limited to, brachytherapy, interstitial irradiation, and intracavitary irradiation. Other radiotherapeutic agents that are DNA damaging factors include microwaves and UV-irradiation. These factors effect a broad range of damage on DNA, on the precursors of DNA, on the replication and repair of DNA, and on the assembly and maintenance of chromosomes. Other approaches to radiation therapy are also contemplated in the present invention.

Such techniques may comprise intraoperative irradiation, in which a large dose of external radiation is directed at the tumor and surrounding tissue during surgery; and particle beam radiation therapy which involves the use of fast-moving subatomic particles to treat localized cancers. Radiotherapy may further involve the use of radiosensitizers and/or radioprotectors to increase the effectiveness of radiation therapy. Radiolabeled antibodies may also be used to deliver doses of radiation directly to the cancer site, this is known as radioimmunotherapy.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Histone modification enzyme inhibitors. Further preferred chemotherapeutic agents are those that inhibit enzymes involved in the control of histone modification, including histone methyltransferases and Aurora kinases. Inhibitors for class I, II and III histone deacetylases are an emerging class of anticancer agents. They induce hyperacetylation in chromatin usually resulting in activation of certain genes. They induce terminal cell differentiation and/or apoptosis in cancer cells. Histone deacetylase (HDAC) activity is recruited by co-repressor proteins to certain regions of the chromatin and aberrant histone acetylation caused by that recruitment is responsible for the pathogenesis of certain cancers on a molecular level. The best studied inhibitor of HDAC is trichostatin A, a hydroxamic acid that exerts its activity by complexation of a zinc ion that is supposed to mediate the acetamide cleavage at the catalytic site, and trapoxin (TPX), both of which are inhibitors of the eukaryotic cell cycle and inducers of morphological reversion of transformed cells, inhibit histone deacetylase (HDAC) at nanomolar concentrations. There are several synthetic hydroxamic acids that bear resemblance to trichostatin. Another class of potent inhibitors is naturally occurring and synthetic cyclotetrapeptides that all contain an unusual amino acid with an epoxyketone, ketone or hydroxamic acid function in the side chain. Phenylacetate, phenylbutyrate, butyrate and similar short chain fatty acids are also weak inhibitors. Further inhibitors from natural sources are the epoxide depudecin and depsipeptide FR 901228. The benzamide MS-275 belongs to a new class of synthetic HDAC inhibitors and displays oral activity in animal models. Both trichostatin A and valproic acid (VPA) have also been suggested to be capable of upregulating the expression of NKG2D ligands (Gasser et al. Nature (2005) 435:1186; and Armeanu et al. Cancer Res. (2005) 65(14):6321-9). Autora kinase inhibitors are well known, reviewed in Mortlock et al., Curr Top Med Chem. 2005;5(8):807-21 and Andrews Oncogene. 28 Jul. 2005;24(32):5005-15, and Fancelli et al., J Med Chem. 21 Apr. 2005;48(8):3080-4, the disclosures of which are incorporated herein by reference.

Exemplary HDAC inhibitors thus include but are not limited to Valproic acid (VPA), sodium butyrate, Suberoylanilide hydroxamic acid (SAHA) HA-But, an HDAC inhibitor in which butyric acid residues are esterified to a hyaluronic acid backbone and characterized by a high affinity for the membrane receptor CD44, Depsipeptide FR-901228 (FK228) is a histone deacetylase inhibitor under development by Fujisawa and the National Cancer Institute, FK228 (Gloucester Pharmaceuticals), hydroxamic acids, with hydroxamic acid moiety replacement with an alpha-ketoamide moiety. HDAC inhibitors are also described in U.S. Pat. No. 6,541,661 entitled “Inhibitors of Histone Deacetylase”, including the HDAC inhibitor, MGCD0103 (MethylGene, Canada), and U.S. Pat. No. 6,888,027 and European Patent no. EP 1 301 184 including PXD101 (TopoTarget A/S, Denmark, and Curagen Inc.). Other HDAC inhibitors can be identified using assays known in the art; exemplary protocols for high-throughput assays for NAD(+)-dependent (class III) histone deacetylases are provided in Haltweg et al. Methods. (2005) 36(4):332-7; and Wegener et al. Mol Genet Metab. (2003) 80(1-2):138-47), the disclosures of which are incorporated herein by reference.

While exemplary regiments are further provided herein, preferred treatment regimens will use conjoint γδ T cell activator and HDAC inhibitor for the treatment of solid tumors. In other aspects haematological tumors are treated; for example use of a γδ T cell activator and FK228 (Gloucester Pharmaceuticals) for the treatment of peripheral T-cell lymphoma and cutaneous T-cell lymphoma; or use of a γδ T cell activator and SAHA compound (Merck & Co.) for the treatment of lymphocytic leukaemia and androgen independent prostate cancer, as well as peripheral T-cell lymphoma and cutaneous T-cell lymphoma. In a preferred embodiment, a γδ T cell activator is used conjointly with an HDAC inhibitor (e.g. PDX-101) and further with another chemotherapeutic agent (e.g. 5-Fu) for the treatment of a solid tumor (e.g. colorectal cancer).

### γδ T Cell Activators

The term “γδ T cell activator” designates a molecule, preferably artificially produced, which can activate γδ T lymphocytes. It is more preferably a ligand of the T receptor of γδ T lymphocytes. The activator may by of various natures, such as a peptide, lipid, small molecule, etc. It may be a purified or otherwise artificially produced (e.g., by chemical synthesis, or by microbiological process) endogenous ligand, or a fragment or derivative thereof, or an antibody having substantially the same antigenic specificity.

A phosphoantigen that is a γδ T cell activator preferably increases the biological activity or causes the proliferation of γδ T cells, preferably increasing the activation of γδ T cells, particularly increasing cytokine secretion from γδ T cells or increasing the cytolytic activity of γδ T cells, with or without also stimulating the proliferation or expansion of γδ T cells. Accordingly, the γδ T cell activator is administered in an amount and under conditions sufficient to increase the activity γδ T cells in a subject, preferably in an amount and under conditions sufficient to increase cytokine secretion by γδ T cells and/or to increase the cytolytic activity of γδ T cells. Cytokine secretion and cytolytic activity can be assessed using any appropriate in vitro assay.

In any exemplary assay, cytokine secretion can be determined according to the methods described in Espinosa et al. (J. Biol. Chem., 2001, Vol. 276, Issue 21, 18337-18344), describing measurement of TNF-α release in a bioassay using TNF-α-sensitive cells. Briefly, 10⁴ γδ T cells/well were incubated with stimulus plus 25 units of IL2/well in 100 μl of culture medium during 24 h at 37° C. Then, 50 μl of supernatant were added to 50 μl of WEHI cells plated at 3×10⁴ cells/well in culture medium plus actinomycin D (2 μg/ml) and LiCl (40 mM) and incubated for 20 h at 37° C. Viability of the TNF-α-sensitive cells and measured with a 3-(4,5-dimethylthiazol-2-yl)-2,5-diphenyltetrazolium bromide assay. 50 μl of 3-(4,5-dimethylthiazol-2-yl)-2,5-diphenyltetrazolium bromide (Sigma; 2.5 mg/ml in phosphate-buffered saline) per well were added, and after 4 h of incubation at 37° C., 50 μl of solubilization buffer (20% SDS, 66% dimethyl formamide, pH 4.7) were added, and absorbance (570 nm) was measured. Levels of TNF-α release were then calculated from a standard curve obtained using purified human rTNF-α (PeproTech, Inc., Rocky Hill, N.J.). Interferon-γ released by activated T cells was measured by a sandwich enzyme-linked immunosorbent assay. 5×10⁴ γδ T cells/well were incubated with stimulus plus 25 units of IL2/well in 100 μl of culture medium during 24 h at 37° C. Then, 50 μl of supernatant were harvested for enzyme-linked immunosorbent assay using mouse monoclonal antibodies (BIOSOURCE, Camarillo, Calif.).

A preferred assay for cytolytic activity is a ⁵¹Cr release assay. In exemplary assays, the cytolytic activity of γδ T cells is measured against autologous normal and tumor target cell lines, or control sensitive target cell lines such as Daudi and control resistant target cell line such as Raji in 4 h ⁵¹Cr release assay. In a specific example, target cells were used in amounts of 2×10³ cells/well and labeled with 100 μCi ⁵¹Cr for 60 minutes. Effector/Target (E/T) ratio ranged from 30:1 to 3.75:1. Specific lysis (expressed as percentage) is calculated using the standard formula

[(experimental-spontaneous release/total-spontaneous release)×100].

As discussed, the methods of the invention can generally be carried out with any γδ T cell activator that is capable of stimulating γδ T cell activity. This stimulation can be by direct effect on γδ T cells as discussed below using compounds that can stimulate γδ T cells in a pure γδ T cell culture, or the stimulation can be by an indirect mechanism, such as treatment with pharmacological agents such as bisphosphonates which lead to IPP accumulation. Preferably, a γδ T cell activator is a compound capable of regulating the activity of a γδ T cell in a population of γδ T cell clones in culture. The γδ T cell activator is capable of regulating the activity of a γδ T cell population of γδ T cell clones at millimolar concentration, preferably when the γδ T cell activator is present in culture at a concentration of less than 100 mM. Optionally a γδ T cell activator is capable of regulating the activity of a γδ T cell in a population of γδ T cell clones at millimolar concentration, preferably when the γδ T cell activator is present in culture at a concentration of less than 10 mM, or more preferably less than 1 mM. Regulating the activity of a γδ T cell can be assessed by any suitable means, preferably by assessing cytokine secretion, most preferably TNF-α secretion as described herein. Methods for obtaining a population of pure γδ T cell clones is described in Davodeau et al, (1993) and Moreau et al, (1986), the disclosures of which are incorporated herein by reference. Preferably the activator is capable of causing at least a 20%, 50% or greater increase in the number of γδ T cells in culture, or more preferably at least a 2-fold increase in the number of γδ T cells in culture.

In one embodiment, the activator may be a synthetic chemical compound capable of selectively activating Vγ9Vδ2 T lymphocytes. Selective activation of Vγ9Vδ2 T lymphocytes indicates that the compound has a selective action towards specific cell populations, preferably increasing activation of Vγ9Vδ2 T cells at a greater rate or to a greater degree than other T cell types such as Vδ1 T cells, or not substantially not activation other T cell types. Such selectivity can be assessed in vitro T cell activation assays. Such selectivity, as disclosed in the present application, suggests that preferred compounds can cause a selective or targeted activation of the proliferation or biological activity of Vγ9Vδ2 T lymphocytes.

Detection of γδ T cell proliferation can be detected by standard methods. One specific method for detecting γδ T cell amplification in vivo is described in Example 1.

### Preferred Phosphoantigens

In a preferred embodiment, the phosphoantigen is a compound of Formula I, especially a γδ T cell activator according to Formulas I to III, especially γδ T cell activator selected from the group consisting of BrHPP, CBrHPP, CHDMAPP, NHMDMAPP, HDMAPP and epoxPP. In the framework of the present invention, the expression “Formulas I to III”, designate all compounds derived from Formulas I to III: I, II, IIa, III, IIIa, IIIa1, IIIa2, IIIa3, A, B, IIIb, IIIb1, IIIb2, IIIb3, C, IIIc, IIIc1, IIIc2, IIIc3, D, E, F and G. Most preferably, the compounds are selected from the list consisting of BrHPP, CBrHPP, CHDMAPP, NHMDMAPP, HDMAPP and epoxPP. However, it will appreciated that a number of phosphoantigen compounds that are less potent γδ T cell activators are available and may be used in accordance with the invention. For example, in one variant, a bisphosphonate compounds such as pamidronate (Novartis, Nuernberg, Germany) or zoledronate may be used. Other γδ T cell activators for use in the present invention are phosphoantigens disclosed in WO 95/20673, isopentenyl pyrophosphate (IPP) (U.S. Pat. No. 5,639,653), the disclosures of the two preceding documents being incorporated herein by reference, as well as alkylamines (such as ethylamine, iso-propyulamine, n-propylamine, n-butylamine and iso-butylamine, for instance). Isobutyl amine and 3-aminopropyl phosphonic acid are obtained from Aldrich (Chicago, Ill.).

In one aspect, a phosphoantigen according to the present invention comprises a compound of Formula (I):

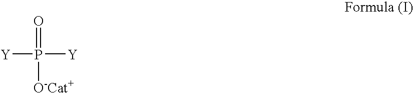

wherein Cat+ represents one (or several, identical or different) organic or mineral cation(s) (including proton);

Y represents O⁻Cat⁺, a C₁-C₆, or more preferably C₁-C₃, alkyl group, a group -A-R, a group -A-PO(O⁻Cat⁺)-Y, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;

A represents O, NH, CHF, CF₂ or CH₂ or R₁—C—R₂ wherein R₁ and R₂ are selected from the group consisting of R, a hydrogen, an hydroxyl (—OH), and an halogen, but may vary independently of each other, respectively; and,

R is a linear, branched, or cyclic, aromatic or not, saturated or unsaturated, C₁-C₂₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof.

Preferably, R₁ and R₂ are defined as any of the R₁ and R₂ shown in Table 1.

In another aspect, a phosphoantigen according to the present invention comprises a compound of Formula (II):

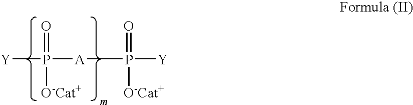

wherein Cat⁺ represents one (or several, identical or different) organic or mineral cation(s) (including proton);

m is an integer from 1 to 3;

Y represents O⁻Cat⁺, a C₁-C₆, or more preferably C₁-C₃, alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;

R and A have the aforementioned meaning.

The term “independently” is used in the present specification to indicate that the variable which is independently applied varies independently from application to application. Thus, in a compound such as R—C—R, wherein “R varies independently as carbon or nitrogen”, both R can be carbon, both R can be nitrogen, or one R can be carbon and the other R nitrogen.

In further embodiments it will be appreciated that the present invention and particularly method for making crystalline phases is suitable for use with structurally related compounds to the ones mentioned specifically herein. In preferred embodiments, the invention also encompasses nucleotides and nucleotide analogs or derivatives or nucleotide-like compounds as well as a bisphosphonate compounds. In a preferred aspect, a compound of Formula II is a bisphosphonate compound, preferably a compound of Formula IIa. A bisphosphonate compound preferably comprises a structure of the Formula (IIa):

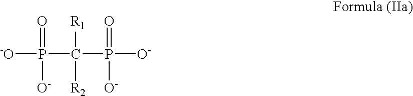

Preferably, R₁ and R₂ are defined as any of the R₁ and R₂ shown in Table 1.

TABLE 1

| Agent | R₁ side chain | R₂ side chain |
|---|---|---|
| Etidronate | —OH | —CH₃ |
| Clodronate | —Cl | —Cl |
| Tiludronate | —H | 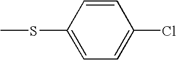 |
| Pamidronate | —OH | —CH₂—CH₂—NH₂ |
| Neridronate | —OH | —(CH₂)₅—NH₂ |
| Olpadronate | —OH | —(CH₂)₂—N(CH₃)₂ |
| Alendronate | —OH | —(CH₂)₃—NH₂ |
| Ibandronate | —OH | 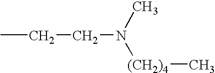 |
| Risedronate | —OH | 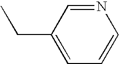 |
| Zoledronate | —OH | 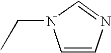 |

Preferably, a compound of the bisphosphonate type is selected from the group consisting of the following compounds or a pharmaceutically acceptable salt thereof, or any hydrate thereof: 3-amino-1-hydroxypropane-1,1-diphosphonic acid (pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-ethidene-bisphosphonic acid, e.g. etidronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-pentylamino)-1-hydroxypropane-1,1-diphosphonic acid, e.g. methyl-pentyl-APD (=BM 21.0955); 1-hydroxy-2-(imidazol-lyl)ethane-1,1-diphosphonic acid; 1-hydroxy-2-(3-pyridinyl)ethane-1,1-diphosphonic acid(risedronic acid), e.g. risedronate, including N-methyl pyridinium salts thereof, for example N-methyl pyridinium iodides such as NE-10244 or NE-10446, 1-(4-chlorophenylthio)methane-1,1-diphosphonic acid(tiludronic acid), e.g. tiludronate; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); 1-hydroxy-2-(imidazo[1,2-a]pyridin-3-yl)ethane-1,1-diphosphonic acid, e.g. YM 529; and 1,1-dichloromethane-1,1-diphosphonic acid (clodronic acid), e.g. clodronate. Preferably the bisphosphonate are compounds which lead to activation of γδ T cells. Examples of commercialized bisphosphonates are shown in Table 1 above, including the identity of R₁ and R₂ for each molecule.

Further examples of preferred phosphoantigens for use according to the present invention comprise the compounds of Formula (III):

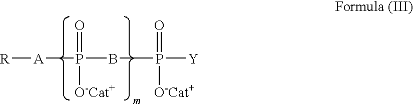

wherein:
Cat⁺ represents one (or several, identical or different) organic or mineral cation(s) (including proton);
m is an integer from 1 to 3;
B is O, NH, or any group capable to be hydrolyzed;
Y represents O⁻Cat⁺, a C₁-C₃ alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
A is O, NH, CHF, CF₂ or CH₂; and,
R is a linear, branched, or cyclic, aromatic or not, saturated or unsaturated, C₁-C₂₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (—CHO), an halogen, an halogenoalkyl, a thiol (—SH), a thioalkyl, a sulfone, a sulfoxide, and a combination thereof Most preferably, said phosphoantigen compound are γδ T cell activators.

In a particular embodiment of any of the formulas disclosed for use in accordance with the invention, the substituents as defined above are substituted by at least one of the substituents as specified above.

Preferably, the substituents are selected from the group consisting of: an (C₁-C₆)alkyl, an (C₂-C₆)alkylenyl, an (C₂-C₆)alkynyl, an (C₂-C₆)epoxyalkyl, an aryl, an heterocycle, an (C₁-C₆)alkoxy, an (C₂-C₆)acyl, an (C₁-C₆)alcohol, a carboxylic group (—COOH), an (C₂-C₆)ester, an (C₁-C₆)amine, an amino group (—NH₂), an amide (—CONH₂), an (C₁-C₆)imine, a nitrile, an hydroxyl (—OH), an aldehyde group (—CHO), an halogen, an (C₁-C₆)halogenoalkyl, a thiol (—SH), a (C₁-C₆)thioalkyl, a (C₁-C₆)sulfone, a (C₁-C₆)sulfoxide, and a combination thereof.

More preferably, the substituents are selected from the group consisting of: an (C₁-C₆)alkyl, an (C₂-C₆)epoxyalkyl, an (C₂-C₆)alkylenyl, an (C₁-C₆)alkoxy, an (C₂-C₆)acyl, an (C₁-C₆)alcohol, an (C₂-C₆)ester, an (C₁-C₆)amine, an (C₁-C₆)imine, an hydroxyl, a aldehyde group, an halogen, an (C₁-C₆)halogenoalkyl and a combination thereof.

Still more preferably, the substituents are selected from the group consisting of: an (C₃-C₆)epoxyalkyl, an (C₁-C₃)alkoxy, an (C₂-C₃)acyl, an (C₁-C₃)alcohol, an (C₂-C₃)ester, an (C₁-C₃)amine, an (C₁-C₃)imine, an hydroxyl, an halogen, an (C₁-C₃)hatogenoalkyl, and a combination thereof Preferably, R is a (C₃-C₂₅)hydrocarbon group, more preferably a (C₅-C₁₀)hydrocarbon group.

In the context of the present invention, the term “alkyl” more specifically means a group such as methyl, ethyl, propyl, isopropyl, butyl, isobutyl, tert-butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, eicosyl, heneicosyl, docosyl and the other isomeric forms thereof. (C₁-C₆)alkyl more specifically means methyl, ethyl, propyl, isopropyl, butyl, isobutyl, tert-butyl, pentyl, hexyl and the other isomeric forms thereof (C₁-C₃)alkyl more specifically means methyl, ethyl, propyl, or isopropyl.

The term “alkenyl” refers to an alkyl group defined hereinabove having at least one unsaturated ethylene bond and the term “alkynyl” refers to an alkyl group defined hereinabove having at least one unsaturated acetylene bond. (C₂-C₆)alkylene includes a ethenyl, a propenyl (1-propenyl or 2-propenyl), a 1- or 2-methylpropenyl, a butenyl (1-butenyl, 2-butenyl, or 3-butenyl), a methylbutenyl, a 2-ethylpropenyl, a pentenyl (1-pentenyl, 2-pentenyl, 3-pentenyl, 4-pentenyl), an hexenyl (1-hexenyl, 2-hexenyl, 3-hexenyl, 4-hexenyl, 5-hexenyl), and the other isomeric forms thereof (C₂-C₆)alkynyl includes ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl and the other isomeric forms thereof.

The term “epoxyalkyl” refers to an alkyl group defined hereinabove having an epoxide group. More particularly, (C₂-C₆)epoxyalkyl includes epoxyethyl, epoxypropyl, epoxybutyl, epoxypentyl, epoxyhexyl and the other isomeric forms thereof (C₂-C₃)epoxyalkyl includes epoxyethyl and epoxypropyl.

The “aryl” groups are mono-, bi- or tri-cyclic aromatic hydrocarbons having from 6 to 18 carbon atoms. Examples include a phenyl, α-naphthyl, β-naphthyl or anthracenyl group, in particular.

“Heterocycle” groups are groups containing 5 to 8 rings comprising one or more heteroatoms, preferably 1 to 5 endocyclic heteroatoms. They may be mono-, bi- or tri-cyclic. They may be aromatic or not. Preferably, and more specifically for R₅, they are aromatic heterocycles. Examples of aromatic heterocycles include pyridine, pyridazine, pyrimidine, pyrazine, furan, thiophene, pyrrole, oxazole, thiazole, isothiazole, imidazole, pyrazole, oxadiazole, triazole, thiadiazole and triazine groups. Examples of bicycles include in particular quinoline, isoquinoline and quinazoline groups (for two 6-membered rings) and indole, benzimidazole, benzoxazole, benzothiazole and indazole (for a 6-membered ring and a 5-membered ring). Nonaromatic heterocycles comprise in particular piperazine, piperidine, etc.

“Alkoxy” groups correspond to the alkyl groups defined hereinabove bonded to the molecule by an —O— (ether) bond. (C₁-C₆)alkoxy includes methoxy, ethoxy, propyloxy, butyloxy, pentyloxy, hexyloxy and the other isomeric forms thereof. (C₁-C₃)alkoxy includes methoxy, ethoxy, propyloxy, and isopropyloxy.

“Alcyl” groups correspond to the alkyl groups defined hereinabove bonded to the molecule by an —CO— (carbonyl) group. (C₂-C₆)acyl includes acetyl, propylacyl, butylacyl, pentylacyl, hexylacyl and the other isomeric forms thereof (C₂-C₃)acyl includes acetyl, propylacyl and isopropylacyl.

“Alcohol” groups correspond to the alkyl groups defined hereinabove containing at least one hydroxyl group. Alcohol can be primary, secondary or tertiary. (C₁-C₆)alcohol includes methanol, ethanol, propanol, butanol, pentanol, hexanol and the other isomeric forms thereof (C₁-C₃)alcohol includes methanol, ethanol, propanol and isopropanol.

“Ester” groups correspond to the alkyl groups defined hereinabove bonded to the molecule by an —COO— (ester) bond. (C₂-C₆)ester includes methylester, ethylester, propylester, butylester, pentylester and the other isomeric forms thereof (C₂-C₃)ester includes methylester and ethylester.

“Amine” groups correspond to the alkyl groups defined hereinabove bonded to the molecule by an —N— (amine) bond. (C₁-C₆)amine includes methylamine, ethylamine, propylamine, butylamine, pentylamine, hexylamine and the other isomeric forms thereof (C₁-C₃)amine includes methylamine, ethylamine, and propylamine.

“Imine” groups correspond to the alkyl groups defined hereinabove having a (—C═N—) bond. (C₁-C₆)imine includes methylimine, ethylimine, propylimine, butylimine, pentylimine, hexylimine and the other isomeric forms thereof (C₁-C₃)imine includes methylimine, ethylimine, and propylimine.

The halogen can be Cl, Br, I, or F, more preferably Br or F.

“Halogenoalkyl” groups correspond to the alkyl groups defined hereinabove having at least one halogen. The groups can be monohalogenated or polyhalogenated containing the same or different halogen atoms. For example, the group can be a trifluoroalkyl (CF₃—R). (C₁-C₆)halogenoalkyl includes halogenomethyl, halogenoethyl, halogenopropyl, halogenobutyl, halogenopentyl, halogenohexyl and the other isomeric forms thereof. (C₁-C₃)hatogenoalkyl includes halogenomethyl, halogenoethyl, and halogenopropyl.

“Thioalkyl” groups correspond to the alkyl groups defined hereinabove bonded to the molecule by an —S— (thioether) bond. (C₁-C₆)thioalkyl includes thiomethyl, thioethyl, thiopropyl, thiobutyl, thiopentyl, thiohexyl and the other isomeric forms thereof. (C₁-C₃)thioalkyl includes thiomethyl, thioethyl, and thiopropyl.

“Sulfone” groups correspond to the alkyl groups defined hereinabove bonded to the molecule by an —SOO— (sulfone) bond. (C₁-C₆)sulfone includes methylsulfone, ethylsulfone, propylsulfone, butylsulfone, pentylsulfone, hexylsulfone and the other isomeric forms thereof (C₁-C₃)sulfone includes methylsulfone, ethylsulfone and propylsulfone.

“Sulfoxyde” groups correspond to the alkyl groups defined hereinabove bonded to the molecule by an —SO— (sulfoxide) group. (C₁-C₆)sulfoxide includes methylsulfoxide, ethylsulfoxide, propylsulfoxide, butylsulfoxide, pentylsulfoxide, hexylsulfoxide and the other isomeric forms thereof (C₁-C₃)sulfoxide includes methylsulfoxide, ethylsulfoxide, propylsulfoxide and isopropylsulfoxide.

“Heteroatom” denotes N, S, or O.

“Nucleoside” refers to a compound composed of any pentose or modified pentose moiety attached to a specific position of a heterocycle or to the natural positions of a purine (9-position) or pyrimidine (1-position) or to the equivalent position in an analog. The term nucleoside includes but is not limited to adenosine, thymine, uridine, cytidine and guanosine.

In a particular embodiment, the hydrocarbon group is a cycloalkylenyl such as a cyclopentadiene or a phenyl, or an heterocycle such as a furan, a pyrrole, a thiophene, a thiazole, an imidazole, a triazole, a pyridine, a pyrimidine, a pyrane, or a pyrazine. Preferably, the cycloalkylenyl or the heterocycle is selected from the group consisting of a cyclopentadiene, a pyrrole or an imidazole. In a preferred embodiment, the cycloalkylenyl or the heterocycle is substituted by an alcohol. Preferably, said alcohol is a (C₁-C₃)alcohol.

In another embodiment, the hydrocarbon group is an alkylenyl with one or several double bonds. Preferably, the alkylenyl group has one double bond. Preferably, the alkylenyl group is a (C₃-C₁₀)alkylenyl group, more preferably a (C₄-C₇)alkylenyl group. Preferably, said alkylenyl group is substituted by at least one functional group. More preferably, the functional group is selected from the group consisting of an hydroxy, an (C₁-C₃)alkoxy, an aldehyde, an (C₂-C₃)acyl, or an (C₂-C₃)ester. In a more preferred embodiment, the hydrocarbon group is butenyl substituted by a group —CH₂OH. Optionally, said alkenyl group can be the isoform trans (E) or cis (Z), more preferably a trans isoform (E). In a most preferred embodiment, the alkylenyl group is the (E)-4-hydroxy-3-methyl-2-butenyl. In an other preferred embodiment, the alkylenyl group is an isopentenyl, a dimethylallyl or an hydroxydimethylallyl.

In an additional embodiment, the hydrocarbon group is an alkyl group substituted by an acyl. More preferably, the hydrocarbon group is an (C₄-C₇)alkyl group substituted by an (C₁-C3)acyl.

In a further preferred embodiment, the phosphoantigen is of formula (IIIa):

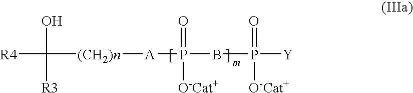

in which:
R₄ is an halogenated (C₁-C₃)alkyl, a (C₁-C₃)alkoxy-(C₁-C₃)alkyl, an halogenated (C₂-C₃)acyl or a (C₁-C₃)alkoxy-(C₂-C₃)acyl,
R₃ is (C₁-C₃)alkyl group,
m is an integer from 1 to 3,
n is an integer from 2 to 20,
B represents O, NH, or any group capable to be hydrolyzed
A represents O, NH, CHF, CF₂ or CH₂,
Y represents O⁻Cat⁺, a C₁-C₃ alkyl group, a group -A-R, or a radical selected from the group consisting of a nucleoside, an oligonucleotide, a nucleic acid, an amino acid, a peptide, a protein, a monosaccharide, an oligosaccharide, a polysaccharide, a fatty acid, a simple lipid, a complex lipid, a folic acid, a tetrahydrofolic acid, a phosphoric acid, an inositol, a vitamin, a co-enzyme, a flavonoid, an aldehyde, an epoxyde and a halohydrin;
R is a linear, branched, or cyclic, aromatic or not, saturated or unsaturated, C₁-C₅₀ hydrocarbon group, optionally interrupted by at least one heteroatom, wherein said hydrocarbon group comprises an alkyl, an alkylenyl, or an alkynyl, preferably an alkyl or an alkylene, which can be substituted by one or several substituents selected from the group consisting of: an alkyl, an alkylenyl, an alkynyl, an epoxyalkyl, an aryl, an heterocycle, an alkoxy, an acyl, an alcohol, a carboxylic group (—COOH), an ester, an amine, an amino group (—NH₂), an amide (—CONH₂), an imine, a nitrile, an hydroxyl (—OH), a aldehyde group (-CHO), an halogen, an halogenoalkyl, a thiol (—SH), a thioalkyl, a sulfone, a sulfoxide, and a combination thereof Most preferably, said phosphoantigen compound are γδ T cell activators, and
Cat⁺ represents one (or several, identical or different) organic or mineral cation(s) (including the proton).

Preferably, R₄ is an halogenated methyl (—CH₂—X, X being an halogen), an halogenated (C₂-C₃)acetyl, or (C₁-C₃)alkoxy-acetyl. The halogenated methyl or acetyl can be mono-, di-, or tri-halogenated. More preferably, R₄ is a CH₂—X group, X represents a halogen atom. Preferably, X is selected from I, Br and Cl. Preferably, R₃ is a methyl or ethyl group. More preferably, R₃ is a methyl. Preferably, B is O and A is O or CH₂. Preferably, n is an integer from 2 to 10, or from 2 to 5. In a more preferred embodiment, n is 2. Preferably, m is 1 or 2. More preferably, m is 1. Preferably, Y is O⁻Cat⁺, or a nucleoside. More preferably, Y is O⁻Cat⁺.

In a most preferred embodiment, n is 2, R₃ is a methyl and R₄ is a halogenated methyl, more preferably a monohalogenated methyl, still more preferably a bromide methyl. In a particularly preferred embodiment, n is 2, R₃ is a methyl, R₄ is a methyl bromide. In a most preferred embodiment, R is 3-(bromomethyl)-3-butanol-1-yl.

In another embodiment, R₄ is a CH₂—X group and A and B represent O.

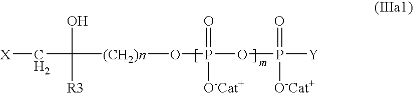

wherein R₃, X, n, m, Y and Cat⁺ have the aforementioned meaning. Preferably, R₃ is a methyl. Preferably, n is 2. Preferably, X is a bromide.

In another embodiment R₄ is a CH₂—X group and B represents O and A represents CH₂.

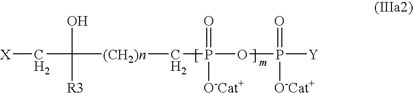

wherein R₃, X, n, m, Y and Cat⁺ have the aforementioned meaning. Preferably, R₃ is a methyl. Preferably, n is 2. Preferably, X is a bromide.

In one preferred embodiment, a phosphoantigen comprises a compound of Formula (IIIa3):

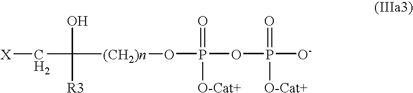

in which X is an halogen (preferably selected from I, Br and Cl), R₃ is a methyl or ethyl group, Cat⁻ represents one (or several, identical or different) organic or mineral cation(s) (including the proton), and n is an integer from 2 to 20. Preferably, R₃ is a methyl. Preferably, n is 2. Preferably, X is a bromide.

In a most preferred embodiment, a phosphoantigen comprises a compound of Formula (A):

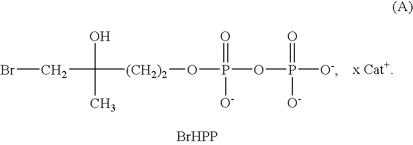

### Preferably x Cat⁺ is 1 or 2 Na⁺.

In another most preferred embodiment, a phosphoantigen comprises a compound of Formula (B):

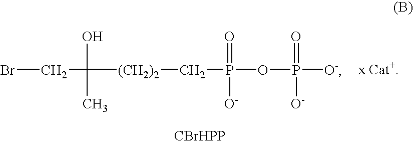

### Preferably x Cat⁺ is 1 or 2 Na⁺.

In a further preferred embodiment, the phosphoantigen is of formula (IIIb):

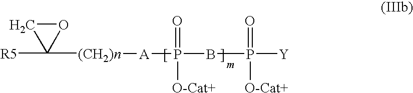

wherein:
n is an integer from 2 to 20,
m is an integer from 1 to 3,
R₅ is a methyl or ethyl group,
B represents O, NH, or any group capabl to be hydrolzed,
A represents O, NH, CHF, CF₂ or CH₂,
Y is O⁻Cat⁺, a nucleoside, or a radical -A-R, wherein R has the aforementioned meaning, and
Cat⁺ represents one (or several, identical or different) organic or mineral cation(s) (including the proton).

Preferably, n is an integer from 2 to 10, or from 2 to 5. In a more preferred embodiment, n is 2. Preferably, R₅ is a methyl. Preferably, Y is O⁻Cat⁺, or a nucleoside. More preferably, Y is O⁻Cat+. Preferably, A is O, NH or CH₂. More preferably, B is O. Preferably, B is O. Preferably, m is 1 or 2. More preferably, m is 1.

For example, a phosphoantigen may comprise a compound of Formula (IIIb1) or (IIIb2):

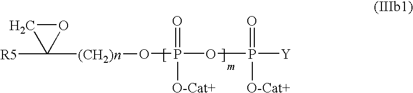

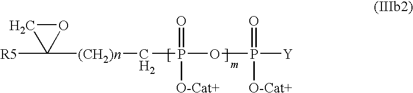

wherein R₅, n, m, Y and Cat⁺ have the above mentioned meaning.

In another preferred embodiment, a phosphoantigen comprises a compound of Formula (IIIb3):

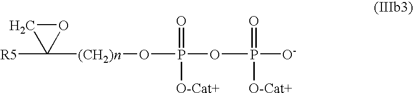

in which R₅ is a methyl or ethyl group, Cat⁺ represents one (or several, identical or different) organic or mineral cation(s) (including the proton), and n is an integer from 2 to 20. Preferably, R₅ is a methyl. Preferably, n is 2.

In another preferred embodiment, a phosphoantigen comprises a compound of Formula (C):

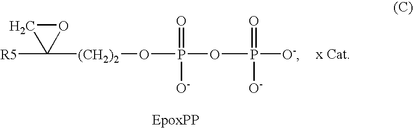

### Preferably x Cat+ is 1 or 2 Na³⁰ .

In a further preferred embodiment, the phosphoantigen is of formula (IIIc):

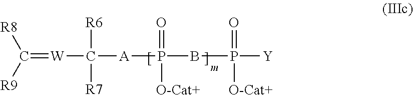

wherein:
R₆, R₇, and R₈ represent, independently from each other, a hydrogen atom or a (C₁-C₃)alkyl group,
R₉ is an (C₂-C₃)acyl, an aldehyde, an (C₁-C₃)alcohol, or an (C₂-C₃)ester,
W is —CH—, —N— or —C—R₁₀,
A is O, NH, CHF, CF₂ or CH₂,
B represents O, NH, or any group capable to be hydrolyzed,
m is an integer from 1 to 3,
Y is O⁻Cat⁺, a nucleoside, or a radical -A-R, wherein R has the aforementioned meaning.
Cat⁻ represents one (or several, identical or different) organic or mineral cation(s) (including the proton).

More preferably, R₆ and R₈ are a methyl and R₇ is hydrogen. More preferably, R₉ is —CH₂—OH, —CHO, —CO—CH₃ or —CO—OCH₃. Optionally, the double-bond between W and C is in conformation trans (E) or cis (Z). More preferably, the double-bond between W and C is in conformation trans (E).

The group Y can permit the design of a prodrug. Therefore, Y is enzymolabile group which can be cleaved in particular regions of the subject. The group Y can also be targeting group. In a preferred embodiment, Y is O⁻Cat⁺, a group —B—R, or a radical selected from the group consisting of a nucleoside, a monosaccharide, an epoxyde and a halohydrin. Preferably, Y is an enzymolabile group. Preferably, Y is O⁻Cat⁺, a group —B—R, or a nucleoside. In a first preferred embodiment, Y is O⁻Cat⁺. In a second preferred embodiment, Y is a nucleoside.

In a preferred embodiment, Cat⁺ is H⁺, Na⁺, NH₄⁺, K⁺, Li⁺, (CH₃CH₂)₃NH⁺.

In a preferred embodiment, B is O or NH. More preferably, B is O.

In a preferred embodiment, A is O, NH or CH₂.

In a preferred embodiment, m is 1 or 2. More preferably, m is 1.

In another example, a phosphoantigen comprises a compound of Formula (IIIc1) or (IIIc2):

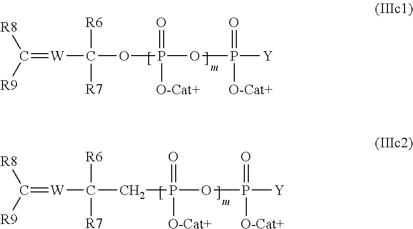

wherein R₆, R₇, R₈, R₉, R₁₀, W, m, Y and Cat⁺ have the above mentioned meaning.

In a preferred embodiment, W is —CH—. Preferably, R₆ and R₇ are hydrogen. Preferably, R₈ is a methyl. Preferably, R₉ is —CH₂—OH.

In another preferred embodiment, a phosphoantigen comprises a compound of Formula (D):

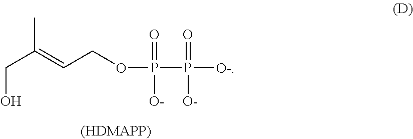

In yet another preferred embodiment, a phosphoantigen comprises a compound of Formula (E):

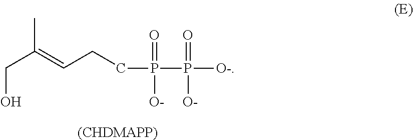

In yet another preferred embodiment, a phosphoantigen comprises a compound of Formula (F):

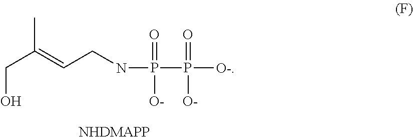

In another example, phosphoantigen comprises a compound of Formula (IIIc3):

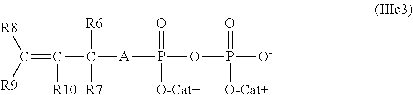

wherein R₆, R₇, R₈, R₉, R₁₀, and A have the above mentioned meaning. Preferably, R_{6,} R₇ and R₉ are hydrogen. Preferably, R₁₀ is a methyl. Preferably, R₈ is —CH₂—OH. Preferably, A is CH₂, NH or O.

In a preferred embodiment, a phosphoantigen comprises a compound of Formula:

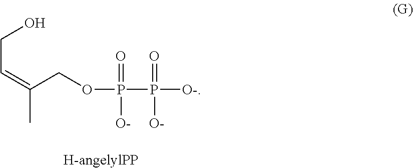

Specific examples of compounds also include: (E)1-pyrophosphonobuta-1,3-diene; (E)1-pyrophosphonopenta-1,3-diene; (E)1-pyrophosphono-4-methylpenta-1,3-diene; (E,E)1-pyrophosphono-4,8-dimethylnona-1,3,7-triene; (E,E,E)1-pyrophosphono-4,8,12-trimethyltrideca-1,3,7,11-tetraene; (E,E)1-triphosphono-4,8-dimethylnona-1,3,7-triene; 4-triphosphono-2-methylbutene; α,β-di-[3-methylpent-3-enyl]-pyrophosphonate; 1-pyrophosphono-3-methylbut-2-ene; α,γ-di-[3-methylbut-2-enyl]-triphosphonate; α,β-di-[3-methylbut-2-enyl]-pyrophosphonate; allyl-pyrophosphonate; allyl-triphosphonate; α,γ-di-allyl-pyrophosphonate; α,β-di-allyl-triphosphonate; (E,E)4-[(5′-pyrophosphono-6′-methyl-penta-2′,4′-dienyloxymethyl)-phenyl]-phenyl-methanone; (E,E)4-[(5′-triphosphono-6′-methyl-penta-2′,4′-dienyloxymethyl)-phenyl]-phenyl-methanone; (E,E,E)[4-(9′-pyrophosphono-2′,6′-dimethyl-nona-2′,6′, 8′-trienyloxymethyl)-phenyl]-phenyl-methanone; (E,E,E)[4-(9′-pyrophosphono-2′,6′,8′-trimethyl-nona-2′,6′,8′-trienyloxymethyl)-phenyl]-phenyl-methanone; 5-pyrophosphono-2-methypentene; 5-triphosphono-2-methypentene; α,γ-di-[4-methylpent-4-enyl]-triphosphonate; 5-pyrophosphono-2-methypent-2-ene; 5-triphosphono-2-methypent-2-ene; 9-pyrophosphono-2,6-dimethynona-2,6-diene; 9-triphosphono-2,6-dimethynona-2,6-diene; α,γ-di-[4,8-dimethylnona-2,6-dienyl]-triphosphonate; 4-pyrophosphono-2-methybutene; 4-methyl-2-oxa-pent-4-enyloxymethylpyrophosphate; 4-methyl-2-oxa-pent-4-enyloxymethyltriphosphate; α,β-di-[4-methyl-2-oxa-pent-4-enyloxymethyl]-pyrophosphate; and α,γ-di-[4-methyl-2-oxa-pent-4-enyloxymethyl]-triphosphate.

In other particular embodiments, the phosphoantigen can be selected from the group consisting of: 3-(halomethyl)-3-butanol-1-yl-diphosphate; 3-(halomethyl)-3-pentanol-1-yl-diphsophate; 4-(halomethyl)-4-pentanol-1-yl-diphosphate; 4-(halomethyl)-4-hexanol-1-yl-diphosphate; 5-(halomethyl)-5-hexanol-1-yl-diphosphate; 5-(halomethyl)-5-heptanol-1-yl-diphosphate; 6-(halomethyl)-6-heptanol-1-yl-diphosphate; 6-(halomethyl)-6-octanol-1-yl-diphosphate; 7-(halomethyl)-7-octanol-1-yl-diphosphate; 7-(halomethyl)-7-nonanol-1-yl-diphosphate; 8-(halomethyl)-8-nonanol-1-yl-diphosphate; 8-(halomethyl)-8-decanol-1-yl-diphosphate; 9-(halomethyl)-9-decanol-1-yl-diphosphate; 9-(halomethyl)-9-undecanol-1-yl-diphosphate; 10-(halomethyl)-10-undecanol-1-yl-diphosphate; 10-(halomethyl)-10-dodecanol-1-yl-diphosphate; 11-(halomethyl)-11-dodecanol-1-yl-diphosphate; 11-(halomethyl)-11-tridecanol-1-yl-diphosphate; 12-(halomethyl)-12-tridecanol-1-yl-diphosphate; 12-(halomethyl)-12-tetradecanol-1-yl-diphosphate; 13-(halomethyl)-13-tetradecanol-1-yl-diphosphate; 13-(halomethyl)-13-pentadecanol-1-yl-diphosphate; 14-(halomethyl)-14-pentadecanol-1-yl-diphosphate; 14-(halomethyl)-14-hexadecanol-1-yl-diphosphate; 15-(halomethyl)-15-hexadecanol-1-yl-diphosphate; 15-(halomethyl)-15-heptadecanol-1-yl-diphosphate; 16-(halomethyl)-16-heptadecanol-1-yl-diphosphate; 16-(halomethyl)-16-octadecanol-1-yl-diphosphate; 17-(halomethyl)-17-octadecanol-1-yl-diphosphate; 17-(halomethyl)-17-nonadecanol-1-yl-diphosphate; 18-(halomethyl)-18-nonadecanol-1-yl-diphosphate; 18-(halomethyl)-18-eicosanol-1-yl-diphosphate; 19-(halomethyl)-19-eicosanol-1-yl-diphosphate; 19-(halomethyl)-19-heneicosanol-1-yl-diphosphate; 20-(halomethyl)-20-heneicosanol-1-yl-diphosphate; 20-(halomethyl)-20-docosanol-1-yl-diphosphate; 21-(halomethyl)-21-docosanol-1-yl-diphosphate; and 21-(halomethyl)-21-tricosanol-1-yl-diphosphate.

More particularly, the phosphoantigen can be selected from the group consisting of: 3-(bromomethyl)-3-butanol-1-yl-diphosphate (BrHPP); 5-bromo-4-hydroxy-4-methylpentyl pyrophosphonate (CBrHPP); 3-(iodomethyl)-3-butanol-1-yl-diphosphate (IHPP); 3-(chloromethyl)-3-butanol-1-yl-diphosphate (ClHPP); 3-(bromomethyl)-3-butanol-1-yl-triphosphate (BrHPPP); 3-(iodomethyl)-3-butanol-1-yl-triphosphate (IHPPP); α,γ-di-[3-(bromomethyl)-3-butanol-1-yl]-triphosphate (diBrHTP); and α,γ-di-[3-(iodomethyl)-3-butanol-1-yl]-triphosphate (dilHTP).

In another particular embodiment, the phosphoantigen can be selected from the group consisting of: 3,4-epoxy-3-methyl-1-butyl-diphosphate (Epox-PP); 3,4,-epoxy-3-methyl-1-butyl-triphosphate (Epox-PPP); α,γ-di-3,4,-epoxy-3-methyl-1-butyl-triphosphate (di-Epox-TP); 3,4-epoxy-3-ethyl-1-butyl-diphosphate; 4,5-epoxy-4-methyl-1-pentyl-diphosphate; 4,5-epoxy-4-ethyl-1-pentyl-diphosphate; 5,6-epoxy-5-methyl-1-hexyl-diphosphate; 5,6-epoxy-5-ethyl-1-hexyl-diphosphate; 6,7-epoxy-6-methyl-1-heptyl-diphosphate; 6,7-epoxy-6-ethyl-1-heptyl-diphosphate; 7,8-epoxy-7-methyl-1-octyl-diphosphate; 7,8-epoxy-7-ethyl-1-octyl-diphosphate; 8,9-epoxy-8-methyl-1-nonyl-diphosphate; 8,9-epoxy-8-ethyl-1-nonyl-diphosphate; 9,10-epoxy-9-methyl-1-decyl-diphosphate; 9,10-epoxy-9-ethyl-1-decyl-diphosphate; 10,11-epoxy-10-methyl-1-undecyl-diphosphate; 10,11-epoxy-10-ethyl-1-undecyl-diphosphate; 11,12-epoxy-11-methyl-1-dodecyl-diphosphate; 11,12-epoxy-11-ethyl-1-dodecyl-diphosphate; 12,13-epoxy-12-methyl-1-tridecyl-diphosphate; 12,13-epoxy-12-ethyl-1-tridecyl-diphosphate; 13,14-epoxy-13-methyl-1-tetradecyl-diphosphate; 13,14-epoxy-13-ethyl-1-tetradecyl-diphosphate; 14,15-epoxy-14-methyl-1-pentadecyl-diphosphate; 14,15-epoxy-14-ethyl-1-pentadecyl-diphosphate; 15,16-epoxy-15-methyl-1-hexadecyl-diphosphate; 15,16-epoxy-15-ethyl-1-hexadecyl-diphosphate; 16,17-epoxy-16-methyl-1-heptadecyl-diphosphate; 16,17-epoxy-16-ethyl-1-heptadecyl-diphosphate; 17,18-epoxy-17-methyl-1-octadecyl-diphosphate; 17,18-epoxy-17-ethyl-1-octadecyl-diphosphate; 18,19-epoxy-18-methyl-1-nonadecyl-diphosphate; 18,19-epoxy-18-ethyl-1-nonadecyl-diphosphate; 19,20-epoxy-19-methyl-1-eicosyl-diphosphate; 19,20-epoxy-19-ethyl-1-eicosyl-diphosphate; 20,21-epoxy-20-methyl-1-heneicosyl-diphosphate; 20,21-epoxy-20-ethyl-1-heneicosyl-diphosphate; 21,22-epoxy-21-methyl-1-docosyl-diphosphate; and 21,22-epoxy-21-ethyl-1-docosyl-diphosphate.

In a further particular embodiment, the phosphoantigen can be selected from the group consisting of: 3,4-epoxy-3-methyl-1-butyl-diphosphate (Epox-PP); 3,4,-epoxy-3-methyl-1-butyl-triphosphate (Epox-PPP); α,γ-di-3,4,-epoxy-3-methyl-1-butyl-triphosphate (di-Epox-TP); and uridine 5′-triphosphate-(3,4-epoxy methyl butyl) (Epox-UTP).

In another preferred embodiment, the phosphoantigen can be selected from the group consisting of: (E)-4-hydroxy-3-methyl-2-butenyl pyrophosphate (HDMAPP) and (E)-5-hydroxy-4-methylpent-3-enyl pyrophosphonate (CHDMAPP).

These compounds may be produced according to various techniques known per se in the art, some of which being disclosed in PCT Publications nos. WO 00/12516, WO 00/12519, WO 03/050128, WO 02/083720 and WO 03/009855, the disclosures of which are incorporated herein by reference.

In one preferred embodiment, the phosphoantigen is a γδ T cell activator and is a compound described in any one of PCT publication nos. WO 00/12516, WO 00/12519, WO 03/050128, WO 02/083720, WO 03/009855 and WO 05/054258, the disclosures of which Formulas and specific structures as well as synthesis methods are incorporated herein by reference. In another preferred embodiment, the phosphoantigen is a γδ T cell activator and is a compound selected from the group consisting of HDMAPP, CHDMAPP, NHDMAPP, H-angelyIPP, Epox-PP, BrHPP and CBrHPP.

Alternatively, although less potent in their functions as a γδ T cell activator, other activators for use in the present invention are phosphoantigens disclosed in WO 95/20673, isopentenyl pyrophosphate (IPP) (U.S. Pat. No. 5,639,653) and 3-methylbut-3-enyl pyrophosphonate (C-IPP). The disclosures of both references are incorporated herein by reference. Also encompassed are compounds that contain a phosphate moiety and act as γδ T cell inhibitors; one example is a compound disclosed in U.S. Pat. No. 6,624,151 B1, the disclosure of which is incorporated herein by reference.

Each of the foregoing references relating to compounds and their synthesis are incorporated herein by reference.

Dosage of Preferred γδ T cell Activators

As discussed, preferred compounds are selected which increase the biological activity of γδ T cells, preferably increasing the activation or proliferation of γδ T cells, particularly increasing cytokine secretion from γδ T cells or increasing the cytolytic activity of γδ T cells, with or without also stimulating the expansion of γδ T cells. For example, a γδ T cell activator allows the cytokine secretion by γδ T cells to be increased at least 2, 3, 4, 10, 50, 100-fold, as determined in vitro. Cytokine secretion and cytolytic activity can be assessed using any appropriate in vitro assay, or those described herein.

Optionally, in another aspect, the present invention relates to methods for the treatment of a disease, especially a proliferative disease, and more preferably a solid tumor, particularly a solid tumor having metastases, where a γδ T cell activator, especially a γδ T cell activator according to formulas I to III. In the framework of the present invention, the expression “Formulas I to III”, designate all compounds derived from Formulas I to III: I, II, IIa, III, IIIa, IIIa1, IIIa2, IIIa3, A, B, IIIb, IIIb1, IIIb2, IIIb3, C, IIIc, IIIc1, IIIc2, IIIc3, D, E, F and G. Preferably, the γδ T cell activator selected from the group consisting of BrHPP, CBrHPP, CHDMAPP, NHMDMAPP, HDMAPP and epoxPP, is administered in an amount and under conditions sufficient to stimulate the expansion of the γδ T cell population in a subject, particularly to reach 30-90% of total circulating lymphocytes, typically 40-90%, more preferably from 50-90%, conjointly with a chemotherapeutic agent. In typical embodiments, the conjoint therapy with a chemotherapeutic agent allows (e.g. does not prevent) the selective expansion of γδ T cells in a subject, to reach more than 10%, 20%, 30%, 40%, 50%, or up to 60-90% of total circulating lymphocytes. Percentage of total circulating lymphocytes can be determined according to methods known in the art. A preferred method for determining the percentage of γδ T cells in total circulating lymphocytes is by flow cytometry, examples of appropriate protocols described in the examples herein.

In another embodiment, the present invention relates to methods for the treatment of a disease, especially a proliferative disease, and more preferably a solid tumor, particularly a solid tumor, where a γδ T cell activator, especially a γδ T cell activator according to formulas I to III, especially γδ T cell activator selected from the group consisting of BrHPP, CBrHPP, CHDMAPP, NHMDMAPP, HDMAPP and epoxPP, is administered in an amount and under conditions sufficient to stimulate the expansion of the γδ T cell population in a subject, particularly to increase by more than 2-fold the number of γδ T cells in a subject, typically at least 4, 5 or 10-fold, more preferably at least 20-fold, conjointly with a chemotherapeutic agent. In typical embodiments, the conjoint administration with a chemotherapeutic agent allows the selective expansion of γδ T cells in a subject, to increase by at least 2, 4, 5, 10, 20, or 50-fold the number of γδ T cells in a subject, more preferably at least 100 fold. The number of γδ T cells in a subject is preferably assessed by obtaining a blood sample from a patient before and after administration of said γδ T cell activator and determining the difference in number of γδ T cells present in the sample. A preferred method for determining the number of γδ T cells by flow cytometry, examples of appropriate protocols described in the examples herein.

In another aspect, the present invention relates to methods for the treatment of a disease, especially a proliferative disease, and more preferably a solid tumor, particularly a solid tumor having metastases, where a γδ T cell activator, especially a γδ T cell activator according to formulas I to III, especially γδ T cell activator selected from the group consisting of BrHPP, CBrHPP, CHDMAPP, NHMDMAPP, HDMAPP and epoxPP, is administered in an amount and under conditions sufficient to stimulate the expansion of the γδ T cell population in a subject, particularly to reach a circulating γδ T cell count of at least 500 γδ T cells/mm³ in a subject, typically at least 1000 γδ T cells/mm³, more preferably at least 2000 γδ T cells/mm³, conjointly with a chemotherapeutic agent. The circulating γδ T cell count in a subject is preferably assessed by obtaining a blood sample from a patient before and after administration of said γδ T cell activator and determining the number of γδ T cells in a given volume of sample. A preferred method for determining the number of γδ T cells by flow cytometry, examples of appropriate protocols described in the examples herein.

In a further aspect, the present invention relates to an in vivo regimen for the treatment of a proliferative disease, especially a solid tumor and more particularly a solid tumor having metastases, where a γδ T cell activator, especially a γδ T cell activator according to formulas I to III, especially γδ T cell activator selected from the group consisting of BrHPP, CBrHPP, CHDMAPP, NHMDMAPP, HDMAPP and epoxPP, is administered to a warm-blooded animal, especially a human, in a dose that is higher (preferably at least 10%, 20%, 30% higher) than the single administration Efficient Concentration value giving half of the maximum effect (EC50) of γδ T cell biological activity or population expansion, or more preferably a dose that is at least 50%, or more preferably at least 60%, 75%, 85% or preferably between about 50% and 100% of the single administration Efficient Concentration value giving the maximum effect, conjointly with a chemotherapeutic agent.

Preferably, dosage (single administration) of a γδ T cell activator compound of formula I to III for treatment is between about 1 μg/kg and about 1.2 g/kg. It will be appreciated that the above dosages related to a group of compounds, and that each particular compound may vary in optimal doses, as further described herein for exemplary compounds. Nevertheless, compounds are preferably administered in a dose sufficient to significantly increase the biological activity of γδ T cells or to significantly increase the γδ T cell population in a subject. Said dose is preferably administered to the human by intravenous (i.v.) administration during 2 to 180 min, preferably 2 to 120 min, more preferably during about 5 to about 60 min, or most preferably during about 30 min or during about 60 min. In preferred exemplary compounds, a compound of formula II to III is administered in a dosage (single administration) between about 0. 1 mg/kg and about 1.2 g/kg, preferably between about 10 mg/kg and about 1.2 g/kg, more preferably between about 5 mg/kg and about 100 mg/kg, even more preferably between about 5 ag/kg and 60 mg/kg. In the framework of the present invention, the expression “Formulas II to III”, designate all compounds derived from Formulas II to III: II, IIa, III, IIIa, IIIa1, IIIa2, IIIa3, A, B, IIIb, IIIb1, IIIb2, IIIb3, C, IIIc, IIIc1, IIIc2, IIIc3, D, E, F and G. Most preferably, dosage (single administration) for three-weekly or four-weekly treatment (treatment every three weeks or every third week) is between about 0. 1 mg/kg and about 1.2 g/kg, preferably between about 10 mg/kg and about 1.2 g/kg, more preferably between about 5 mg/kg and about 100 mg/kg, even more preferably between about 5 μg/kg and 60 mg/kg. In preferred exemplary compounds, a compound of formula IIIc, especially, a compound of formula D, E, F or G, is administered in a dosage (single administration) between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg. Most preferably, dosage (single administration) for three-weekly or four-weekly treatment (treatment every three weeks or every third week) is between about 1 μg/kg and about 100 mg/kg, preferably between about 10 μg/kg and about 20 mg/kg, more preferably between about 20 μg/kg and about 5 mg/kg, even more preferably between about 20 μg/kg and 2.5 mg/kg. Further detail on dosages and administration and examples of dose response experiments using γδ T cell activator in mice and primate models are provided in co-pending PCT Application no. PCT/FR03/03560 filed 2 Dec. 2003, the disclosure of which is incorporated herein by reference.

Unless otherwise indicated, the dosages for administration to a warm blooded animal, particularly humans provided herein are indicated in pure form (anionic form) of the respective compound. Purity level for the active ingredient depending on the synthesis batch can be used to adjust the dosage from actual to anionic form and vice-versa.

### DETAILED DESCRIPTION OF PREFERRED TREATMENT REGIMENS

### Renal Cell Carconima (RCC)

In specific embodiments, patients with renal cell carcinoma are administered a prophylactically or therapeutically effective amount of a γδ T cell activator in combination with the administration of a prophylactically or therapeutically effective amount of one or more other therapies useful for renal cell carcinoma treatment or management including but not limited to: an anti-angiogenic therapy or an HDAC inhibitor, most preferably a receptor tyrosine kinase inhibitor, an inhibitor or ras or raf kinase, or an antibody which specifically binds an angiogenic factor (e.g. VEGF). Most preferably the receptor tyrosine kinase inhibitor is an agent selected from the group consisting of: SU011248, PTK787 and BAY 43-9006 (sorafenib). The most common subtype of RCC, accounting for 60-70% of cases, comprises clear or conventional cell carcinoma and is characterized by frequent inactivation of the Von Hippel-Lindau (VHL) tumor suppressor gene. RCC is generally considered resistant to conventional cytotoxic drugs.

### Breast Cancer

In specific embodiments, patients with breast cancer are administered a prophylactically or therapeutically effective amount of a γδ T cell activator in combination with a prophylactically or therapeutically effective amount of one or more other therapies useful for breast cancer treatment or management including, but not limited to: doxorubicin, epirubicin, the combination of doxorubicin and cyclophosphamide (AC), the combination of cyclophosphamide, doxorubicin and 5-fluorouracil (CAF), the combination of cyclophosphamide, epirubicin and 5-fluorouracil (CEF), tamoxifen, or the combination of tamoxifen and cytotoxic chemotherapy, an anti-angiogenic therapy or an HDAC inhibitor alone or in combination with any of the preceding. In certain embodiments, patients with metastatic breast cancer are administered a prophylactically or therapeutically effective amount of a γδ T cell activator in combination with the administration of a prophylactically or therapeutically effective amount of taxanes such as docetaxel and paclitaxel. In other embodiments, a patients with node-positive, localized breast cancer are administered a prophylactically or therapeutically effective amount of a γδ T cell activator in combination with the administration of a prophylactically or therapeutically effective amount of taxanes plus standard doxorubicin and cyclophosphamide for adjuvant treatment of node-positive, localized breast cancer.

### Treatment of Colon Cancer

In specific embodiments, patients with colon cancer are administered a prophylactically or therapeutically effective amount of a yo T cell activator in combination with the administration of a prophylactically or therapeutically effective amount of one or more other therapies useful for colon cancer treatment or management including but not limited to: the combination of 5-FU and leucovorin, the combination of 5-FU and levamisole, irinotecan (CPT-11) or the combination of irinotecan, 5-FU and leucovorin (IFL), oxaliplatin, or an anti-angiogenic therapy or an HDAC inhibitor alone or in combination with any of the preceding.

### Treatment of Prostate Cancer

In specific embodiments, patients with prostate cancer are administered a prophylactically or therapeutically effective amount of a γδ T cell activator in combination with the administration of a prophylactically or therapeutically effective amount of one or more other therapies useful for prostate cancer treatment or management including but not limited to: extenal-beam radiation therapy, interstitial implantation of radioisotopes (i.e., palladium, and Iridium), chemotherapy regimens reported to produce subjective improvement in symptoms and reduction in PSA level including docetaxel, paclitaxel, estramustine/docetaxel, estramustine/etoposide, estramustine/vinblastine, and estramustine/paclitaxel, or an anti-angiogenic therapy or an HDAC inhibitor, alone or in combination with any of the preceding.

### Treatment of Melanoma

In specific embodiments, patients with melanoma are administered a prophylactically or therapeutically effective amount of a γδ T cell activator in combination with the administration of a prophylactically or therapeutically effective amount of one or more other therapies useful for melanoma cancer treatment or management including but not limited to: dacarbazine (DTIC), nitrosoureas such as carmustine (BCNU) and lomustine (CCNU), agents with modest single agent activity including vinca alkaloids, platinum compounds, and taxanes, the Dartmouth regimen (cisplatin, BCNU, and DTIC), or an anti-angiogenic therapy or an HDAC inhibitor, alone or in combination with any of the preceding.

### Treatment of Ovarian Cancer

In specific embodiments, patients with ovarian cancer are administered a prophylactically or therapeutically effective amount of a γδ T cell activator in combination with a prophylactically or therapeutically effective amount of one or more other therapies useful for ovarian cancer treatment or management including, but not limited to: intraperitoneal radiation therapy, total abdominal and pelvic radiation therapy, cisplatin, oxaliplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-fluorouracil (5-FU) and leucovorin, etoposide, liposomal doxorubicin, gerucitabine or topotecan, or an anti-angiogenic therapy or an HDAC inhibitor alone or in combination with any of the preceding. In a particular embodiment, patients with ovarian cancer that is platinum-refractory are administered a prophylactically or therapeutically effective amount of a 7 T cell activator in combination with the administration of a prophylactically or therapeutically effective amount of Taxol. The invention encompasses the treatment of patients with refractory ovarian cancer including administration of ifosfamide in patients with disease that is platinum-refractory, hexamethylmelamine (HAM) as salvage chemotherapy after failure of cisplatin-based combination regimens, and tamoxifen in patients with detectable levels of cytoplasmic estrogen receptor on their tumors.

### Treatment of Lung Cancers

In specific embodiments, patients with non-small cell lung cancer and small lung cell cancer are administered a prophylactically or therapeutically effective amount of a γδ T cell activator in combination with the administration of a prophylactically or therapeutically effective amount of one or more other therapies useful for lung cancer treatment or management including but not limited to: thoracic radiation therapy, cisplatin, vincristine, doxorubicin, and etoposide, alone or in combination, the combination of cyclophosphamide, doxorubicin, vincristine/etoposide, and cisplatin (CAV/EP), or an anti-angiogenic therapy or an HDAC inhibitor alone or in combination with any of the preceding.

In other specific embodiments, patients with non-small lung cell cancer are administered a prophylactically or therapeutically effective amount of a γδ T cell activator in combination with the administration of a prophylactically or therapeutically effective amount of one or more other therapies useful for lung cancer treatment or management including but not limited to: palliative radiation therapy, the combination of cisplatin, vinblastine and mitomycin, the combination of cisplatin and vinorelbine, paclitaxel, docetaxel or gemcitabine, the combination of carboplatin and paclitaxel, or an anti-angiogenic therapy or an HDAC inhibitor alone or in combination with any of the preceding.

### Treatment of Chronic Myelogenous Leukaemia (CML)

In specific embodiments, patients with CML are administered a prophylactically or therapeutically effective amount of a γδ T cell activator in combination with the administration of a prophylactically or therapeutically effective amount of one or more other therapies useful for CML treatment or management including but not limited to: compositions that inhibit the tyrosine kinases ABL, ARG, PDGFRalpha, PDGFRbeta, and/or c-KIT, or imitanib mesylate (STI571, Glivec™, Gleevec™, Novartis).

### Treatment Regimens

Treatment with a chemotherapeutic agent (e.g. any one or two of an anti-angiogenic therapy (e.g. tyrosine kinase inhibitor), paclitaxel, carboplatin, gemcitabine, cisplatin, vinorelbine, HDAC inhibitor, radiation) and a γδ T cell activator can be carried out according to any suitable administration regimen. The γδ T cell activator may be administered through any of several different routes, typically by injection or oral administration. Injection may be carried out into various tissues, such as by intravenous, intra-peritoneal, intra-arterial, intra-muscular, intra-dermic, subcutaneous, etc. Particularly preferred is intravenous injection. The γδ T cell activator can be administered before, at the same time or after the chemotherapeutic agent is administered. Generally, the γδ T cell activator will be administered no more than several (4, 5, 6, or 7) days before or after treatment with the chemotherapeutic agent. Most preferably, however, the γδ T cell activator is administered at substantially the same time as the chemotherapeutic agent is administered, preferably within (e.g. before or after) 1 day, or within 1 week, 2 weeks, 3 weeks or 4 weeks, 48 hours, 24 hours or more preferably within 12 or within 6 hours of treatment with the chemotherapeutic agent.

In one preferred aspect, the γδ T cell activator is administered conjointly with a chemotherapeutic agent, where the chemotherapeutic agent is dosed daily, or 2, 3, 4, 5 or 6 days per week. In a preferred embodiment, the chemotherapeutic agent is an anti-angiogenic therapy. The γδ T cell activator is administered at substantially the same time as the chemotherapeutic agent is administered, preferably within 48 hours, 24 hours or more preferably within 12 or within 6 hours of the chemotherapeutic agent.

In one aspect, the γδ T cell activator is administered once during the course or preferably once during a cycle of chemotherapeutic agent therapy.

In another aspect the γδ T cell activator is administered two or more times during the course or preferably once during a cycle of chemotherapeutic agent therapy. The course of a preferred cycle for administering the γδ T cell activator is on an at least 1-weekly cycle, but more preferably at least a 2-weekly cycle (at least about 14 days), or more preferably at least 3-weekly or 4-weekly, though cycles anywhere between 2-weekly and 8-weekly are preferred, for example 5-weekly, 6-weekly, 7-weekly or 8-weekly. In a preferred embodiment, the γδ T cell activator is administered only the first day of said cycle.

The present invention relates especially to the treatment of a disease, especially a tumor, characterized in that a chemotherapeutic compound is administered more than once, with a weekly or three-weekly, interval to a human in a dose that is calculated according to the formula (T)

single dose (mg/kg)=(x to y)×N (T)

where N (a whole or fractional number) is the number of weeks between treatments of chemotherapeutic agent (about one to about eight weeks), that is N is about 1 to about 8, preferably about 1, 2 or 3;

more preferably, the treatment dose is calculated according to the formula U

(single dose (mg/kg)=(x to y)×N)×L; (U)

where N (a whole or fractional number) is the number of weeks between treatments (about one to about eight weeks), that is N is about 1 to about 8, preferably about 1, 2 or 3, where L (a whole or fractional number) is the number of treatments administered, that L is 1, 2, 3, 5, 6 or greater;

more preferably, the treatment dose is calculated according to the formula V

(((single dose (mg/kg)=(x to y)×N)×L)+P; (V)

where N (a whole or fractional number) is the number of weeks between treatments (about one to about eight weeks), that is N is about 1 to about 8, preferably about 1, 2 or 3, where L (a whole or fractional number) is the number of treatments administered, that L is 1, 2, 3, 5, 6 or greater, and where P (a whole or fractional number) is the number of weeks between the treatment last dose of the treatment and the first dose of the following treatment (e.g. the “lag” or “break”) and that P is about 1, 2, 3 or greater;

the administration of the compound of Formulas I to III taking place:
(a) once,
   (1) within 1 to 7 days of a chemotherapeutic agent dose, optionally wherein said dose is the first dose of the chemotherapy treatment cycle;
   (2) within about 1 week, 2 weeks, 3 weeks or 4 weeks of the last chemotherapeutic agent dose of the chemotherapy treatment cycle, for example during the lag time (P) prior to the first chemotherapeutic agent dose in a second chemotherapy cycle; or
(b) in at least 2, 3, 4 or more doses, and about three-weekly, four-weekly to about eight weekly, preferably about five-weekly, six-weekly, seven-weekly or eight-weekly, the dose of the compound of Formulas IIIa to IIIb, especially compounds of formula A, B or C being administered preferably within 1 to 7 days, or within 2 weeks, 3 weeks or 4 weeks of a chemotherapeutic agent dose; and
   at a human in a dose of:
(1) for compositions of Formula IIIa to IIIb:
   a. between about 0.1 mg/kg and 100 mg/kg, or preferably between about 10 mg/kg and about 100 mg/kg, preferably between about 5 mg/kg and about 60 mg/kg, or about 10, 15, 20, 30, 40 or 50 mg/kg; or
b. between about 5 mg and 10 g, or preferably between about 200 mg and about 10 g, preferably between about 200 g and about 1.2 g, or between about 200 mg/m² and 400, 600, 800, 1000, 1200, 1400, 1600 or 1800 mg/m² of body surface area; and
(2) for compositions of Formulas IIIc:
   a. between about 1 μg/kg and about 100 mg/kg, or preferably between about 10 μg/kg and about 20 mg/kg, preferably between about 20 μg/kg and about 5 mg/kg, between about 20 μg/kg and 2.5 mg/kg, or about 0.1, 0.2, 0.3, 0.4 or 0.5 mg/kg; or
   b. between about 50 μg and 10 g, or preferably between about 100 μg and about 2 g, preferably between about 100 μg and about 0.5 g, or between about 5 mg/m² and 10, 50, 100, 200, 300, 500 or 1000 mg/m² of body surface area.
      The administration of γδ T cell activator preferably takes place by i.v. infusion.

### Weekly Administration of Chemotherapeutic

The present regimen will generally be used for chemotherapeutic drugs such as taxanes, platinum drugs, anti-metabolites, alkylating agents, for example paclitaxel, carboplatin, 5-FU, cisplatin, vinorelbin, gemcitabine, and certain anti-angiogenic therapies (monoclonal antibodies such as Avastin (Genentech Inc.), IMC-1121B (Imclone Systems Inc., CDP-791 (Cellteeh Inc.)), etc., where the chemotherapeutic agent is administered less than once per week.

In one exemplary embodiment, the chemotherapeutic agent is administered on a 1-weekly cycle for 3 weeks or 6 weeks, or longer, and the T cell activator occurs once or multiple times. When the γδ T cell activator is administered only once during a chemotherapeutic treatment, it is preferably administered at the start, during a break, or at the end of the treatment, preferably within about 1 day, 1 week, 2 weeks or 3 weeks of the last dose of the chemotherapeutic agent (e.g. at the end of a particular course of therapy, or during a break or gap in a chemotherapy treatment). In another aspect, when the administration of the γδ T cell activator occurs in multiple doses, it is preferably administered on the first day of a 2-weekly to 8-weekly cycle. In a preferred embodiment, the chemotherapeutic agent is administered on a 1-weekly cycle for 3 weeks and the γδ T cell activator is administered only the first day of this cycle. Thus, a 3-weekly cycle is used for the γδ T cell activator and a 1-weekly cycle can be used for the chemotherapeutic agent, for example over a course of three or six weeks. The three or six week long therapy regimen can be repeated as many times as necessary, with or without a break (lag) in between. Exemplary administration schemes are set forth as follows, any or which can be repeated one or more times:
Day 1: chemotherapeutic agent(s) and γδ T cell activator
Day 8: chemotherapeutic agent(s)
Day 15: chemotherapeutic agent(s)
   In another example the following scheme is used:
Day 1: chemotherapeutic agent(s)
Day 8: chemotherapeutic agent(s)
Day 15: chemotherapeutic agent(s)
Day 22, 29 or 36: γδ T cell activator
   In another example the following scheme is used:
Day 1: chemotherapeutic agent(s) and optionally and γδ T cell activator
Day 8: chemotherapeutic agent(s)
Day 15: chemotherapeutic agent(s)
Day 22: chemotherapeutic agent(s)
Day 29: chemotherapeutic agent(s)
Day 36: chemotherapeutic agent(s)
Day 43, 50 or 57: γδ T cell activator

### 3-Weekly Administration of Chemotherapeutic

In other exemplary administration regimens, particularly in maintenance therapy or classical regimens with certain chemotherapeutics, a 3-weekly cycle is used for the chemotherapeutic agent. The γδ T cell activator can be administered once or multiple times. In one example, both the γδ T cell activator and are administered in 3-weekly cycles, and optionally are administered on the same day. In another example, the γδ T cell activator is administered after the last dose in a chemotherapy cycle, particularly during a break (lag) between cycles or treatment.

The present regimen will generally be used for chemotherapeutic drugs such as taxanes, platinum drugs, anti-metabolites, alkylating agents, for example paclitaxel, carboplatin, 5-FU, ciplatin, vinorelbin, gemcitabine, etc. as well as certain anti-angiogenic therapies that are dosed less than daily (e.g. monoclonal antibodies such as Avastin (Genentech Inc.), IMC-1121B (Imclone Systems Inc., CDP-791 (Celltech Inc.,)).

In one exemplary embodiment, the chemotherapeutic agent is administered on a 3-weekly cycle for 3 weeks, 6 weeks, 9 weeks, or 12 weeks, or longer, and the γδ T cell activator occurs on the first day of a 2-weekly to 8-weekly cycle. In a preferred embodiment, the chemotherapeutic agent is administered on a 3-weekly cycle for at least 3 weeks and the γδ T cell activator is administered within 1 day, 1 week, 2 weeks, 3 weeks or 4 weeks of the chemotherapeutic agent.

It will be appreciated that the foregoing 3-, 6- or other week cycle of therapy can be repeated as many times as needed, with or without a break in treatment between successive 3 week cycles. Moreover, the γδ T cell activator need not be administered in every 3 week chemotherapy cycle. At the end of each cycle of chemotherapy, the cycle of dosing may be repeated for as long as clinically tolerated and the tumor is under control or until tumor regression. In exemplary chemotherapeutic agent regimens, administrations of chemotherapeutic agent take place 1-weekly for 6 weeks, followed by an interval (for example 6 weeks), followed by administrations of chemotherapeutic agent 3-weekly for a desired duration, such as at least 6 months or 12 months for maintenance therapy. As mentioned, a subject can receive one, or will preferably be treated for at least two cycles of γδ T cell activator, or more preferably for at least three cycles.

For example, for the treatment of lung cancer (NSCLC), standard paclitaxel regimen is the chemotherapeutic agent(s) and is administered every 3 weeks (dosage 175 mg/m² or 225 mg/m²). In another embodiment, a standard paclitaxel/carboplatin regimen is the chemotherapeutic agent(s), and paclitaxel is administered at a dose of 100 mg/m² at days 1, 8, 15, and carboplatin at a dose of AUC 6.0 at day 1, in a 4 week cycle. In another regimen, paclitaxel (200 mg/m² at day 1) plus gemcitabine (1000 mg/m² at days 1 and 8), with both regimens repeated at 3-week intervals, is used. In yet another regiment, docetaxel is administered once-every-3-weeks or docetaxel 36 mg/m² weekly for 6 consecutive weeks, followed by 2 weeks of break. In yet another regimen, docetaxel/cisplatin are used conjointly. In yet another regimen, gemcitabine is used with cisplatin, the regimen should be administered on a 21-day or 28-day, schedule, with cisplatin administered on day 1 and gemcitabine on days 1 and 8. (gemcitabine usually at 1000 mg/m²).

Daily (or More than Once-Weekly) Dosing of Chemotherapeutic

Administration regimens having daily or multiple doses per week of chemotherapeutic agents will generally be used for orally available agents, including for example HDAC inhibitors, and certain anti-angiogenic therapies such as receptor tyrosine kinase inhibitors and raf or ras kinase inhibitors. Additionally, chemotherapeutic agents such as taxanes, platinum drugs, anti-metabolites, alkylating agents, for example paclitaxel, carboplatin, 5-FU, cisplatin, vinorelbin, and gemcitabine having significant toxicity at high (e.g. MTD) doses when administered in a weekly regimen can be administered as lower doses on a more frequent basis. The latter lower dose regimen for chemotherapeutic drugs is referred to as chronic low-dose or metronomic therapy. Metronomic therapy with certain cytotoxic drugs may be optimal for enhancing their damaging effects to proliferating tumor vasculature. Metronomic chemotherapy generally refers to a schedule of chemotherapy given at lower doses to allow more frequent administration without the induction of myelosuppression seen with maximum tolerated dose (MTD) regimens. This type of regimen is also called antiangiogenic scheduling due to the fact that slowly proliferating (angiogenic) endothelial cells are more efficiently targeted by metronomic chemotherapy than by MTD regimens, resulting in inhibition of tumor growth due to insufficient neovascularization. γδ T cell activators can be used with metronomic cytotoxic regimens and further combined with VEGF/VEGFR blockade for further enhanced antitumor response.

In one exemplary embodiment, the chemotherapeutic agent is administered daily, or alternatively on 2, 3, 4, 5, or 6 days per week, for 1 week, 3 weeks or 6 weeks, or longer (e.g. until disease progression), and the γδ T cell activator occurs once or multiple times. When the γδ T cell activator is administered only once during a chemotherapeutic treatment, it is preferably administered at the start, during a break, or the end of the treatment, preferably within about 1 day, 1 week, 2 weeks or 3 weeks of the last dose of the chemotherapeutic agent (e.g. at the end of a particular course of therapy, or during a break or gap in a chemotherapy treatment).

In another aspect, when the administration of the γδ T cell activator occurs in multiple doses, it is preferably administered on the first day of a 2-weekly to 8-weekly cycle, or a 2-weekly to 4-weekly cycle (that is, an about 14 to 28 day weeks repeating cycle). In a preferred embodiment, the chemotherapeutic agent is administered daily and the γδ T cell activator is administered on a 2 to 8 week cycle.

In one example, a daily regimen is used for treatment with orally available anti-angiogenic therapies such as receptor tyrosine kinase inhibitors, ras kinase inhibitors and raf kinase inhibitors. In a preferred regimen, SU011248 is be administered (orally) once daily (about 50 mg) for four weeks followed by a two-week rest period. In another example, PTK787 (Novartis Pharmaceuticals, Hanover, N.J.) is administered daily at a dose of at least about 1,500 mg. In another example, BAY 43-9006 (sorafenib, Bayer, Germany) is administered daily at a dose of at least about 400 mg bid. The regimens can be used for the treatment of a wide range of solid tumors. In a second example of a regimen for conjoint treatment with receptor tyrosine kinase inhibitors, the receptor tyrosine kinase inhibitor (e.g. SU011248) is administered daily for a treatment period (e.g. four weeks) and the γδ T cell activator is administered during the rest period following the treatment period, preferably within 1 week or within 2 weeks of the end of the treatment period.

In another example, a daily regimen is used for HDAC inhibitors in solid and hematological tumors, preferably lymphocytic leukaemia and androgen independent prostate cancer, as well as peripheral T-cell lymphoma and cutaneous T-cell lymphoma. HDAC inhibitors and conjoint phosphoantigen treatment according to the invention can be expected to be useful in the treatment particularly of lymphomas, myelocytic leukemia, breast cancer, lung cancer, hepatocellular cancer, malignant melanoma and gastrointestinal cancer (Boyle et al. Pigment Cell Res. June 2005;18(3):160-6; and Wiedmann and Caca, Curr Cancer Drug Targets. May 2005;5(3):171-93.)). HDAC inhibitors trigger both mitochondria-mediated apoptosis and caspase-independent autophagic cell death, indicating potential benefit of HDAC inhibitors in treating cancers with apoptotic defects.

In a preferred regimen, suberoylanilide hydroxamic acid (SAHA) can be administered (orally) once daily (400 mg qd), twice daily (200 mg bid), and a twice daily for 3 consecutive days every week (300 mg bid). The regimen can be used for the treatment of a wide range of solid tumors.

In one exemplary regimen conjoint therapy is used for the treatment of colorectal cancer, γδ T cells having been reported to have activity against colon carcinoma cells (Corvaisier et al. J Immunol. (2005) 175(8):5481-8). A γδ T cell activator is administered in a dose as provided herein either once or in repeated doses separated by at least 2, 3, 4, 6 or 8 weeks, and SAHA is administered to a individual having colorectal cancer (orally) once daily (400 mg qd), twice daily (200 mg bid), and a twice daily for 3 consecutive days every week (300 mg bid). Preferably the treatment further comprises conjoint therapy with 5-FU which has been demonstrated to be synergistic with HDAC inhibitors in colorectal cancer.

### Conjoint Administration of Cytokine

In other embodiments, the methods of the invention comprise further administering a cytokine. While the compounds of the invention may be used with or without further administration, in a preferred aspect a cytokine can be administered, wherein said cytokine is capable of increasing the expansion of a γδ T cell population treated with a γδ T cell activator compound, preferably wherein the cytokine is capable of inducing an expansion of a γδ T cell population which is greater than the expansion resulting from administration of the γδ T cell activator compound in the absence of said cytokine. A preferred cytokine is an interleukin-2 polypeptide.

A cytokine having γδ T cell proliferation inducing activity, most preferably the interleukin-2 polypeptide, is administered at low doses, typically over a period of time comprised between 1 and 10 days. The γδ T cell activator is preferably administered in a single dose, and typically at the beginning of a cycle.

In preferred aspects, a cytokine, most preferably IL-2, is administered daily for up to about 10 days, preferably for a period of between about 3 and 10 days, or most preferably for about 7 days. Preferably, the administration of the cytokine begins on the same day (e.g. within 24 hours of) as administration of the γδ T cell activator. It will be appreciated that the cytokine can be administered in any suitable scheme within said regimen of between about 3 and 10 days. For example, in one aspect the cytokine is administered each day, while in other aspects the cytokine need not be administered on each day.

The following examples will serve to further illustrate the present invention without, at the same time, however, constituting any limitation thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### Examples

### Example 1

### Flow Cytometry Detection of T Cell Amplification

Blood samples (4 ml) were withdrawn into EDTA containing tubes. Tubes were shipped overnight at room temperature (RT) before flow cytometry analyses.

Peripheral lymphocytes were analyzed by flow cytometry on total blood, after triple staining with anti-Vdelta2FITC, anti-CD3PE and anti-CD25PC5 antibodies (Vdelta2-FITC : IMMU389 clone, Immunotech-Beckman-Coulter, Marseilles, France; CD3-PE : UCHTI clone, Immunotech-Beckman-Coulter; CD25PC5 : M-A251 clone, Becton Dickinson, Le Pont de Claix, France).

Briefly, 100 μl patient blood was incubated 15 min at RT with 10 μl anti-Vdelta2-FITC, 10 μl anti-CD3-PE and 10 μl anti-CD25PC5 antibodies. Antibodies were washed with 3 ml 1× PBS, centrifuged for 4 min at 1300 rpm at RT and supernatant was discarded. Red cells were lysed with the OptiLyse B reagent (Immunotech-Beckman-Coulter, Marseilles, France) according to the manufacturer's instructions. At the final step, stained white blood cells were recovered by centrifugation and resuspended in 300 μl 1× PBS+0.2% PFA. Flow cytometry analysis on viable cells was performed on a FacsSCAN apparatus (Becton Dickinson) with the FacsDiva software.

### Example 2

### Phosphostim-Induced Amplification of γδ T Cells in Chemotherapy-Treated Patients

PHOSPHOST™ (Bromohydrin pyrophosphate, BrHPP) was evaluated in an escalating dose clinical trial in patients with advanced/metastatic solid tumors. The therapeutic regimen and pharmaco-toxicology of BrHPP in combination with low dose of IL-2 have previously been characterized in non human primates. The patients received at least 3 injections of BrHPP 3 weeks apart, concomitant with low dose of IL-2 (1 million IU/m²/day). Pharmacological response was monitored all along the treatment. Based on the results of the phase I trial, a multicenter phase II study on metastatic RCC patients has been scheduled. The clinical drug product was a 200 mg/vial of lyophilized Phosphostim (expressed in mg equivalent of BrHPP anionic form.). The formulation of Phosphostim was reconstituted immediately prior to use with 2 ml of water for injections to make a 100 mg/ml solution. The reconstituted product was diluted in a total volume of 100 ml of ringer lactate buffer infusion vehicle. Phosphostim was administered intravenously over 1 hour.

The clinical trial was therefore a phase I, single arm, open-label, national, multi-center, dose-escalation trial in sequential cohorts of patients with advanced/metastatic solid tumors.

All patient cohorts received repeated 3-week cycles of Phosphostim alone for the first cycle and Phosphostim in combination with IL-2 for the subsequent cycles.
first cycle: Phosphostim alone,
subsequent cycles: Phosphostim in combination with IL-2

Patients were sequentially allotted to cohorts of progressively higher dose levels of Phosphostim. Cohorts of 3-6 patients were used to identify the MTD. The definition of MTD was based on the tolerability observed in the first and second cycles of treatment. Each patient received Phosphostim in combination with IL-2 until disease progression, patient refusal or unacceptable toxicity occurred. The objective tumor response was assessed after 3 cycles (i.e., every 9 weeks).

Cohorts of 3-6 patients were sequentially enrolled to progressively higher dose levels of Phosphostim. The initial dose of Phosphostim was 200 mg/m². Dose level 1 is provided for dose modification in case individual patients experience DLT at dose level 1 during therapy.

The IL-2 was administered subcutaneously, daily over 7 days starting on the day of Phosphostim infusion (10 min after starting Phosphostim perfusion when IL-2 is administered concomitantly).

### Pharmacodynamic Assessment

Drug plasma levels were measured during the first and the second cycles in all patients. Whole blood for full PD analyses was taken on day 1 and at several time points between day 1 and day 21 for cycles 1 and 2. Six (6) ml of blood were removed pre-dose and at Day 6, Day 8, and Day 12. During the cycles 3, 4, 5 and 6, additional PD analyses were performed. Six (6) ml of blood were removed pre-dose and at Day 6 at each mentioned cycles. The volume of blood withdrawn for pharmacodynamic determinations from each patient did not exceed 24 ml for cycles 1 and 2 and 12 ml for each subsequent cycle. Biological effect was monitored throughout the trial and studied on PBMC and plasma. The biological effect study included:

Whole blood sample for amplification follow up of γδ T cells by immunomonitoring.

Cytolytic activity of the PBMC by in vitro cytotoxic assay (optional).

### Means of Immuno-Monitoring

Immunomonitoring of the different T cell subpopulations was performed by flow cytometry by the Immunology Lab.

These flow cytometry experiments were completed by Blood Cell Count (BCC) and classical hematology performed under standard procedure.

Collection of Blood

The samples will consist of 6 ml (3 ml×2 tubes) whole blood, drawn in vacutainers with anticoagulant agent at the designated times.

### Results

Patients treated with chemotherapeutic regimens within 6 months, 3 months or 1 month preceding treatment with Phosphostim were analyzed for their ability to mount a gamma delta T cell expansion. While several patients received multiple chemotherapeutic regimens prior to treatment with Phosphostim, only the chemotherapy regimens immediately prior to (within the months preceding) or with Phosphostim therapy are listed below. Results demonstrated that chemotherapy treatment, including alkylating agents such as metal salts, antimetabolites such as pyrimidine analogues, plant derivatives such as topoisomerase inhibitors, and specifically anti-angiogenic agents can be used in combination with gamma delta T cell activation without preventing gamma delta T cell amplification in vivo.

Patient 1 (0035) was treated for colon cancer and had received a course of Oxaliplatin/5FU/5FU Bolus/Elvorine (100 mg/m²-400 mg/m²-2400 mg/m²-200 mg/m²) followed by a course of Cetuximab/Cetuximab/Irinotecan (250 mg/m²-400 mg/m²-350 mg/m²). The patient was treated with two cycles of Phosphostim (1500 mg/m2), the first cycle of Phosphostim administered within six months of the end of Cetuximab/Cetuximab/Irinotecan therapy. Pharmacodynamic assessment following the second administration of Phosphostim (i.e. Phosphostim in combination with IL-2) demonstrated a significant (11× amplification rate) amplification of Vγ9Vδ2 T cells as determined by flow cytometry.

Patient 2 (0039) was treated for colon cancer and had received prior therapies Oxaliplatin/5FU/5FU Bolus/Elvorine (100 mg/m²-2400 mg/m²-400 mg/m²-200mg/m² ) followed by Erbitux/Irinotecan therapy ending within three months preceding the first cycle of Phosphostim. The patient was treated with two cycles of Phosphostim (1500 mg/ml). Pharmacodynamic assessment following the second administration of Phosphostim (i.e. Phosphostim in combination with IL-2) demonstrated a significant (6.4× amplification rate) amplification of Vγ9Vδ2 T cells as determined by flow cytometry.

Patient 3 (0018) was treated for metastatic renal cell carcinoma and had received prior therapies tyrosine kinase inhibitor (BAY 43-9006 (Nexavar® (sorafenib tosylate)) and anti-angiogenic therapy Avastin, the latter cycle of therapy ending within a month preceding the first cycle of Phosphostim. The patient was treated with two cycles of Phosphostim (1800 mg/m²). Pharmacodynamic assessment following the second administration of Phosphostim (i.e. Phosphostim in combination with IL-2) demonstrated a significant (6.2× amplification rate) amplification of Vγ9Vδ2 T cells as determined by flow cytometry, and a 72 weeks PFS (progression free survival).

Patient 4 (0017) was treated for colon cancer and had received prior therapies Oxaliplatin/5FU/5FU Bolus/Elvorine/Vinflumine (85 mg/m²-500 mg/m²-400 mg/m²-100 g/m²-240 mg/m² ) ending within three months preceding the first cycle of Phosphostim. The patient was treated with three cycles of Phosphostim (1200 mg/m²). His PFS was 39 weeks. Pharmacodynamic assessment following the second administration of Phosphostim (i.e. Phosphostim in combination with IL-2) demonstrated an amplification of Vγ9Vδ2 T cells (amplification rate of 4.3×).

### Example 3

### Phosphostim-Induced Amplification of γδ T Cells from Patients Undergoing Gleevec Therapy

The study intends to test the sensitivity of γ962 T cells from oncology patients to BrHPP. It aims at identifying cancers and or situations in which a BrHPP treatment would be relevant and those for which it would be less or not beneficial. In brief, a small sample of blood is sufficient to prepare PBMCs and culture in the presence of BrHPP. A result of the level of in vitro amplification of gamma-delta cells by BrHPP can be obtained in about 8 days.

Blood was collected from 19 patients having CML (chronic myelogenous leukemia) and undergoing treatment with imitanib mesylate (STI571, Glivec™, Gleevec™, Novartis). Upon reception of blood, 20 ml blood samples were treated by Ficoll gradient to isolate peripheral lymphocytes, which were then frozen. The lymphocytes were cultured in a 96-well plate (1 million cells/ml) for 8 days in the presence of BrHPP. The percentage of γ962 T cells were then determined by a phenotypic assay (flow cytometry).

In such small scale culture assay, a patient is considered to be responsive to an agonist following 8 days of in vitro stimulation according to two criteria: the percentage of γ962 T cells in culture as well as the rate of amplification of these cells. The amplification rate corresponds to the ratio of number of total γ962 T cells at the end of culture to the total γ962 T cells at the start of culture. The qualitative evaluation of these two criteria was divided into four levels of response, according to the Table 2.

TABLE 2

| | Criteria | | | |
|---|---|---|---|---|
| | % of Vdelta2⁺ cells | | Absolute Vdelta2⁺ cell | |
| Level | | Result | amplification | |
| Definition | Limits | value | Limits | Result value |
| Level 1 | Less than 10% | 0 | Less than x2 | 0 |
| Level 2 | From 10 to 20% | + | From x2 to x8 | + |
| Level 3 | From 20 to 40% | ++ | From x8 to x32 | ++ |
| Level 4 | More than 40% | +++ | More than x32 | +++ |

A patient is considered sensitive to BrHPP if the sum of the values of the two criteria is ++ or +++. A classification of 0 or + is considered non-sensitive.

Of the 19 patients evaluated for sensitivity to BrHPP-induced amplification of γ962 T cells, 18 patients had values of either ++ or +++ and therefore responsive to BrHPP induced amplification. One patient was non-sensitive. γ962 T cells from CML patients treated with Gleevec therefore remain sensitive to γ962T cell agonists such as BrHPP.

### Example 4

### BRHPP-Induced Amplification of γδ T Cells in Monkeys Previously Treated with Imatinib Mesylate (Gleevec™)

The objective of this study is to assess the toxicological potential of a repeated sequential treatment performed in a similar or more stringent therapeutic regimen expected for human treatment in combination with imatinib mesylate (GLEEVEC™ Novartis—standard treatment for Chronic Myeloid Leukemia). The purpose of this study is mainly focused on pharmacological impact of imatinib mesylate on γ982 T cells expansion.

The design is based on two groups: one group of 6 animals treated with BrHPP in combination with imatinib mesylate, a control group (6 animals) with imatinib mesylate alone as a reference and an absolute control group. The toxicity of the therapeutic regimen of our experimental drug BrHPP alone has been documented in the first study performed in the same species, at the same dose level (50 & 180 mg/kg) in the same regimen.

Animals are treated, starting from day 0, and on a daily basis until day 29, with imatinib mesylate (oral—15 mg/kg of GLEEVEC 100 mg. On days 1 and 15 animals are treated with BrHPP (iv, intravenous, 50 or 180 mg/kg), and on day 1 through day 5 and day 15 through 19 animals are also treated with IL-2/adesleukin (PROLEUKIN Chiron, bolus subcutaneous daily injection). Dosage of BrHPP was performed 4 hours after oral dosing of imatinib on the corresponding days. The treatment period is ended on day 29. Day 29 corresponds to 14 days following the last 60-minute infusion of BrHPP. At 14 days after the last BrHPP infusion it is expected that the numbers of γ982 T cells will have generally gone back to baseline. γ982 T cell proliferation was assessed at days 6 and 20.

The results of this study are summarized in FIG. 2. FIG. 2 shows assessments at day 6 and 20, and presents γ982 T cell amplification expressed as the percentage of γ982 T cells among total cells. γδT cell proliferation was not significantly different when BrHPP is administered alone or in combination with Gleevec, confirming that Gleevec does not significantly impair the proliferation of phosphoantigen stimulated γδT cells.

### Example 5

### Study of the Combination of BrHPP and Several Tyrosine Kinase Inhibitors (TKI) on Amplification of Vd2/Vg9 T Cells in NOD SCID Mice

The aim of this study is to assess that the combination of BrHPP and several tyrosine kinase inhibitors (Gleevec, Sorafenib and Sutent) does not modify the amplificative properties of Phosphostim on human γ9δ2 T cells in NOD SCID mice.

### Materials and Methods

### Cells and Culture Condition

Cell line: human PBMC whose γ9δ2 T cells percentage is higher than 0.5% and which has been previously tested for the “sensitivity test”. They were thawed one hour before injection and maintained for one hour in complete medium (RPMI 1640 supplemented with 2 mM L-glutamine, 1 mM Sodium Pyruvate (all from Gibco-BRL, Life Science, Invitrogen) and 10% heat inactivated Fetal Calf Serum (Fetal clone)) in incubator at 37° C.

### Antibodies

FITC anti human V82,Immunotech, ref 1464
PerCpCy5.5 anti human CD3, Pharmingen, ref 552552
APC anti human Vγ9
APC Cy7 anti human CD45, Pharmingen, ref 348815
PE anti human CD56, Pharmingen, ref 556647

### Animals

Mice used included (a) NOD SCID female mice aged 8-10 weeks from Charles River Laboratories. Mice were fed and housed under sterilized conditions in Innate-Pharma's animal facility, and (b)NOD SCID female mice aged 10-12 weeks bred at Innate Pharma.

### Experimental Design.

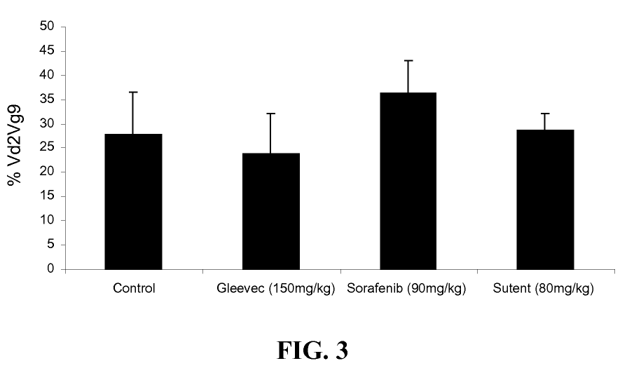

Each group contained 5 mice. Following transfer of human PBMC cells described above, the 4 groups received BrHPP (50 mg/kg, i.p.) on day 0 and IL-2 (2 M/m², s.c.) every day from day 0 to day 4. The treated group received TKI from day 0 for 3 to 5 days (Gleevec was administered at a dose of 150 mg/kg, administered orally, for 4 days, Sorafenib was administered at the dose of 90 mg/kg, os, for 3 days, Sutent was administered at the dose of 80 mg/kg, os, for 3 days). On day 8 mice were sacrificed, cells were collected by peritoneal lavage, and γ9δ2 T cell proliferation was assessed by flow cytometry. FIG. 3 shows γ9δ2 T cell amplification expressed as the percentage of γ9δ2 T cells among total cells.

### Results:

FIG. 3 confirms that the injection of a TKI at the beginning of the treatment does not impair γδT cell proliferation.

1-105. (canceled)

106. A method for treating a proliferative disease in a subject comprising administering to a subject in need thereof a γδ T cell activator conjointly with a chemotherapeutic agent.

107. The method of claim 106, wherein the chemotherapeutic agent is a tyrosine kinase inhibitor:
a) capable of inhibiting bcr/abl;
b) that is a competitive inhibitor at the ATP-binding site of Bcr/Abl;
c) selected from the group consisting of imatinib, SU4312, XL647, XL999, PKC412, AEE788, OSI-930, OSI-817, DMPQ, MLN518, lestaurinib, gefitiib, OSI-774, lapatinib, PD-166326, NSC 680410, tyrphostin AG 957, AP-23464, AP-234604, SKI-606, dasatinib, nilotinib, NS-187, and CGP16030;
d) selected from the group consisting of imatinib, dasatinib, and nilotinib;
c) that does not impair the proliferation of γδ T cells;
f) that inhibits a receptor tyrosine kinase selected from the group consisting of VEGFR1, VEGFR-2.3 PDGFR-alpha, PDGFR -beta,CSF-1,RET, Flt-3, c-Kit, p38 alpha and FGFR-1;
g) that inhibits a receptor tyrosine kinase selected from the group consisting of VEGFR-1, VEGFR-2, VEGFR-3, PDGFR-beta, Flt-3 and c-Kit;
h) selected from the group consisting of sorafenib and sunitinib;
i) capable of inhiibiting a receptor tyrosine kinase selected from the group consisting of VEGFR-1, VEGFR-2, VEGI,R-3, PDGFR-alpha, PDGFR-beta, CSF-1R, Flt-3, RET and c-Kit; or
j) selected from the group consisting of VEGFR-1, PDGFR-alpha, PDGFR, c-Kit and bcr/abl.

108. The method of claim 107, wherein treatment with said tyrosine kinase inhibitor together with a γδ T cell activator maintains the residual disease below the detection limit as established using PCR gene amplification technique.

109. The method of claim 106, wherein said disease is a tumor.

110. The method of claim 109, wherein said tumor is selected from the group consisting of:
a) a CML, ALL or GIST cancer;
b) characterized by a gene or protein mutation selected from the group consisting of c-ABL, BCR-ARL, c-KIT or PDGFR; and
c) characterized by aberrant or increased kiinase activity signaling activity.

111. The method of claim 109, wherein said chemotherapeutic agent is a tyrosine kinase inhibitor:
a) capable of inhibiting a receptor tyrosine kinase selected from the group consisting of VEGFR-1, VEGFR-2, VEGFR-3, PDGFR-alpha, PDGFR-beta, Flt-3, c-Kit, p38 alpha, RET, c-RAF, b-RAF, bcr/abl and FGFR-1;
b) that is capable of inhibiting a receptor tyrosine kinase selected from the group consisting of abl, bcr/abl, c-Kit and PDGFR;
c) that is a competitive inhibitor at the ATP-binding site of Bcr/Abl; or
d) selected from the group consisting of imatinib, PD-166326, NSC 680410, tyrphostin, AP-23464, AP-234604, SKI-606, dasatinib, nilotinib, NS-187, and CGP16030.

112. The method of claim 109 wherein said tumor is characterized by a gene or protein mutation selected from the group consisting of c-ABL, BCR-ABL, c-KIT and PDGFR or by an aberrant or increased tyrosine kinase signaling activity, or by a mutation in a tyrosine kinase.

113. The method of claim 112, wherein the subject is treated for so long as necessary to bring numbers of cell expressing a mutated kinase to a predetermined level, or to undetectable levels.

114. The method of claim 109, wherein the tumor is a lymphoma or leukemia, in particular CML.

115. The method of claim 114, wherein the tyrosine kinase inhibitor is selected from the group consisting of imatinib, PD-166326, NSC 680410, tyrphostin, AP-23464, AP-234604, SKI-606, dasatinib, nilotinib, NS-187, and CGPP16030.

116. The method of claim 106, wherein said method of treatment induces a toxicity not higher than the toxicity of the tyrosine kinase inhibitor alone.

117. The method of claim 107, wherein the γδ T cell activator is administered at least twice, wherein successive administrations of γδ T cell activator are separated by at least 7, 10, 14 or 20 days, and the tyrosine kinase inhibitor is administered daily or weekly during treatment with the period of treatment of the γδ T cell activator.

118. The method of claim 106, wherein treatment with a chemotherapeutic agent conjointly with a γδ T cell activator maintains the residual disease below the detection limit as established using PCR gene amplification technique.

119. The method of claim 106, wherein the γδ T cell activator is administered within 3 months after a treatment with the chemotherapeutic agent.

120. The method of claim 106, wherein said γδ T cell activator and said chemotherapeutic agent are administered in an amount effective to induce proliferation of γδ T cells in said mammal.

121. The method of claim 106, wherein said γδ T cell activator and said chemotherapeutic agent are administered in an amount effective to induce activation of γδ T cells in said mammal.

122. A pharmaceutical composition comprising a γδ T cell activator compound and a tyrosine kinase inhibitor.

123. The pharmaceutical composition of claim 122, wherein said tyrosine kinase inhibitor is imatinib.

124. The pharmaceutical composition of claim 122, wherein said γδ T cell activator is selected from the group consisting of compounds of Formula IIIc.

125. The pharmaceutical composition of claim 124, wherein said tyrosine kinase inhibitor is selected from the group consisting of imatinib, SU4312. XL647, XL999, PKC412, AEE788, OSI-930, OSI-817, DMPQ, MLN518, lestaurnib, gefitinib, OSI-774, lapatinib PD-166326, NSC 680410, tyrphostin AG 957, AP-23464, AP-234604, SKI-606, dasatinib, nilotinib, NS-187, and CGP16030 or is capable of inhibiting a receptor tyrosine kinase selected in the group consisting of VEGFR-1, VEGFR-2, VEGFR-3, PDGFR-alpha, PDGFR-beta, CSF-IR, Flt-3, c-Kit and RET.

126. The pharmaceutical composition of claim 122, wherein said tyrosine kinase inhibitor:
a) is capable of inhibiting a receptor tyrosine kinase selected from the group consisting of VEGFR-1, VEGFR-2, VEGFR-3, PDGFR-alpha, PDGFR-beta, Flt-3, c-Kit, p38 alpha, RET, c-RAF, b-RAF, bcr/abl and FGFR-1;
b) is capable of inhibiting a receptor tyrosine kinase selected from the group consisting of abl, bcr/abl, c-Kit and PDGFR; or
c) is selected from the group consisting of imatinib, SU4312, XL647, XL999, PKC412 AEE788. OSI-930, OSI817, DMPQ, MLN518, lestaurinib, geitiLnib, OS1-774, lapatinib, PD-166326, NSC 680410, tyrphostin AG957, AP-23464, AP-234604, SKI-606, dasatinib, nilotinib, NS-187, and CGP16030.

127. A method of treating a subject comprising:
a) administering to subject having CML, ALL or GIST a tyrosine kinase inhibitor capable of inhibiting a receptor tyrosine kinase selected from the group consisting of bcr/abl c-Kit and PDGFR in combination with a γδ T cell activator;
b) administering to a subject having mRCC, RCC or GIST sLuiitinib in combination with a γδ T cell activator; or
c) administering to a subject having mRCC or RCC sorafenib in combination with a γδ T cell activator.

128. The method of claim 127, wherein said method comprises administering to subject having CML, ALL or GIST a tyrosine kinase inhibitor capable of inhibiting a receptor tyrosine kinase selected from the group consisting of bcr/abl c-Kit and PDGFR in combination with a γδ T cell activator and wherein said tyrosine kinase inhibitor is imatinib.

129. A method for enhancing the killing of a target cell comprising:
a) activating a γδ T cell by bringing said γδ T cell into contact with a chemotherapeutic agent; or
b) activating a γδ T cell in the mammal by bringing said γδ T cell into contact with a γδ T cell activator.
